(19) United States
(12) Patent Application Publication
(10) Pub. No.: US 2014/0200259 A1
(43) Pub. Date: Jul. 17, 2014

(54) RNAi-MEDIATED INHIBITION OF SELECT RECEPTOR TYROSINE KINASES FOR TREATMENT OF PATHOLOGIC OCULAR NEOVASCULARIZATION-RELATED CONDITIONS

(71) Applicant: Novartis AG, Basel (CH)

(72) Inventors: David P. Bingaman, Weatherford, TX (US); Jon E. Chatterton, Aliso Viejo, CA (US)

(73) Assignee: Novartis AG, Basel (CH)

(21) Appl. No.: 14/192,451

(22) Filed: Feb. 27, 2014

Related U.S. Application Data
(63) Continuation of application No. 13/940,503, filed on Jul. 12, 2013.
Application No. 13/940,503 is a division of application No. 13/019,655, filed on Feb. 2, 2011, now abandoned.
Application No. 13/019,655 is a division of application No. 11/678,571, filed on Feb. 23, 2007, now abandoned.
(60) Provisional application No. 60/776,062, filed on Feb. 23, 2006.

(51) Int. Cl. C12N 15/113

(52) U.S. Cl. CPC C12N 15/113
USPC 514/44 A

(57) ABSTRACT

RNA interference is provided for inhibiton of expression of select receptor tyrosine kinase (RTK) targets in ocular neovascularization-related conditions, including those cellular changes resulting from the signal transduction activity of the select RTK targets that lead directly or indirectly to ocular NV, abnormal angiogenesis, retinal vascular permeability, retinal edema, diabetic retinopathy particularly proliferative diabetic retinopathy, diabetic macular edema, exudative age-related macular degeneration, sequela associated with retinal ischemia, and posterior segment neovascularization.

The present application is a continuation of U.S. patent application Ser. No. 13/940,503 filed Jul. 12, 2013, which is a divisional of U.S. patent application Ser. No. 13/019,655 filed Feb. 2, 2011 (now abandoned), which is a divisional of U.S. application Ser. No. 11/678,571 filed Feb. 23, 2007 (now abandoned), which claims priority to U.S. Provisional Application Ser. No. 60/776,062, filed Feb. 23, 2006.

### FIELD OF THE INVENTION

The present invention relates to the field of interfering RNA compositions for inhibition of expression of select receptor tyrosine kinase (RTK) targets in pathologic ocular neovascularization-related conditions.

### BACKGROUND OF THE INVENTION

Pathologic ocular neovascularization (NV) and related conditions occur as a cascade of events that progresses from an initiating stimulus to the formation of abnormal new capillaries. The stimulus appears to be the elaboration of various proangiogenic growth factors such as vascular endothelial growth factor (VEGF), platelet-derived growth factor (PDGF), and angiopoetins, among others. Following initiation of the angiogenic cascade, the capillary basement membrane and extracellular matrix are degraded and capillary endothelial cell proliferation and migration occur. Endothelial sprouts anastomose to form tubes with subsequent patent lumen formation. The new capillaries commonly have increased vascular permeability or leakiness due to immature barrier function, which can lead to tissue edema. Differentiation into a mature capillary is indicated by the presence of a continuous basement membrane and normal endothelial junctions between other endothelial cells and pericytes; however, this differentiation process is often impaired during pathologic conditions.

Retinal NV is observed in retinal ischemia, proliferative and nonproliferative diabetic retinopathy (PDR and NPDR, respectively), retinopathy of prematurity (ROP), central and branch retinal vein occlusion, and age-related macular degeneration (AMD). The retina includes choriocapillaries that form the choroid and are responsible for providing nourishment to the retina, Bruch's membrane that acts as a filter between the retinal pigment epithelium (RPE) and the choriocapillaries, and the RPE that secretes angiogenic and anti-angiogenic factors responsible for, among many other things, the growth and recession of blood vessels.

NV can include damage to Bruch's membrane which then allows growth factor to come in contact with the choriocapillaries and initiating the process of angiogenesis. The new capillaries can break through the RPE as well as Bruch's membrane to form a new vascular layer above the RPE. Leakage of the vascular layer leads to wet or exudative AMD and subsequent loss of cones and rods that are vital to vision.

Exudative AMD and PDR are the major causes of acquired blindness in developed countries and are characterized by pathologic posterior segment neovascularization (PSNV). The PSNV found in exudative AMD is characterized as pathologic choroidal NV, whereas PDR exhibits preretinal NV. In spite of the prevalence of PSNV, treatment strategies are few and palliative at best. Approved treatments for the PSNV in exudative AMD include laser photocoagulation and photodynamic therapy with VISUDYNE®; both therapies involve laser-induced occlusion of affected vasculature and are associated with localized laser-induced damage to the retina. For patients with PDR, grid or panretinal laser photocoagulation and surgical interventions, such as vitrectomy and removal of preretinal membranes, are the only options currently available. Several different compounds are being evaluated clinically for the pharmacologic treatment of PSNV, including RETAANE® (Alcon Research, Ltd.), Lucentis™, Avastin™ (Genentech), adPEDF (GenVec), squalamine (Genaera), CA4P (OxiGENE), VEGF trap (Regeneron), LY333531 (Lilly), and siRNAs targeting VEGF (Cand S, Acuity) and VEGFR-1 (Sirna-027, Sirna Therapeutics). Macugen® (Eyetech/Pfizer), an anti-VEGF aptamer injected intravitreally, has recently been approved for such use. In addition, an “Ang-trap” (Amgen) is in development to sequester the ligand for Tie-2 and an siRNA against RTP801, a downstream target of HIF-1, is under development (Quark Biotech).

Macular edema is the major cause of vision loss in diabetic patients, whereas preretinal neovascularization (PDR) is the major cause of legal blindness. Diabetes mellitus is characterized by persistent hyperglycemia that produces reversible and irreversible pathologic changes within the microvasculature of various organs. Diabetic retinopathy (DR), therefore, is a retinal microvascular disease that is manifested as a cascade of stages with increasing levels of severity and worsening prognoses for vision. Major risk factors reported for developing diabetic retinopathy include the duration of diabetes mellitus, quality of glycemic control, and presence of systemic hypertension. DR is broadly classified into 2 major clinical stages: nonproliferative diabetic retinopathy (NPDR) and proliferative diabetic retinopathy (PDR), where the term “proliferative” refers to the presence of preretinal neovascularization as previously stated.

Nonproliferative diabetic retinopathy (NPDR) and subsequent macular edema are associated, in part, with retinal ischemia that results from the retinal microvasculopathy induced by persistent hyperglycemia. NPDR encompasses a range of clinical subcategories which include initial “background” DR, where small multifocal changes are observed within the retina (e.g., microaneurysms, “dot-blot” hemorrhages, and nerve fiber layer infarcts), through preproliferative DR, which immediately precedes the development of PSNV. The histopathologic hallmarks of NPDR are retinal microaneurysms, capillary basement membrane thickening, endothelial cell and pericyte loss, and eventual capillary occlusion leading to regional ischemia. Data accumulated from animal models and empirical human studies show that retinal ischemia is often associated with increased local levels of proinflammatory and/or proangiogenic growth factors and cytokines, such as prostaglandin E2, vascular endothelial growth factor (VEGF), insulin-like growth factor-1 (IGF-1), Angiopoeitin 2, etc. Diabetic macular edema can be seen during either NPDR or PDR, however, it often is observed in the latter stages of NPDR and is a prognostic indicator of progression towards development of the most severe stage, PDR.

At present, no pharmacologic therapy is approved for the treatment of NPDR and/or macular edema. The current standard of care is laser photocoagulation, which is used to stabilize or resolve macular edema and retard the progression toward PDR. Laser photocoagulation may reduce retinal ischemia by destroying healthy tissue and thereby decreasing metabolic demand; it also may modulate the expression and production of various cytokines and trophic factors. Similar to the exudative AMD treatments, laser photocoagulation in diabetic patients is a cytodestructive procedure and the visual field of the treated eye is irreversibly compromised. Other than diabetic macular edema, retinal edema can be observed in various other posterior segment diseases, such as posterior uveitis, branch retinal vein occlusion, surgically induced inflammation, endophthalmitis (sterile and non-sterile), scleritis, and episcleritis, etc.

Small molecule receptor tyrosine kinase (RTK) inhibitors (RTKi) such as PKC412 (CPG 41251), PTK787, and MAE 87 have been described that act on VEGF receptors and inhibit retinal neovascularization or choroidal neovascularization in mice. Each of these molecules inhibits multiple kinases. For example, PKC412 inhibits KDR (hVEGFR-2), PDGFR-β, Flk-1 (mVEGFR-2), and Flt-1 (VEGFR-1) as well as several PKC isotypes; PTK787 inhibits KDR and Flk-1 (human and murine VEGFR-2, respectively), VEGFR-1, PDGFR-β, c-Kit, and cFms; and MAE 87 inhibits VEGFR-2, IGF-1R, FGFR-1, and EGFR. Inhibition of multiple kinases may completely block neovascularization, however, such inhibition is expected to have toxic side effects.

The present invention addresses the above-cited ocular pathologies and provides compositions and methods using interfering RNAs that target a select set of receptor tyrosine kinases involved in signal transduction pathways for treating neovascularization in retinal edema, diabetic retinopathy, sequela associated with retinal ischemia, and posterior segment neovascularization, for example.

### SUMMARY OF THE INVENTION

The present invention overcomes these and other drawbacks of the prior art by providing highly potent and efficacious prevention or intervention of pathologic ocular neovascularization-related conditions. In certain embodiments, regression of posterior segment neovascularization-related conditions is induced. In one aspect, the methods of the invention include treating such an ocular neovascularization related condition by administering interfering RNAs that silence expression of a select group of ocular RTK target mRNAs involved in an ocular neovascularization-related condition, thus decreasing signal transduction of downstream processes and treating ocular neovascularization and related conditions by effecting a lowering of ocular pre-angiogenic and angiogenic cellular activity. The select group of ocular RTK target mRNAs includes KDR (VEGFR-2) and at least one of Tie-2, PDGFRA, PDGFRB, FLT1 (VEGFR-1), KIT, CSF1R, FLT3, FLT4 (VEGFR-3) variant 1 and FLT4 (VEGFR-3) variant 2 mRNAs.

The term “an ocular neovascularization-related condition,” as used herein, includes ocular pre-angiogenic conditions and ocular angiogenic conditions, and includes those cellular changes resulting from the expression of select RTK-mRNAs that lead directly or indirectly to ocular neovasularization and related conditions. The interfering RNAs of the invention are useful for treating patients with ocular NV, abnormal angiogenesis, retinal vascular permeability, retinal edema, diabetic retinopathy particularly proliferative diabetic retinopathy, diabetic macular edema, exudative age-related macular degeneration, sequela associated with retinal ischemia, and posterior segment neovascularization, or patients at risk of developing such conditions, for example. The select set of RTK mRNAs provided herein provides for such silencing while avoiding toxic side effects due to nonspecific silencing of multiple kinases.

Embodiments of the present invention include a method of attenuating expression of a first and a second RTK mRNA in a subject, and a method of treating an ocular neovascularization-related condition in a subject in need thereof. The first mRNA is KDR (VEGFR-2) mRNA and the second RTK mRNA is any one of Tie-2, PDGFRA, PDGFRB, FLT1, KIT, CSF1R, FLT3, FLT4, FLT4 variant 1 and FLT4 variant 2 mRNA. The method comprises administering to the subject a composition comprising an effective amount of at least a first and a second interfering RNA and a pharmaceutically acceptable carrier, each interfering RNA having a length of 19 to 49 nucleotides. Administration is to the eye of the subject for attenuating expression of an ocular neovascularization-related condition target in a human.

In one embodiment, the first RTK mRNA is KDR (VEGFR-2) mRNA and a second RTK mRNA is Tie-2 mRNA. In another embodiment, the first RTK mRNA is KDR (VEGFR-2) mRNA and a second RTK mRNA is PDGFRA or PDGFRB mRNA. In yet another embodiment, the first RTK mRNA is KDR (VEGFR-2) mRNA, a second RTK mRNA is PDGFRA or PDGFRB mRNA, and a third RTK mRNA is FLT1 or Tie-2 mRNA. In yet another embodiment, the first mRNA is KDR (VEGFR-2) mRNA, a second RTK mRNA is PDGFRA or PDGFRB mRNA, a third RTK mRNA is FLT1 and a fourth RTK mRNA is Tie-2 mRNA. In a further embodiment, the first RTK mRNA is KDR (VEGFR-2) mRNA, a second RTK mRNA is FLT1 and a third RTK mRNA is Tie-2 mRNA. In further embodiments, a second, third, fourth, or fifth RTK mRNA is KIT mRNA, CSF1R mRNA, FLT3 mRNA, FLT4 mRNA, FLT4 variant 1 mRNA or FLT4 variant 2 mRNA.

In one embodiment, the first interfering RNA comprises a region of at least 13 contiguous nucleotides having at least 90% sequence complementarity to, or at least 90% sequence identity with, the penultimate 13 nucleotides of the 3′ end of an mRNA corresponding to any one of SEQ ID NO:16-SEQ ID NO:49 and SEQ ID NO:164-SEQ ID NO:170 which are target sequences of the KDR (VEGFR-2) cDNA, and the second interfering RNA comprises a region of at least 13 contiguous nucleotides having at least 90% sequence complementarity to, or at least 90% sequence identity with, the penultimate 13 nucleotides of the 3′ end of an mRNA corresponding to any one of SEQ ID NO:50-SEQ ID NO:89, SEQ ID NO:90-SEQ ID NO:124, SEQ ID NO:125-SEQ ID NO:163, SEQ ID NO:171-SEQ ID NO:174, SEQ ID NO:175-SEQ ID NO:189, SEQ ID NO:190-SEQ ID NO:204, and SEQ ID NO:211-SEQ ID NO:439, which are target sequences of the Tie-2, PDGFRA, PDGFRB, FLT1, KIT, CSF1R, FLT3, FLT4 variant 1 and FLT4 variant 2 cDNAs, as provided by Tables 2-8 infra.

In a further embodiment, the method comprises attenuating expression of a third RTK mRNA. For this embodiment, the composition further comprises a third interfering RNA comprising a region of at least 13 contiguous nucleotides having at least 90% sequence complementarity to, or at least 90% sequence identity with, the penultimate 13 nucleotides of the 3′ end of an mRNA corresponding to any one of SEQ ID NO:50-SEQ ID NO:89, SEQ ID NO:90-SEQ ID NO:124, SEQ ID NO:125-SEQ ID NO:163, SEQ ID NO:171-SEQ ID NO:174, SEQ ID NO:175-SEQ ID NO:189, SEQ ID NO:190-SEQ ID NO:204, and SEQ ID NO:211-SEQ ID NO:439, which region is not targeted by the second interfering RNA.

In further embodiments of the above-cited methods of the present invention, the region of contiguous nucleotides is a region of at least 14 contiguous nucleotides having at least 85% sequence complementarity to, or at least 85% sequence identity with, the penultimate 14 nucleotides of the 3′ end of the sequence of the target sequence identifier. In yet another embodiment of the invention, the region of contiguous nucleotides is a region of at least 15, 16, 17, or 18 contiguous nucleotides having at least 80% sequence complementarity to, or at least 80% sequence identity with, the penultimate 15, 16, 17, or 18 nucleotides, respectively, of the 3′ end of the sequence of the target sequence identifier.

Further embodiments of the invention include a method of attenuating expression of a first and a second RTK mRNA in a subject, and a method of treating an ocular neovascularization-related condition in a subject in need thereof. The methods comprise administering to the subject a composition comprising an effective amount of a first and a second interfering RNA and a pharmaceutically acceptable carrier. Administering is to an eye of the subject for treating an ocular neovascularization-related condition. Each interfering RNA has a length of 19 to 49 nucleotides and comprises a sense nucleotide strand, an antisense nucleotide strand, and a region of at least near-perfect contiguous complementarity of at least 19 nucleotides. The antisense strand of the first interfering RNA hybridizes under physiological conditions to a portion of mRNA corresponding to SEQ ID NO:1, and has a region of at least near-perfect contiguous complementarity of at least 19 nucleotides with the hybridizing portion of mRNA corresponding to SEQ ID NO:1, and the antisense strand of the second interfering RNA hybridizes under physiological conditions to a portion of mRNA corresponding to SEQ ID NO:2, SEQ ID NO:3, SEQ ID NO:4, SEQ ID NO:205, SEQ ID NO:206, SEQ ID NO:207, SEQ ID NO:208, SEQ ID NO:209 or SEQ ID NO:210, and has a region of at least near-perfect contiguous complementarity of at least 19 nucleotides with the hybridizing portion of mRNA corresponding to SEQ ID NO:2, SEQ ID NO:3, SEQ ID NO:4, SEQ ID NO:205, SEQ ID NO:206, SEQ ID NO:207, SEQ ID NO:208, SEQ ID NO:209 or SEQ ID NO:210, respectively. The expression of the first and second RTK mRNA is attenuated thereby.

In further embodiments of the methods of the above cited paragraph:
the antisense strand of the first interfering RNA is designed to target an mRNA corresponding to SEQ ID NO:1 comprising nucleotide 922, 942, 990, 1044, 1104, 1169, 1442, 2432, 2742, 2753, 2961, 3065, 3355, 3401, 3414, 3784, 3785, 3825, 4085, 4290, 4356, 4453, 4476, 4515, 4525, 4737, 4863, 4868, 4880, 5042, 5057, 5172, 5650, 5764, 1118, 1609, 1890, 2151, 2323, 2639, or 2654; and
the antisense strand of the second interfering RNA is designed to target an mRNA corresponding to SEQ ID NO:2 comprising nucleotide 432, 521, 712, 1273, 1276, 1455, 1467, 1581, 1582, 1809, 1830, 1904, 1905, 1937, 1938, 1945, 2114, 2138, 2153, 2154, 2197, 2199, 2610, 3002, 3165, 3348, 3408, 3410, 3443, 3603, 3624, 3626, 3633, 3645, 3799, 3918, 3974, 4051, 4053, 4110, 923, 1213, 1225, or 1269; or
the antisense strand of the second interfering RNA is designed to target an mRNA corresponding to SEQ ID NO:3 comprising nucleotide 489, 537, 574, 613, 1262, 1267, 1268, 1269, 1307, 1315, 1465, 1644, 1976, 2297, 3556, 3842, 4004, 4080, 4257, 4414, 4416, 4430, 4659, 4660, 4692, 4969, 4999, 5000, 5259, 5284, 5341, 5355, 5433, 5750, 6115, 587, 615, 918, 921, 1129, 1478, 2073, 2435, 2436, 2922, 2946, 3203, 3348, 3366, or 3387; or
the antisense strand of the second interfering RNA is designed to target an mRNA corresponding to SEQ ID NO:4 comprising nucleotide 807, 808, 860, 878, 885, 905, 939, 1065, 1197, 1347, 1692, 2352, 2845, 2958, 3635, 3954, 4162, 4391, 4742, 4774, 4780, 4882, 5183, 5184, 5478, 5480, 5538, 5540, 5542, 5543, 5544, 5546, 5547, 5548, 5562, 5563, 5567, 5591, 5697, 1896, 2193, 2340, 2362, 2363, 2538, 2740, 2747, 2760, 2829, 2926, 3030, 3031, 3192, or 3252; or
the antisense strand of the second interfering RNA is designed to target an mRNA corresponding to SEQ ID NO:205 comprising nucleotide 437, 464, 577, 647, 1171, 1198, 1215, 1304, 1305, 1343, 1402, 1460, 1497, 1766, 1767, 2044, 2478, 2560, 2623, 2624, 2779, 2780, 2963, 2990, 3002, 3453, 3615, 3616, 3769, 4064, 4065, 4229, 4465, 4493, 4565, 4708, 5097, 5147, 5372, 5484, 5486, 5487, 5495, 5496, 5568, 5569, or 5726; or
the antisense strand of the second interfering RNA is designed to target an mRNA corresponding to SEQ ID NO:206 comprising nucleotide 174, 319, 708, 709, 710, 776, 812, 852, 1058, 1075, 1076, 1077, 1150, 1203, 1435, 1478, 1619, 1637, 1640, 1694, 1746, 1837, 1839, 2423, 2425, 2496, 2634, 2642, 2645, 2875, 3135, 3136, 3278, 3332, 3371, 3464, 3616, 3670, 3726, 3843, 3979, 4142, 4218, 4319, 4320, 4377, 4515, 4568, or 5000; or
the antisense strand of the second interfering RNA is designed to target an mRNA corresponding to SEQ ID NO:207 comprising nucleotide 969, 1078, 1080, 1081, 1101, 1179, 1458, 1505, 1509, 1705, 1890, 1893, 1895, 1944, 2292, 2303, 2375, 2388, 2391, 2591, 2637, 2875, 2883, 3303, 3305, 3470, 3477, 3487, 3493, 3495, 3526, 3532, 3533, 3535, 3536, 3688, 3690, 3776, 3796, 3914, or 3915; or
the antisense strand of the second interfering RNA is designed to target an mRNA corresponding to SEQ ID NO:208 comprising nucleotide 494, 683, 684, 817, 819, 820, 1079, 1115, 1249, 1591, 1736, 1798, 1799, 1827, 1828, 1829, 1970, 1971, 2148, 2276, 2391, 2547, 2565, 2641, 2735, 2903, 2941, 3028, 3081, 3082, 3085, 3169, 3170, 3171, 3178, 3299, 3309, 3316, 3317, 3365, or 3371; or
the antisense strand of the second interfering RNA is designed to target an mRNA corresponding to SEQ ID NO:209 comprising nucleotide 84, 343, 409, 420, 424, 427, 691, 694, 943, 1060, 1111, 1317, 1456, 1690, 1709, 1752, 1834, 2005, 2191, 2341, 2608, 2695, 2697, 3160, 3268, 3451, 3878, 3884, 4199, 4201, 4355, 4418, 4419, 4420, 4421, 4503, 4506, 4511, 4674, 4675, 4722, or 4724; or
the antisense strand of the second interfering RNA is designed to target an mRNA corresponding to SEQ ID NO:210 comprising nucleotide 4002, 4009, 4293, 4314, 4330, 4375, 4376, 4377, or 4385.

In another embodiment of the above cited methods, a third interfering RNA may be included in the composition for targeting a third RTK mRNA, i.e., either Tie-2, PDGFRA, PDGFRB, FLT1, KIT, CSF1R, FLT3, FLT4, FLT4 variant 1 or FLT4 variant 2 not targeted by the second interfering RNA. The method comprises administering to the subject a third interfering RNA having a length of 19 to 49 nucleotides and comprising a sense nucleotide strand, an antisense nucleotide strand, and a region of at least near-perfect complementarity of at least 19 nucleotides. The antisense strand of the third interfering RNA hybridizes under physiological conditions to a portion of mRNA corresponding to SEQ ID NO:2, SEQ ID NO:3, SEQ ID NO:4, SEQ ID NO:205, SEQ ID NO:206, SEQ ID NO:207, SEQ ID NO:208, SEQ ID NO:209 or SEQ ID NO:210 and the antisense strand has a region of at least near-perfect contiguous complementarity of at least 19 nucleotides with the hybridizing portion of mRNA corresponding to SEQ ID NO:2, SEQ ID NO:3, SEQ ID NO:4, SEQ ID NO:205, SEQ ID NO:206, SEQ ID NO:207, SEQ ID NO:208, SEQ ID NO:209 or SEQ ID NO:210, respectively.

Another embodiment of the invention is a method of attenuating expression of a first and a second ocular neovascularization-related condition target mRNA of a subject, comprising administering to the subject a composition comprising an effective amount of a first and a second single-stranded interfering RNA, each interfering RNA having a length of 19 to 49 nucleotides, and a pharmaceutically acceptable carrier. The first single-stranded interfering RNA hybridizes under physiological conditions to a portion of mRNA corresponding to SEQ ID NO:1 and the second single-stranded interfering RNA hybridizes under physiological conditions to a portion of mRNA corresponding to SEQ ID NO:2, SEQ ID NO:3, SEQ ID NO:4, SEQ ID NO:205, SEQ ID NO:206, SEQ ID NO:207, SEQ ID NO:208, SEQ ID NO:209 or SEQ ID NO:210; the hybridizing portions of mRNA identified by nucleotide positions cited supra for antisense strands.

A composition comprising a first and a second interfering RNA, each interfering RNA having a length of 19 to 49 nucleotides, wherein the first interfering RNA comprises a nucleotide sequence of any one of SEQ ID NO:16-SEQ ID NO:49 and SEQ ID NO:164-SEQ ID NO:170, or a complement thereof; and the second interfering RNA comprises a nucleotide sequence of any one of SEQ ID NO:50-SEQ ID NO:89, SEQ ID NO:90-SEQ ID NO:124, SEQ ID NO:125-SEQ ID NO:163, SEQ ID NO:171-SEQ ID NO:174, SEQ ID NO:175-SEQ ID NO:189, SEQ ID NO:190-SEQ ID NO:204, and SEQ ID NO:211-SEQ ID NO:439, or a complement thereof, and a pharmaceutically acceptable carrier is an embodiment of the present invention. In one embodiment, the interfering RNA is isolated. The term “isolated” means that the interfering RNA is free of its total natural mileau.

A method of attenuating expression of an ocular neovascularization-related condition target mRNA first variant without attenuating expression of an ocular neovascularization-related condition target mRNA second variant in a subject is a further embodiment of the invention. The method comprises administering to the subject a composition comprising an effective amount of interfering RNA having a length of 19 to 49 nucleotides and a pharmaceutically acceptable carrier, the interfering RNA comprising a region of at least 13 contiguous nucleotides having at least 90% sequence complementarity to, or at least 90% sequence identity with, the penultimate 13 nucleotides of the 3′ end of the first variant, wherein the expression of the first variant mRNA is attenuated without attenuating expression of the second variant mRNA, and wherein the first variant target mRNA is SEQ ID NO:209, and the second variant target mRNA is SEQ ID NO:210.

In a further embodiment of the above-cited method, the first variant target mRNA is SEQ ID NO:210, and the second variant target mRNA is SEQ ID NO:209.

Use of any of the embodiments as described herein in the preparation of a medicament for attenuating expression of the select RTK ocular neovascularization-related condition target mRNAs as set forth herein is also an embodiment of the present invention.

### BRIEF DESCRIPTION OF THE FIGURES

In order that the manner in which the above-recited and other enhancements and objects of the invention are obtained, a more particular description of the invention briefly described above will be rendered by reference to specific embodiments thereof which are illustrated in the appended drawings. Understanding that these drawings depict only typical embodiments of the invention and are therefore not to be considered limiting of its scope, the invention will be described with additional specificity and detail through the use of the accompanying drawings in which:

FIG. 1 shows provides a KDR (VEGFR2) western blot of bEnd.3 cells transfected with KDR siRNAs #1, #2, #3, and #4 at 0.1-10 nM. The arrows indicate the positions of the ˜220-kDa KDR and 42-kDa actin bands.

FIG. 2 provides a TIE2 (TEK) western blot of bEnd.3 cells transfected with a TIE2 siRNA, a non-targeting control siRNA (NTC2), and a RISC-free control siRNA at 100 nM; and a buffer control (-siRNA). The arrows indicate the positions of the 140-kDa TIE2 and 42-kDa actin bands.

FIG. 3 provides KDR (VEGFR2) and TIE2 (TEK) western blots of bEnd.3 cells transfected with a KDR siRNA at 10 nM, a TIE2 siRNA at 10 nM, mixtures of the KDR and TIE2 siRNAs at 1-10 nM, and a non-targeting control siRNA (NTC2) at 10 nM. The arrows indicate the positions of the ˜220-kDa KDR, 140-kD TIE2, and 42-kDa actin bands.

### DETAILED DESCRIPTION OF THE INVENTION

As used herein and unless otherwise indicated, the terms “a” and “an” are taken to mean “one,” “at least one” or “one or more.”

In the following description, specific details are set forth such as specific quantities, sizes, etc. so as to provide a thorough understanding of embodiments of the present invention. However, it will be obvious to those skilled in the art that the present invention may be practiced without such specific details. In many cases, details concerning such considerations and the like have been omitted inasmuch as such details are not necessary to obtain a complete understanding of the present invention and are within the skills of persons of ordinary skill in the relevant art.

Referring to the drawings in general, it will be understood that the illustrations are for the purpose of describing a particular embodiment of the invention and are not intended to limit the invention thereto.

While most of the terms used herein will be recognizable to those of skill in the art, the following definitions are nevertheless put forth to aid in the understanding of the present invention. It should be understood, however, that when not explicitly defined, terms should be interpreted as adopting a meaning presently accepted by those of skill in the art. If resort to an outside source is necessary because an interpretation from the intrinsic evidence would render one or more claims invalid Webster's New World Dictionary, 3^{rd} Edition should be used.

The following description uses the terms agent(s) and compound(s) interchangeably, unless otherwise indicated. Further, the following description uses the terms formulation(s) and medicament(s) interchangeably, unless otherwise indicated.

The phrase “a region of at least 13 contiguous nucleotides having at least 90% sequence complementarity to, or at least 90% sequence identity with, the penultimate 13 nucleotides of the 3′ end of an mRNA corresponding to any one of (a sequence identifier)” allows a one nucleotide substitution. Two nucleotide substitutions (i.e., 11/13=85% identity/complementarity) are not included in such a phrase.

As used herein, the term “hybridization” means and refers to a process in which single-stranded nucleic acids with complementary or near-complementary base sequences interact to form hydrogen-bonded complexes called hybrids. Hybridization reactions are sensitive and selective. In vitro, the specificity of hybridization (i.e., stringency) is controlled by the concentrations of salt or formamide in prehybridization and hybridization solutions, for example, and by the hybridization temperature; such procedures are well known in the art. In particular, stringency is increased by reducing the concentration of salt, increasing the concentration of formamide, or raising the hybridization temperature.

As used herein, the term “percent identity” describes the percentage of contiguous nucleotides in a first nucleic acid molecule that is the same as in a set of contiguous nucleotides of the same length in a second nucleic acid molecule. The term “percent complementarity” describes the percentage of contiguous nucleotides in a first nucleic acid molecule that can base pair in the Watson-Crick sense with a set of contiguous nucleotides in a second nucleic acid molecule.

As used herein, the term “siRNA” as used herein refers to a double-stranded interfering RNA unless otherwise noted.

Nucleic acid sequences cited herein are written in a 5′ to 3′ direction unless indicated otherwise. The term “nucleic acid,” as used herein, refers to either DNA or RNA or a modified form thereof comprising the purine or pyrimidine bases present in DNA (adenine “A,” cytosine “C,” guanine “G,” thymine “T”) or in RNA (adenine “A,” cytosine “C,” guanine “G,” uracil “U”). Interfering RNAs provided herein may comprise “T” bases, particularly at 3′ ends, even though “T” bases do not naturally occur in RNA. “Nucleic acid” includes the terms “oligonucleotide” and “polynucleotide” and can refer to a single-stranded molecule or a double-stranded molecule. A double-stranded molecule is formed by Watson-Crick base pairing between A and T bases, C and G bases, and between A and U bases. The strands of a double-stranded molecule may have partial, substantial or full complementarity to each other and will form a duplex hybrid, the strength of bonding of which is dependent upon the nature and degree of complementarity of the sequence of bases.

The relationship between a target mRNA (sense strand) and one strand of an siRNA (the sense strand) is that of identity. The sense strand of an siRNA is also called a passenger strand, if present. The relationship between a target mRNA (sense strand) and the other strand of an siRNA (the antisense strand) is that of complementarity. The antisense strand of an siRNA is also called a guide strand.

The penultimate base in a nucleic acid sequence that is written in a 5′ to 3′ direction is the next to the last base, i.e., the base next to the 3′ base. The penultimate 13 bases of a nucleic acid sequence written in a 5′ to 3′ direction are the last 13 bases of a sequence next to the 3′ base and not including the 3′ base. Similarly, the penultimate 14, 15, 16, 17, or 18 bases of a nucleic acid sequence written in a 5′ to 3′ direction are the last 14, 15, 16, 17, or 18 bases of a sequence, respectively, next to the 3′ base and not including the 3′ base.

An mRNA sequence is readily deduced from the sequence of the corresponding DNA sequence. For example, SEQ ID NO:1 provides the sense strand sequence of DNA corresponding to the mRNA for AQP1, variant a. The mRNA sequence is identical to the DNA sense strand sequence with the “T” bases replaced with “U” bases. Therefore, the mRNA sequence of AQP1, variant a, is known from SEQ ID NO:1, for example.

As used herein, the term “health care provider” and/or “clinician is known in the art and specifically includes a physician, a person with authority to prescribe a medication (whether directly or indirectly), and a veterinarian. In certain embodiments, a health care provider includes an individual that provides a medication without prescription, such as in providing an over-the-counter medication.

As used herein, the terms “identifying subjects” and “diagnosing” are used interchangeably with regard to the detection of a “predisposition,” “increased propensity,” “risk,” “increased risk,” and the like.

As used herein, the term “ocular disease” means and refers to at least one of age-related macular degeneration, cataract, acute ischemic optic neuropathy (AION), commotio retinae, retinal detachment, retinal tears or holes, diabetic retinopathy and iatrogenic retinopathy and other ischemic retinopathies or optic neuropathies, myopia, retinitis pigmentosa, and/or the like.

As used herein, the term “pharmaceutically acceptable,” such as in the recitation of a “pharmaceutically acceptable carrier,” or a “pharmaceutically acceptable acid addition salt,” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. “Pharmacologically active” (or simply “active”) as in a “pharmacologically active” derivative or metabolite, refers to a derivative or metabolite having the same type of pharmacological activity as the parent compound and approximately equivalent in degree. When the term “pharmaceutically acceptable” is used to refer to a derivative (e.g., a salt) of an active agent, it is to be understood that the compound is pharmacologically active as well.

As used herein, a “pharmaceutically acceptable salt” or “salt” means and refers to a salt of a phosphodiesterase inhibitor that retains the function of the inhibitor and that is compatible with administration as desired. A salt may be formed from an acid or a base depending upon the nature of the antagonist. A salt may be formed from an acid such as acetic acid, benzoic acid, cinnamic acid, citric acid, ethanesulfonic acid, fumaric acid, glycolic acid, hydrobromic acid, hydrochloric acid, maleic acid, malonic acid, mandelic acid, methanesulfonic acid, nitric acid, oxalic acid, phosphoric acid, propionic acid, pyruvic acid, salicylic acid, succinic acid, sulfuric acid, tartaric acid, p-toluenesulfonic acid, trifluoroacetic acid, and the like. A salt may be formed with a base such as a primary, secondary, or tertiary amine, or from cation sources such as those derived from aluminum, ammonium, calcium, copper, iron, lithium, magnesium, manganese, potassium, sodium, zinc, and the like.

As used herein, the term “subject” or “patient” refers to any invertebrate or vertebrate species. The methods of the present invention are particularly useful in the treatment of warm-blooded vertebrates. Thus, in an embodiment, the invention concerns mammals and birds.

As used herein, “therapeutically effective amount” or “effective amount” means and refers to the amount of the PDE inhibitor and/or other active agent that is effective to achieve its intended purpose. It is desired, but not required that the effective amount not cause extreme and/or severe undesirable side effects. While individual patient and/or subject needs may vary, determination of optimal ranges for effective amounts of each of the compounds and compositions is within the skill of the art. Generally, the dosage required to provide an effective amount of the composition, and which can be adjusted by one of ordinary skill in the art will vary, depending on the age, health, physical condition, sex, weight, frequency of treatment and the nature and scope of the disorder.

As used herein, “inhibitor” is used to describe modulating effects of phosphodiesterase (PDE) inhibitors. PDE inhibitors may inhibit the biosynthesis of cAMP and/or cGMP.

RNA interference (RNAi) is a process by which double-stranded RNA (dsRNA) is used to silence gene expression. While not wanting to be bound by theory, RNAi begins with the cleavage of longer dsRNAs into small interfering RNAs (siRNAs) by an RNaseIII-like enzyme, dicer. SiRNAs are dsRNAs that are usually about 19 to 28 nucleotides, or 20 to 25 nucleotides, or 21 to 22 nucleotides in length and often contain 2-nucleotide 3′ overhangs, and 5′ phosphate and 3′ hydroxyl termini. One strand of the siRNA is incorporated into a ribonucleoprotein complex known as the RNA-induced silencing complex (RISC). RISC uses this siRNA strand to identify mRNA molecules that are at least partially complementary to the incorporated siRNA strand, and then cleaves these target mRNAs or inhibits their translation. Therefore, the siRNA strand that is incorporated into RISC is known as the guide strand or the antisense strand. The other siRNA strand, known as the passenger strand or the sense strand, is eliminated from the siRNA and is at least partially homologous to the target mRNA. Those of skill in the art will recognize that, in principle, either strand of an siRNA can be incorporated into RISC and function as a guide strand. However, siRNA design (e.g., decreased siRNA duplex stability at the 5′ end of the antisense strand) can favor incorporation of the antisense strand into RISC.

RISC-mediated cleavage of mRNAs having a sequence at least partially complementary to the guide strand leads to a decrease in the steady state level of that mRNA and of the corresponding protein encoded by this mRNA. Alternatively, RISC can also decrease expression of the corresponding protein via translational repression without cleavage of the target mRNA. Other RNA molecules and RNA-like molecules can also interact with RISC and silence gene expression. Examples of other RNA molecules that can interact with RISC include short hairpin RNAs (shRNAs), single-stranded siRNAs, microRNAs (miRNAs), and dicer-substrate 27-mer duplexes. The term “siRNA” as used herein refers to a double-stranded interfering RNA unless otherwise noted. Examples of RNA-like molecules that can interact with RISC include RNA molecules containing one or more chemically modified nucleotides, one or more deoxyribonucleotides, and/or one or more non-phosphodiester linkages. For purposes of the present discussion, all RNA or RNA-like molecules that can interact with RISC and participate in RISC-mediated changes in gene expression will be referred to as “interfering RNAs.” SiRNAs, shRNAs, miRNAs, and dicer-substrate 27-mer duplexes are, therefore, subsets of “interfering RNAs.”

Interfering RNA of embodiments of the invention appear to act in a catalytic manner for cleavage of target mRNA, i.e., interfering RNA is able to effect inhibition of target mRNA in substoichiometric amounts. As compared to antisense therapies, significantly less interfering RNA is required to provide a therapeutic effect under such cleavage conditions.

The present invention relates to the use of interfering RNA to inhibit the expression of select RTK target mRNAs, thus inhibiting neovascularization-related conditions, thereby preventing or treating pathologic ocular neovascularization, retinal edema, diabetic retinopathy, and sequela associated with retinal ischemia, as well as induce the regression of posterior segment neovascularization (PSNV). Select RTK target mRNAs include KDR (kinase insert domain-containing receptor; also known as VEGFR-2) mRNA and at least a second RTK mRNA such as TIE-2 (also known as TEK, TIE2, VMCM, VMCM1, or CD202B) mRNA, PDGFRA (platelet derived growth factor receptor alpha subunit) mRNA, or PDGFRB (platelet derived growth factor receptor beta subunit) mRNA, FLT1 (VEGFR-1) mRNA, KIT mRNA, CSF1R mRNA, FLT3 mRNA, and FLT4 (VEGFR-3) variant 1 and FLT4 (VEGFR-3) variant 2 mRNAs. According to the present invention, such a combination of interfering RNAs provided exogenously or expressed endogenously are particularly effective at silencing such select RTK target mRNAs in ocular tissue(s) without causing toxic side effects seen when inhibiting multiple kinases using small molecules.

The reversible phosphorylation of proteins is one of the primary biochemical mechanisms mediating eukaryotic cell signaling. This reaction is catalyzed by protein kinases that transfer the gamma phosphate group of ATP to hydroxyl groups on target proteins. Human tyrosine kinases have been organized in dendrogram format based on the sequence homology of their catalytic domains (CITE). Cytosolic tyrosine kinases reside intracellularly, whereas receptor tyrosine kinases (RTKs) possess both extracellular and intracellular domains and function as membrane spanning cell surface receptors. As such, RTKs mediate the cellular responses to environmental signals and facilitate a broad range of cellular processes including proliferation, migration and survival.

Since RTKs are one of the principal components of the signaling network that transmits extracellular signals into cells, RTK dysregulation of signaling pathways is associated with a variety of human disorders including ocular disease. The present invention provides for inhibition of a select set of RTK mRNAs for inhibition and regression of ocular neovascularization-related conditions.

Several RTKs, including KIT, CSF-1R, and FLT3 (infra), are expressed by hematopoietic precursor cells (HPCs) and appear to be involved in pathologic ocular angiogenesis. For example, HPCs have been shown to migrate to sites of choroidal neovascularization. However, the majority of data related to these RTKs has been generated in oncology models.

Nucleic acid sequences cited herein are written in a 5′ to 3′ direction unless indicated otherwise. The term “nucleic acid,” as used herein, refers to either DNA or RNA or a modified form thereof comprising the purine or pyrimidine bases present in DNA (adenine “A,” cytosine “C,” guanine “G,” thymine “T”) or in RNA (adenine “A,” cytosine “C,” guanine “G,” uracil “U”). Interfering RNAs provided herein may comprise “T” bases, particularly at 3′ ends, even though “T” bases do not naturally occur in RNA. “Nucleic acid” includes the terms “oligonucleotide” and “polynucleotide” and can refer to a single-stranded molecule or a double-stranded molecule. A double-stranded molecule is formed by Watson-Crick base pairing between A and T bases, C and G bases, and between A and U bases. The strands of a double-stranded molecule may have partial, substantial or full complementarity to each other and will form a duplex hybrid, the strength of bonding of which is dependent upon the nature and degree of complementarity of the sequence of bases.

An mRNA sequence is readily deduced from the sequence of the corresponding DNA sequence. For example, SEQ ID NO:1 provides the sense strand sequence of DNA corresponding to the mRNA for KDR (VEGFR-2). The mRNA sequence is identical to the DNA sense strand sequence with the “T” bases replaced with “U” bases.

Therefore, the mRNA sequence for KDR (VEGFR-2) is known from SEQ ID NO:1, the mRNA sequence for TIE-2 is known from SEQ ID NO:2, the mRNA sequence for PDGFRA is known from SEQ ID NO:3, the mRNA sequence for PDGFRB is known from SEQ ID NO:4, the mRNA sequence for FLT1 (VEGFR-1) is known from SEQ ID NO:205, the mRNA sequence for KIT is known from SEQ ID NO:206, the mRNA sequence for CSF1R is known from SEQ ID NO:207, the mRNA sequence for FLT3 is known from SEQ ID NO:208, and the mRNA sequences for FLT4 variant 1 and FLT4 variant 2 mRNAs are known from SEQ ID NO:209 and SEQ ID NO:210, respectively.

KDR Receptor Tyrosine Kinase (VEGFR-2, Flk-1):

KDR, also known as VEGFR-2 or Flk-1 (the murine homolog), is an RTK having ligands that include VEGF-A (VEGF), VEGF-C, and VEGF-D. Ligand binding induces receptor dimerization leading to autophosphorylation of several intracellular tyrosine residues and activation of several intracellular signaling pathways including the Raf-Mek-Erk pathway. KDR (VEGFR-2) is the major mediator of the mitogenic, angiogenic, and permeability-enhancing effects of VEGF. Signaling through KDR (VEGFR-2) appears to play a role in developmental angiogenesis. For example, Flk-1 null mice fail to develop organized blood vessels and die in utero at an early stage. Regarding vascular development in the retina, induction of VEGF leads to neovascularization in the posterior segment and breakdown of the blood-retinal barrier under pathological conditions. Furthermore, VEGF and the VEGF-R5 have been localized to neovascular tissues in patients with diabetic retinopathy and exudative AMD.

The GenBank database of the National Center for Biotechnology Information at ncbi.nlm.nih.gov provides the corresponding DNA sequence for the messenger RNA of KDR (VEGFR-2) tyrosine kinase as reference no. NM_{—}002253, provided in the “Sequence Listing” as SEQ ID NO:1. The coding sequence for KDR (VEGFR-2) is from nucleotides 304-4374.

Equivalents of the above-cited KDR (VEGFR-2) mRNA sequence are alternative splice forms, allelic forms, isozymes, or a cognate thereof. A cognate is a KDR (VEGFR-2) mRNA from another mammalian species that is homologous to SEQ ID NO:1 (i.e., an ortholog). KDR (VEGFR-2) nucleic acid sequences related to SEQ ID NO:1 include those having GenBank accession numbers AF035121, X61656, L04947, AF063658, and X89776.

Tie-2 Receptor Tyrosine Kinase (Tie-2):

Tie-2 (also known as TEK, Tie2, VMCM, VMCM1, or CD202B) is an RTK having as ligands the four members of the angiopoietin family, Ang1, Ang2, Ang3, and Ang4. Ang/Tie-2 signal transduction plays a role in vascular development and angiogenesis, both physiologic and pathologic. Ang/Tie-2 signaling appears to work in concert with the VEGF pathway during vasculo/angiogenesis. Ang2/Tie-2 signaling has been shown to mediate both pathologic ocular angiogenesis and retinal vascular permeability in rodent species. Moreover, homozygous knockout of Ang2 in diabetic mice has a protective effect against the morphologic vascular changes typical of nonproliferative diabetic retinopathy (DR).

The GenBank database of the National Center for Biotechnology Information at ncbi.nlm.nih.gov provides the corresponding DNA sequence for the messenger RNA of TEK tyrosine kinase as reference no. NM_{—}000459, provided in the “Sequence Listing” as SEQ ID NO:2. The coding sequence for TEK (TIE-2) is from nucleotides 149-3523.

Equivalents of the above-cited Tie-2 mRNA sequence are alternative splice forms, allelic forms, isozymes, or a cognate thereof. A cognate is a Tie-2 mRNA from another mammalian species that is homologous to SEQ ID NO:2 (i.e., an ortholog). Tie-2 nucleic acid sequences related to SEQ ID NO:2 include those having GenBank accession numbers L06139, AB208796, BC035514, AB086825, and U53603.

Platelet Derived Growth Factor Receptor Tyrosine Kinase (PDGFRA and PDGFRB):

PDGFR is an RTK expressed on the surface of fibroblasts, smooth muscle cells, and vascular endothelial cells. Two PDGFR subunits, α and β, are encoded by PDGFRA and PDGFRB, respectively. Ligand binding triggers homo- or heterodimerization of two PDGFR subunits resulting in activation of the tyrosine kinase. The PDGF/PDGFR pathway interacts with the VEGF/VEGFR pathway to regulate angiogenesis in the retina and other tissues.

The GenBank database of the National Center for Biotechnology Information at ncbi.nlm.nih.gov provides the corresponding DNA sequence for the messenger RNA of PDGFRA as reference no. NM_{—}006206, provided in the “Sequence Listing” as SEQ ID NO:3. The coding sequence for PDGFRA is from nucleotides 149-3418.

Equivalents of the above-cited PDGFRA mRNA sequence are alternative splice forms, allelic forms, isozymes, or a cognate thereof. A cognate is a PDGFRA mRNA from another mammalian species that is homologous to SEQ ID NO:3 (i.e., an ortholog). PDGFRA nucleic acid sequences related to SEQ ID NO:3 include those having GenBank accession numbers M21574, M22734, BC063414, BC015186, X76079, L25829, D50017, AJ278993, X80389, and M30494.

The GenBank database of the National Center for Biotechnology Information at ncbi.nlm.nih.gov provides the corresponding DNA sequence for the messenger RNA of PDGFRB as reference no. NM_{—}002609, provided in the “Sequence Listing” as SEQ ID NO:4. The coding sequence for PDGFRB is from nucleotides 470-3790.

Equivalents of the above-cited PDGFRB mRNA sequence are alternative splice forms, allelic forms, isozymes, or a cognate thereof. A cognate is a PDGFRB mRNA from another mammalian species that is homologous to SEQ ID NO:4 (i.e., an ortholog). PDGFRB nucleic acid sequences related to SEQ ID NO:4 include those having GenBank accession numbers BC032224, J03278, M21616, AB209657, and M30493.

FLT1 (FMS-like tyrosine kinase, VEGFR-1):

FLT1 is an RTK that binds VEGF-A, a KDR ligand, as well as VEGF-B and P1GF (placenta growth factor), two non-KDR ligands. The role of FLT1 in signal transduction appears to differ depending on developmental stage and/or cell type. Ligand binding initiates a weaker tyrosine phosphorylation in FLT1 than is observed with KDR, and activated FLT1 appears to interact with components of signal transduction pathways. For example, P1GF binding to FLT1 activates the PI3K/AKT and ERK pathways in monocytes. FLT1 may also be a “decoy” receptor that sequesters VEGF-A making it less available to KDR. A soluble form of FLT1, sFlt-1, may perform a similar function. P1GF binding to FLT1 may also result in transphoshorylation of KDR, thus amplifying VEGF/KDR signaling. Regardless of the mechanism, FLT1 clearly has a role in angiogenesis during development. For example, endothelial cells in FLT1^{-/-} mice fail to organize in vascular channels resulting in embryonic lethality.

The GenBank database of the National Center for Biotechnology Information at ncbi.nlm.nih.gov provides the corresponding DNA sequence for the messenger RNA of FLT1 (VEGFR-1) tyrosine kinase as reference no. NM_{—}002019, provided in the “Sequence Listing” as SEQ ID NO:205. The coding sequence for FLT1 is from nucleotides 250-4266.

Equivalents of the above cited FLT1 (VEGFR-1) mRNA sequence are alternative splice forms, allelic forms, isozymes, or a cognate thereof. A cognate is a FLT1 (VEGFR-1) mRNA from another mammalian species that is homologous to SEQ ID NO:205 (i.e., an ortholog). FLT1 (VEGFR-1) nucleic acid sequences related to SEQ ID NO:205 include those having GenBank accession numbers X51602, AF063657, BC039007, AB209050, BC029849, BC046165, BC048278, AF339822, and U01134.

KIT Receptor Tyrosine Kinase:

KIT, also known as c-Kit, is the homolog of the feline sarcoma virus oncogene, v-kit. KIT is an RTK belonging to the same subclass as PDGFR, FLT3, and CSF1R. KIT is expressed by HPCs, mast cells, germ cells and by pacemaker cells in the gut. Binding of either the soluble or membrane-associated forms of its ligand, stem cell factor or SCF, induces dimerization and autophospactivation of KIT. SCF acts primarily to promote survival of HPCs, however, it also induces chemotaxis and adhesions in certain cell populations. KIT signaling contributes to tumor progression through autocrine stimulation by SCF and through mutations that result in ligand-independent kinase activity.

The GenBank database of the National Center for Biotechnology Information at ncbi.nlm.nih.gov provides the corresponding DNA sequence for the messenger RNA of KIT tyrosine kinase as reference no. NM_{—}000222, provided in the “Sequence Listing” as SEQ ID NO:206. The coding sequence for KIT is from nucleotides 22-2952.

Equivalents of the above-cited KIT mRNA sequence are alternative splice forms, allelic forms, isozymes, or a cognate thereof. A cognate is a KIT mRNA from another mammalian species that is homologous to SEQ ID NO:206 (i.e., an ortholog). KIT nucleic acid sequences related to SEQ ID NO:206 include those having GenBank accession numbers X06182, BC071593, and AJ438313.

CSF1R Receptor Tyrosine Kinase: CSF1R is the cellular homolog of the retroviral oncogene v-fms. CSF1R is the receptor for colony stimulating factor 1 (CSF1). Like other RTKs, CSF binding to CSF1 stabilizes receptor dimerization resulting in autophosphorylation and leading to a series of downstream signaling events including cytoskeletal remodeling. CSF1 regulates the survival, proliferation and chemotaxis of macrophages and supports their activation, thus CSF1/CSF1R signaling is involved in the pathogenesis of several diseases.

The GenBank database of the National Center for Biotechnology Information at ncbi.nlm.nih.gov provides the corresponding DNA sequence for the messenger RNA of CSF1R tyrosine kinase as reference no. NM_{—}005211, provided in the “Sequence Listing” as SEQ ID NO:207. The coding sequence for CSF1R is from nucleotides 293-3211.

Equivalents of the above cited CSF1R mRNA sequence are alternative splice forms, allelic forms, isozymes, or a cognate thereof. A cognate is a CSF1R mRNA from another mammalian species that is homologous to SEQ ID NO:207 (i.e., an ortholog). CSF1R nucleic acid sequences related to SEQ ID NO:207 include those having GenBank accession numbers X03663 and BC047521.

FLT3 Receptor Tyrosine Kinase:

FLT3 is normally expressed on hematopoietic stem cells where its interaction with FLT3 ligand (FL) stimulates stem cell survival, proliferation and differentiation. In addition to being overexpressed in various leukemia cells, FLT3 is frequently mutated in hematological malignancies with approximately one-third of patients with acute myeloid leukemia (AML) harboring activating mutations.

The GenBank database of the National Center for Biotechnology Information at ncbi.nlm.nih.gov provides the corresponding DNA sequence for the messenger RNA of FLT3 tyrosine kinase as reference no. NM_{—}004119, provided in the “Sequence Listing” as SEQ ID NO:208. The coding sequence for FLT3 is from nucleotides 58-3039.

Equivalents of the above-cited FLT3 mRNA sequence are alternative splice forms, allelic forms, isozymes, or a cognate thereof. A cognate is a FLT3 mRNA from another mammalian species that is homologous to SEQ ID NO:208 (i.e., an ortholog). FLT3 nucleic acid sequences related to SEQ ID NO:208 include those having GenBank accession numbers U02687 and Z26652.

FLT4 Receptor Tyrosine Kinase variant 1 and variant 2 (VEGFR-3):

Despite its structural similarity to KDR and FLT1, the FLT4 (VEGFR-3) RTK mediates the signaling of VEGF-C and VEGF-D, but not of VEGF-A. FLT4 activation by at least one of its ligands provides for normal angiogenesis during development. FLT4^{-/-} mice fail to develop a normal vasculature and die early during embryonic development. In the adult, FLT4 is expressed on lymphatic endothelial cells but not on vascular endothelial cells. Therefore, although VEGF-C induces angiogenesis in the adult, this activity is thought to be mediated by KDR rather than FLT4. The role of FLT4 signaling under non-pathological conditions in the adult is likely limited to maintenance of lymphatic endothelial cells and/or lymphangiogenesis, however, FLT4 signaling may induce angiogenesis in tumors.

The GenBank database of the National Center for Biotechnology Information at ncbi.nlm.nih.gov provides the corresponding DNA sequence for the messenger RNA of FLT4 (VEGFR-3) tyrosine kinase, variant 1, as reference no. NM_{—}182925, provided in the “Sequence Listing” as SEQ ID NO:209. The coding sequence for FLT4 (VEGFR-3), variant 1, is from nucleotides 21-4112.

Equivalents of the above-cited FLT4 (VEGFR-3), variant 1, mRNA sequence are alternative splice forms, allelic forms, isozymes, or a cognate thereof. A cognate is a FLT4 (VEGFR-3) mRNA from another mammalian species that is homologous to SEQ ID NO:209 (i.e., an ortholog). FLT4 (VEGFR-3), variant 1, nucleic acid sequences related to SEQ ID NO:209 include those having GenBank accession numbers NM_{—}002020, AY233383, AY233382, X69878, U43143, and X68203.

The GenBank database of the National Center for Biotechnology Information at ncbi.nlm.nih.gov provides the corresponding DNA sequence for the messenger RNA of FLT4 (VEGFR-3) tyrosine kinase, variant 2, as reference no. NM_{—}002020, provided in the “Sequence Listing” as SEQ ID NO:210. The coding sequence for FLT4 (VEGFR-3), variant 2, is from nucleotides 22-3918.

Equivalents of the above-cited FLT4 (VEGFR-3), variant 2, mRNA sequence are alternative splice forms, allelic forms, isozymes, or a cognate thereof. A cognate is a FLT4 (VEGFR-3), variant 2, mRNA from another mammalian species that is homologous to SEQ ID NO:210 (i.e., an ortholog). FLT4 (VEGFR-3), variant 2, nucleic acid sequences related to SEQ ID NO:210 include those having GenBank accession numbers NM_{—}182925, AY233383, AY233382, X69878, U43143, and X68203.

Attenuating Expression of an mRNA:

The phrase, “attenuating expression of an mRNA,” as used herein, means administering or expressing an amount of a combination of interfering RNAs (e.g., siRNAs) to reduce translation of the target mRNAs into protein, either through mRNA cleavage or through direct inhibition of translation. The reduction in expression of the target mRNAs or the corresponding proteins is commonly referred to as “knock-down” and is reported relative to levels present following administration or expression of a non-targeting control RNA (e.g., non-targeting control siRNA). Knock-down of expression of an amount including and between 50% and 100% is contemplated by embodiments herein. However, it is not necessary that such knock-down levels be achieved for purposes of the present invention. In embodiments of the invention, an interfering RNA targeting KDR (VEGFR-2) mRNA and an interfering RNA targeting at least one of Tie-2, PDGFRA, PDGFRB, FLT1 (VEGFR-1), KIT, CSF1R, FLT3, FLT4 (VEGFR-3) variant 1 and FLT4 (VEGFR-3) variant 2 target mRNAs, are administered essentially in combination to reduce expression of these select mRNAs.

Knock-down is commonly assessed by measuring the mRNA levels using quantitative polymerase chain reaction (qPCR) amplification or by measuring protein levels by western blot or enzyme-linked immunosorbent assay (ELISA). Analyzing the protein level provides an assessment of both mRNA cleavage as well as translation inhibition. Further techniques for measuring knock-down include RNA solution hybridization, nuclease protection, northern hybridization, gene expression monitoring with a microarray, antibody binding, radioimmunoassay, and fluorescence activated cell analysis.

Inhibition of targets cited herein is also inferred in a human or mammal by observing an improvement in an ocular neovascularization-related condition symptom such as decreased production of new blood vessels, decreased leakage and edema, improvement in visual field loss or retinal microaneurysms, improvement in retinal edema, diabetic retinopathy, retinal ischemia, or in posterior segment neovascularization (PSNV), for example.

Interfering RNA:

In one embodiment of the invention, interfering RNA (e.g., siRNA) has a sense strand and an antisense strand, and the sense and antisense strands comprise a region of at least near-perfect contiguous complementarity of at least 19 nucleotides. In a further embodiment of the invention, the interfering RNA comprises a region of at least 13, 14, 15, 16, 17, or 18 contiguous nucleotides having percentages of sequence complementarity to or, having percentages of sequence identity with, the penultimate 13, 14, 15, 16, 17, or 18 nucleotides, respectively, of the 3′ end of the corresponding target sequence within an mRNA.

The length of each strand of the interfering RNA comprises 19 to 49 nucleotides, and may comprise a length of 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides.

The antisense strand of an siRNA is the active guiding agent of the siRNA in that the antisense strand is incorporated into RISC, thus allowing RISC to identify target mRNAs with at least partial complementarity to the antisense siRNA strand for cleavage or translational repression.

In embodiments of the present invention, interfering RNA target sequences (e.g., siRNA target sequences) within a target mRNA sequence are selected using available design tools. Interfering RNAs corresponding to the KDR (VEGFR-2) target sequence and at least one of the Tie-2, PDGFRA, PDGFRB, FLT1, KIT, CSF1R, FLT3, FLT4, FLT4 variant 1 and FLT4 variant 2 target sequences are then tested by transfection of cells expressing the target mRNAs followed by assessment of knockdown as described above. A combination of interfering RNAs that produces a knockdown in expression between 50% and 100% for each of the selected targets is selected for further analysis.

Techniques for selecting target sequences for siRNAs are provided by Tuschl, T. et al., “The siRNA User Guide,” revised May 6, 2004, available on the Rockefeller University web site; by Technical Bulletin #506, “siRNA Design Guidelines,” Ambion Inc. at Ambion's web site; and by other web-based design tools at, for example, the Invitrogen, Dharmacon, Integrated DNA Technologies, Genscript, or Proligo web sites. Initial search parameters can include G/C contents between 35% and 55% and siRNA lengths between 19 and 27 nucleotides. The target sequence may be located in the coding region or in the 5′ or 3′ untranslated regions of the mRNAs.

An embodiment of a 19-nucleotide DNA target sequence for KDR (VEGFR-2) receptor tyrosine kinase is present at nucleotides 922 to 940 of SEQ ID NO:1:

SEQ ID NO: 5
5′-GAAAGTTACCAGTCTATTA-3′.

An siRNA of the invention for targeting a corresponding mRNA sequence of SEQ ID NO:5 and having 21-nucleotide strands and a 2-nucleotide 3′ overhang is:

SEQ ID NO: 6
5′-GAAAGUUACCAGUCUAUUANN-3′
SEQ ID NO: 7
3′-NNCUUUCAAUGGUCAGAUAAU-5′.

Each “N” residue can be any nucleotide (A, C, G, U, T) or modified nucleotide. The 3′ end can have a number of “N” residues between and including 1, 2, 3, 4, 5, and 6. The “N” residues on either strand can be the same residue (e.g., UU, AA, CC, GG, or TT) or they can be different (e.g., AC, AG, AU, CA, CG, CU, GA, GC, GU, UA, UC, or UG). The 3′ overhangs can be the same or they can be different. In one embodiment, both strands have a 3′UU overhang.

An siRNA of the invention for targeting a corresponding mRNA sequence of SEQ ID NO:5 and having 21-nucleotide strands and a 3′UU overhang on each strand is:

SEQ ID NO: 8
5′-GAAAGUUACCAGUCUAUUAUU-3′
SEQ ID NO: 9
3′-UUCUUUCAAUGGUCAGAUAAU-5′.

The interfering RNA may also have a 5′ overhang of nucleotides or it may have blunt ends. An siRNA of the invention for targeting a corresponding mRNA sequence of SEQ ID NO:5 and having 19-nucleotide strands and blunt ends is:

SEQ ID NO: 10
5′-GAAAGUUACCAGUCUAUUA-′
SEQ ID NO: 11
3′-CUUUCAAUGGUCAGAUAAU-5′.

The strands of a double-stranded interfering RNA (e.g., an siRNA) may be connected to form a hairpin or stem-loop structure (e.g., an shRNA). An shRNA of the invention targeting a corresponding mRNA sequence of SEQ ID NO:8 and having a 19 bp double-stranded stem region and a 3′UU overhang is:

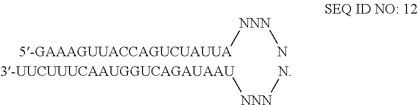

N is a nucleotide A, T, C, G, U, or a modified form known by one of ordinary skill in the art. The number of nucleotides N in the loop is a number between and including 3 to 23, or 5 to 15, or 7 to 13, or 4 to 9, or 9 to 11, or the number of nucleotides N is 9. Some of the nucleotides in the loop can be involved in base-pair interactions with other nucleotides in the loop. Examples of oligonucleotide sequences that can be used to form the loop include 5′-UUCAAGAGA-3′ (Brummelkamp, T. R. et al. (2002) Science 296: 550) and 5′-UUUGUGUAG-3′ (Castanotto, D. et al. (2002) RNA 8:1454). It will be recognized by one of skill in the art that the resulting single chain oligonucleotide forms a stem-loop or hairpin structure comprising a double-stranded region capable of interacting with the RNAi machinery.

The siRNA target sequence identified above can be extended at the 3′ end to facilitate the design of dicer-substrate 27-mer duplexes. Extension of the 19-nucleotide DNA target sequence (SEQ ID NO:5) identified in the KDR (VEGFR-2) receptor tyrosine kinase DNA sequence (SEQ ID NO:1) by 6 nucleotides yields a 25-nucleotide DNA target sequence present at nucleotides 922 to 946 of SEQ ID NO:1:

SEQ ID NO: 13
5′-GAAAGTTACCAGTCTATTATGTACA-3′.

A dicer-substrate 27-mer duplex of the invention for targeting a corresponding mRNA sequence of SEQ ID NO:13 is:

SEQ ID NO: 14
5′-GAAAGUUACCAGUCUAUUAUGUACA-3′
SEQ ID NO: 15
3′-UUCUUUCAAUGGUCAGAUAAUACAUGU-5′.

The two nucleotides at the 3′ end of the sense strand (i.e., the CA nucleotides of SEQ ID NO:14) may be deoxynucleotides for enhanced processing. Design of dicer-substrate 27-mer duplexes from 19-21 nucleotide target sequences, such as provided herein, is further discussed by the Integrated DNA Technologies (IDT) website and by Kim, D.-H. et al., (February, 2005) Nature Biotechnology 23:2; 222-226.

When interfering RNAs are produced by chemical synthesis, phosphorylation at the 5′ position of the nucleotide at the 5′ end of one or both strands (when present) can enhance siRNA efficacy and specificity of the bound RISC complex but is not required since phosphorylation can occur intracellularly.

Table 1 lists examples of KDR (VEGFR-2) receptor tyrosine kinase DNA target sequences of SEQ ID NO:1 from which siRNAs of the present invention are designed in a manner as set forth above. KDR encodes kinase insert domain-containing receptor; also known as VEGFR-2, as noted above.

TABLE 1
KDR (VEGFR-2) Target Sequences for siRNAs

| | # of Starting | |
|---|---|---|
| | Nucleotide with | SEQ |
| KDR (VEGFR-2) | reference to SEQ | ID |
| Target Sequence | ID NO: 1 | NO: |
| GAAAGTTACCAGTCTATTA | 922 | 16 |
| GTACATAGTTGTCGTTGTA | 942 | 17 |
| TCCGTCTCATGGAATTGAA | 990 | 18 |
| AGCAAGAACTGAACTAAAT | 1044 | 19 |
| TCAGCATAAGAAACTTGTA | 1104 | 20 |
| TGAGCACCTTAACTATAGA | 1169 | 21 |
| GGCATGTACTGACGATTAT | 1442 | 22 |
| ACTCAGGCATTGTATTGAA | 2432 | 23 |
| GGATGAACATTGTGAACGA | 2742 | 24 |
| GTGAACGACTGCCTTATGA | 2753 | 25 |
| CAAGATCCTCATTCATATT | 2961 | 26 |
| TTGGAAACCTGTCCACTTA | 3065 | 27 |
| TTCTTGGCATCGCGAAAGT | 3355 | 28 |
| ATATCCTCTTATCGGAGAA | 3401 | 29 |
| GGAGAAGAACGTGGTTAAA | 3414 | 30 |
| GGAAATCTCTTGCAAGCTA | 3784 | 31 |
| GAAATCTCTTGCAAGCTAA | 3785 | 32 |
| CTACATTGTTCTTCCGATA | 3825 | 33 |
| GTATGGTTCTTGCCTCAGA | 4085 | 34 |
| GATAGAGATTGGAGTGCAA | 4290 | 35 |
| GAGCTCTCCTCCTGTTTAA | 4356 | 36 |
| GCAGGAAGTAGCCGCATTT | 4453 | 37 |
| TTCATTTCGACAACAGAAA | 4476 | 38 |
| AGCCAGTCTTCTAGGCATA | 4515 | 39 |
| CTAGGCATATCCTGGAAGA | 4525 | 40 |
| AGATAAACCAGGCAACGTA | 4737 | 41 |
| TGATAGAAAGGAAGACTAA | 4863 | 42 |
| GAAAGGAAGACTAACGTTA | 4868 | 43 |
| AACGTTACCTTGCTTTGGA | 4880 | 44 |
| TGCTGTTTCTGACTCCTAA | 5042 | 45 |
| CTAATGAGAGTTCCTTCCA | 5057 | 46 |
| GAAAGGACATTCAGCTCAA | 5172 | 47 |
| GACATGCTATGGCACATAT | 5650 | 48 |
| GCATAACAAAGGTCATAAT | 5764 | 49 |
| TTGTAAACCGAGACCTAAA | 1118 | 164 |
| CAGTACGGCACCACTCAAA | 1609 | 165 |
| ATGTGAAGCGGTCAACAAA | 1890 | 166 |
| GTTCTCTAATAGCACAAAT | 2151 | 167 |
| GAGAATCAGACGACAAGTA | 2323 | 168 |
| GGCTACTTCTTGTCATCAT | 2639 | 169 |
| TCATCCTACGGACCGTTAA | 2654 | 170 |

Table 2 lists examples of Tie-2 DNA target sequences of SEQ ID NO:2 from which siRNAs of the present invention are designed in a manner as set forth above. Tie-2 encodes angiopoietin receptor, as noted above.

TABLE 2
TIE-2 Target Sequences for siRNAs

| | # of Starting | |
|---|---|---|
| | Nucleotide with | SEQ |
| TIE-2 | reference to SEQ | ID |
| Target Sequence | ID NO: 2 | NO: |
| AGATCAATGGTGCTTATTT | 432 | 50 |
| CTACCAGCTACTTTAACTA | 521 | 51 |
| GTACTCGGCCAGGTATATA | 712 | 52 |
| GCCGCTACCTACTAATGAA | 1273 | 53 |
| GCTACCTACTAATGAAGAA | 1276 | 54 |
| CCTTCAACATTTCTGTTAA | 1455 | 55 |
| CTGTTAAAGTTCTTCCAAA | 1467 | 56 |
| CCAAGAAGCTTCTATACAA | 1581 | 57 |
| CAAGAAGCTTCTATACAAA | 1582 | 58 |
| AAAGTCAGACCACTCTAAA | 1809 | 59 |
| TGACCTGGCAACCAATATT | 1830 | 60 |
| AGTGATCAGCAGAATATTA | 1904 | 61 |
| GTGATCAGCAGAATATTAA | 1905 | 62 |
| TTGACTTCGGTGCTACTTA | 1937 | 63 |
| TGACTTCGGTGCTACTTAA | 1938 | 64 |
| GGTGCTACTTAACAACTTA | 1945 | 65 |
| GTGATTTCTTGGACAATAT | 2114 | 66 |
| GGCTATTCTATTTCTTCTA | 2138 | 67 |
| TCTATTACTATCCGTTACA | 2153 | 68 |
| CTATTACTATCCGTTACAA | 2154 | 69 |
| GCACGTTGATGTGAAGATA | 2197 | 70 |
| ACGTTGATGTGAAGATAAA | 2199 | 71 |
| GGAATGACATCAAATTTCA | 2610 | 72 |
| ATGGACTACTTGAGCCAAA | 3002 | 73 |
| CCATCGAGTCACTGAATTA | 3165 | 74 |
| TGTATGATCTAATGAGACA | 3348 | 75 |
| AGATATTGGTGTCCTTAAA | 3408 | 76 |
| ATATTGGTGTCCTTAAACA | 3410 | 77 |
| CGAAAGACCTACGTGAATA | 3443 | 78 |
| GCCAAAGGATGTGATATAT | 3603 | 79 |
| GTGTACATATGTGCTGGAA | 3624 | 80 |
| GTACATATGTGCTGGAATT | 3626 | 81 |
| TGTGCTGGAATTCTAACAA | 3633 | 82 |
| CTAACAAGTCATAGGTTAA | 3645 | 83 |
| TCAGTCCAGGATGCTAACA | 3799 | 84 |
| CTGGTAATATTGACTTGTA | 3918 | 85 |
| GGAGACATGTGACATTTAT | 3974 | 86 |
| GTTGTGAGTTTACCTTGTA | 4051 | 87 |
| TGTGAGTTTACCTTGTATA | 4053 | 88 |
| AAATGTCTTGCCTACTCAA | 4110 | 89 |
| ACGTTTGGCAGAACTTGTA | 923 | 171 |
| GCCAGATCATATAGAAGTA | 1213 | 172 |
| AGAAGTAAACAGTGGTAAA | 1225 | 173 |
| GCTGGCCGCTACCTACTAA | 1269 | 174 |

Table 3 lists examples of PDGFRA and PDGFRB receptor tyrosine kinase DNA target sequences of SEQ ID NO:3 and SEQ ID NO:4, respectively, from which siRNAs of the present invention are designed in a manner as set forth above. PDGFRA encodes platelet-derived growth factor receptor alpha and PDGFRB encodes platelet-derived growth factor receptor beta, as noted above.

TABLE 3
PDGFRA and PDGFRB Target Sequences for siRNAs

| | # of Starting | |
|---|---|---|
| | Nucleotide with | SEQ |
| PDGFRA | reference to SEQ | ID |
| Target Sequence | ID NO: 3 | NO: |
| GCAGGCACATTTACATCTA | 489 | 90 |
| CTCTAGGAATGACGGATTA | 537 | 91 |
| TGATGATTCTGCCATTATA | 574 | 92 |
| CGAGACTCCTGTAACCTTA | 613 | 93 |
| GAAATAAGGTATCGAAGCA | 1262 | 94 |
| AAGGTATCGAAGCAAATTA | 1267 | 95 |
| AGGTATCGAAGCAAATTAA | 1268 | 96 |
| GGTATCGAAGCAAATTAAA | 1269 | 97 |
| GAAGACAGTGGCCATTATA | 1307 | 98 |
| TGGCCATTATACTATTGTA | 1315 | 99 |
| CACGCCGCTTCCTGATATT | 1465 | 100 |
| TGCGATGCCTGGCTAAGAA | 1644 | 101 |
| GGAACAGCCTATGGATTAA | 1976 | 102 |
| ACACGGAGCTATGTTATTT | 2297 | 103 |
| GGAGCGTTCTAAATATGAA | 3556 | 104 |
| CACTCAATCCATCCATGTA | 3842 | 105 |
| CCAACCTTGTTTAATAGAT | 4004 | 106 |
| CTACTACTGTTATCAGTAA | 4080 | 107 |
| AGTTGAGCATAGAGAACAA | 4257 | 108 |
| TTCTCAATGTAGAGGCATA | 4414 | 109 |
| CTCAATGTAGAGGCATAAA | 4416 | 110 |
| ATAAACCTGTGCTGAACAT | 4430 | 111 |
| TTGAAACTCGAGACCATAA | 4659 | 112 |
| TGAAACTCGAGACCATAAA | 4660 | 113 |
| GGAGGCTGGATGTGCATTA | 4692 | 114 |
| TTCAGGTTAGTGACATTTA | 4969 | 115 |
| CTAGCAATTGCGACCTTAA | 4999 | 116 |
| TAGCAATTGCGACCTTAAT | 5000 | 117 |
| CTGATAATTTGAGGTTAGA | 5259 | 118 |
| GATGAATTGTCACATCTAT | 5284 | 119 |
| TCTTTGCAATACTGCTTAA | 5341 | 120 |
| CTTAATTGCTGATACCATA | 5355 | 121 |
| GAAGATGCAGAAGCAATAA | 5433 | 122 |
| AGTTTCCAGTCCTAACAAA | 5750 | 123 |
| ATCACTGCCTTCGTTTATA | 6115 | 124 |
| ATTATACCTTGTCGCACAA | 587 | 175 |
| AGACTCCTGTAACCTTACA | 615 | 176 |
| GCATCACAATGCTGGAAGA | 918 | 177 |
| TCACAATGCTGGAAGAAAT | 921 | 178 |
| CAACCTGCATGAAGTCAAA | 1129 | 179 |
| GATATTGAGTGGATGATAT | 1478 | 180 |
| TGTCTGAACTGAAGATAAT | 2073 | 181 |
| GATCGTCCAGCCTCATATA | 2435 | 182 |
| ATCGTCCAGCCTCATATAA | 2436 | 183 |
| TCTACGAGATCATGGTGAA | 2922 | 184 |
| GGAACAGTGAGCCGGAGAA | 2946 | 185 |
| ATCATTCCTCTGCCTGACA | 3203 | 186 |
| TTGAAGACATCGACATGAT | 3348 | 187 |
| TGGACGACATCGGCATAGA | 3366 | 188 |
| CTTCAGACCTGGTGGAAGA | 3387 | 189 |
| | # of Starting | |
| | Nucleotide with | SEQ |
| PDGFRB | reference to SEQ | ID |
| Target Sequence | ID NO: 4 | NO: |
| GGAAACGGCTCTACATCTT | 807 | 125 |
| GAAACGGCTCTACATCTTT | 808 | 126 |
| GATGCCGAGGAACTATTCA | 860 | 127 |
| ATCTTTCTCACGGAAATAA | 878 | 128 |
| TCACGGAAATAACTGAGAT | 885 | 129 |
| ACCATTCCATGCCGAGTAA | 905 | 130 |
| TGGTGACACTGCACGAGAA | 939 | 131 |
| TGGATTCTGATGCCTACTA | 1065 | 132 |
| TCAACTTCGAGTGGACATA | 1197 | 133 |
| TGACGGAGAGTGTGAATGA | 1347 | 134 |
| CCTTCCAGCTACAGATCAA | 1692 | 135 |
| CGATGAAAGTGGCCGTCAA | 2352 | 136 |
| CAACGAGTCTCCAGTGCTA | 2845 | 137 |
| GGAACGTGCTCATCTGTGA | 2958 | 138 |
| TCAACCATCTCCTGTGACA | 3635 | 139 |
| TGGCTTAGGAGGCAAGAAA | 3954 | 140 |
| TACTGAGGTGGTAAATTAA | 4162 | 141 |
| CCATTAGGCAGCCTAATTA | 4391 | 142 |
| GAATAAGTCGGACTTATTA | 4742 | 143 |
| TGCCAGCACTAACATTCTA | 4774 | 144 |
| CACTAACATTCTAGAGTAT | 4780 | 145 |
| GATTCCAGATCACACATCA | 4882 | 146 |
| GGACAGTTATGTCTTGTAA | 5183 | 147 |
| GACAGTTATGTCTTGTAAA | 5184 | 148 |
| ATTGCAGGTTGGCACCTTA | 5478 | 149 |
| TGCAGGTTGGCACCTTACT | 5480 | 150 |
| GGTTCTCAATACGGTACCA | 5538 | 151 |
| TTCTCAATACGGTACCAAA | 5540 | 152 |
| CTCAATACGGTACCAAAGA | 5542 | 153 |
| TCAATACGGTACCAAAGAT | 5543 | 154 |
| CAATACGGTACCAAAGATA | 5544 | 155 |
| ATACGGTACCAAAGATATA | 5546 | 156 |
| TACGGTACCAAAGATATAA | 5547 | 157 |
| ACGGTACCAAAGATATAAT | 5548 | 158 |
| ATAATCACCTAGGTTTACA | 5562 | 159 |
| TAATCACCTAGGTTTACAA | 5563 | 160 |
| CACCTAGGTTTACAAATAT | 5567 | 161 |
| GGACTCACGTTAACTCACA | 5591 | 162 |
| TGGTGCTTCTCACTCACAA | 5697 | 163 |
| TGGAGACTAACGTGACGTA | 1896 | 190 |
| ACGGCCATGAGTACATCTA | 2193 | 191 |
| ATTCTCAGGCCACGATGAA | 2340 | 192 |
| GGCCGTCAAGATGCTTAAA | 2362 | 193 |
| GCCGTCAAGATGCTTAAAT | 2363 | 194 |
| TGGACTACCTGCACCGCAA | 2538 | 195 |
| GAAAGGAGACGTCAAATAT | 2740 | 196 |
| GACGTCAAATATGCAGACA | 2747 | 197 |
| CAGACATCGAGTCCTCCAA | 2760 | 198 |
| GCCGAGCAACTTTGATCAA | 2829 | 199 |
| CAAGAACTGCGTCCACAGA | 2926 | 200 |
| ACTCGAATTACATCTCCAA | 3030 | 201 |
| CTCGAATTACATCTCCAAA | 3031 | 202 |
| CCATGAACGAGCAGTTCTA | 3192 | 203 |
| ATGCCTCCGACGAGATCTA | 3252 | 204 |

Table 4 lists examples of FLT1 receptor tyrosine kinase DNA target sequences of SEQ ID NO:205 from which siRNAs of the present invention are designed in a manner as set forth above. FLT1 encodes VEGFR-1, as noted above.

TABLE 4
FLT1 (VEGFR-1) Target Sequences for siRNAs

| | # of Starting | |
|---|---|---|
| | Nucleotide with | SEQ |
| FLT1 (VEGFR-1) | reference to SEQ | ID |
| Target Sequence | ID NO: 205 | NO: |
| TGCCTGAAATGGTGAGTAA | 437 | 211 |
| AAAGGCTGAGCATAACTAA | 464 | 212 |
| CTAGCTGTACCTACTTCAA | 577 | 213 |
| GACCTTTCGTAGAGATGTA | 647 | 214 |
| CTTTATACTTGTCGTGTAA | 1171 | 215 |
| CCATCATTCAAATCTGTTA | 1198 | 216 |
| TAACACCTCAGTGCATATA | 1215 | 217 |
| CTTACCGGCTCTCTATGAA | 1304 | 218 |
| TTACCGGCTCTCTATGAAA | 1305 | 219 |
| CGGAAGTTGTATGGTTAAA | 1343 | 220 |
| ACTCGTGGCTACTCGTTAA | 1402 | 221 |
| CAATCTTGCTGAGCATAAA | 1460 | 222 |
| AAACCTCACTGCCACTCTA | 1497 | 223 |
| CTCAGCGCATGGCAATAAT | 1766 | 224 |
| TCAGCGCATGGCAATAATA | 1767 | 225 |
| ACAATGCACTACAGTATTA | 2044 | 226 |
| AGCATACCTCACTGTTCAA | 2478 | 227 |
| CTCTTCTGGCTCCTATTAA | 2560 | 228 |
| ACTGACTACCTATCAATTA | 2623 | 229 |
| CTGACTACCTATCAATTAT | 2624 | 230 |
| GCATCAGCATTTGGCATTA | 2779 | 231 |
| CATCAGCATTTGGCATTAA | 2780 | 232 |
| TGATGGTGATTGTTGAATA | 2963 | 233 |
| ATGGAAATCTCTCCAACTA | 2990 | 234 |
| CCAACTACCTCAAGAGCAA | 3002 | 235 |
| CGAATCTATCTTTGACAAA | 3453 | 236 |
| TGAGTACTCTACTCCTGAA | 3615 | 237 |
| GAGTACTCTACTCCTGAAA | 3616 | 238 |
| GCCATACTGACAGGAAATA | 3769 | 239 |
| ACTTGAGAGTAACCAGTAA | 4064 | 240 |
| CTTGAGAGTAACCAGTAAA | 4065 | 241 |
| ACAACTCGGTGGTCCTGTA | 4229 | 242 |
| TGAAGAACACTACTGCTAA | 4465 | 243 |
| TTACTCAGTGTTAGAGAAA | 4493 | 244 |
| GCAGGACCAGTTTGATTGA | 4565 | 245 |
| TCCTCTAGCAGGCCTAAGA | 4708 | 246 |
| CTAGTAAGATGCACTGAAA | 5097 | 247 |
| TGATGGCCTTACACTGAAA | 5147 | 248 |
| CCAAACCAATTCACCAACA | 5372 | 249 |
| AGCATTAGCTGGCGCATAT | 5484 | 250 |
| CATTAGCTGGCGCATATTA | 5486 | 251 |
| ATTAGCTGGCGCATATTAA | 5487 | 252 |
| GCGCATATTAAGCACTTTA | 5495 | 253 |
| CGCATATTAAGCACTTTAA | 5496 | 254 |
| GGACTCAGGATATTAGTTA | 5568 | 255 |
| GACTCAGGATATTAGTTAA | 5569 | 256 |
| CTAAGCTGGCTCTGTTTGA | 5726 | 257 |

Table 5 lists examples of KIT receptor tyrosine kinase DNA target sequences of SEQ ID NO:206 from which siRNAs of the present invention are designed in a manner as set forth above. KIT encodes the receptor for stem cell factor, as noted above.

TABLE 5
KIT Target Sequences for siRNAs

| | # of Starting | |
|---|---|---|
| | Nucleotide with | SEQ |
| | reference to SEQ | ID |
| KIT Target Sequence | ID NO: 206 | NO: |
| CGACGAGATTAGGCTGTTA | 174 | 258 |
| AAACACGGCTTAAGCAATT | 319 | 259 |
| CACAGTGACGTGCACAATA | 708 | 260 |
| ACAGTGACGTGCACAATAA | 709 | 261 |
| CAGTGACGTGCACAATAAA | 710 | 262 |
| AGACTAAACTACAGGAGAA | 776 | 263 |
| ACGGTGACTTCAATTATGA | 812 | 264 |
| CAGTTCAGCGAGAGTTAAT | 852 | 265 |
| AGCAGTGGATCTATATGAA | 1058 | 266 |
| AACAGAACCTTCACTGATA | 1075 | 267 |
| ACAGAACCTTCACTGATAA | 1076 | 268 |
| CAGAACCTTCACTGATAAA | 1077 | 269 |
| CTTCATCTAACGAGATTAA | 1150 | 270 |
| GTCCAATTCTGACGTCAAT | 1203 | 271 |
| CTAGTGGTTCAGAGTTCTA | 1435 | 272 |
| ATGGCACGGTTGAATGTAA | 1478 | 273 |
| CTGGCATGATGTGCATTAT | 1619 | 274 |
| TTGTGATGATTCTGACCTA | 1637 | 275 |
| TGATGATTCTGACCTACAA | 1640 | 276 |
| AGGTTGTTGAGGAGATAAA | 1694 | 277 |
| ACTTCCTTATGATCACAAA | 1746 | 278 |
| GCAACTGCTTATGGCTTAA | 1837 | 279 |
| AACTGCTTATGGCTTAATT | 1839 | 280 |
| CTCATGGTCGGATCACAAA | 2423 | 281 |
| CATGGTCGGATCACAAAGA | 2425 | 282 |
| TAAAGGAAACGCTCGACTA | 2496 | 283 |
| TGGAATGCCGGTCGATTCT | 2634 | 284 |
| CGGTCGATTCTAAGTTCTA | 2642 | 285 |
| TCGATTCTAAGTTCTACAA | 2645 | 286 |
| GACCATTCTGTGCGGATCA | 2875 | 287 |
| AAAGGTTCCAACTGTATAT | 3135 | 288 |
| AAGGTTCCAACTGTATATA | 3136 | 289 |
| GTTGATAGTTTACCTGAAT | 3278 | 290 |
| CCATAGTAGTATGATGATA | 3332 | 291 |
| CTAAGTCCTTTATGTGGAA | 3371 | 292 |
| TGAGACATAGGCCATGAAA | 3464 | 293 |
| ACTTGTATATACGCATCTA | 3616 | 294 |
| ACCATAAGGTTTCGTTTCT | 3670 | 295 |
| GTAGATTAAGAGCCATATA | 3726 | 296 |
| TGTAGATTCTGTGGAACAA | 3843 | 297 |
| CTTATGTAGCAGGAAATAA | 3979 | 298 |
| AGTAACTTGGCTTTCATTA | 4142 | 299 |
| CCATAGTGGTGCAGAGGAA | 4218 | 300 |
| TTCCTTAGACCTTCCATAA | 4319 | 301 |
| TCCTTAGACCTTCCATAAT | 4320 | 302 |
| GACTGTAGCCTGGATATTA | 4377 | 303 |
| TCAGGTATGTTGCCTTTAT | 4515 | 304 |
| AGAGAACTGTGGCCGTTAT | 4568 | 305 |
| TAAGCGGCGTAAGTTTAAA | 5000 | 306 |

Table 6 lists examples of CSF1R receptor tyrosine kinase DNA target sequences of SEQ ID NO:207 from which siRNAs of the present invention are designed in a manner as set forth above. CSF1R encodes Homo sapiens colony stimulating factor 1 receptor, as noted above.

TABLE 6
CSF1R Target Sequences for siRNAs

| | # of Starting | |
|---|---|---|
| | Nucleotide with | SEQ |
| CSF1R | reference to SEQ | ID |
| Target Sequence | ID NO: 207 | NO: |
| CCAGCAGCGTTGATGTTAA | 969 | 307 |
| CCTCAACCTCGATCAAGTA | 1078 | 308 |
| TCAACCTCGATCAAGTAGA | 1080 | 309 |
| CAACCTCGATCAAGTAGAT | 1081 | 310 |
| TCCAACATGCCGGCAACTA | 1101 | 311 |
| TAGAGAGTGCCTACTTGAA | 1179 | 312 |
| GGAGAGCTCTGACGTTTGA | 1458 | 313 |
| AGCGTCATATGGACATTCA | 1505 | 314 |
| TCATATGGACATTCATCAA | 1509 | 315 |
| GCTGACTGTTGAGACCTTA | 1705 | 316 |
| TCCTGCTGCTATTGTACAA | 1890 | 317 |
| TGCTGCTATTGTACAAGTA | 1893 | 318 |
| CTGCTATTGTACAAGTATA | 1895 | 319 |
| AGATCATCGAGAGCTATGA | 1944 | 320 |
| GCTATGGCGACCTGCTCAA | 2292 | 321 |
| CTGCTCAACTTTCTGCGAA | 2303 | 322 |
| GGAGGCGTCGACTATAAGA | 2375 | 323 |
| ATAAGAACATCCACCTCGA | 2388 | 324 |
| AGAACATCCACCTCGAGAA | 2391 | 325 |
| GCCTTCCTCGCTTCCAAGA | 2591 | 326 |
| GTAACGTGCTGTTGACCAA | 2637 | 327 |
| GAACAGCAAGTTCTATAAA | 2875 | 328 |
| AGTTCTATAAACTGGTGAA | 2883 | 329 |
| CTTCGGTCATTTCACTCAA | 3303 | 330 |
| TCGGTCATTTCACTCAACA | 3305 | 331 |
| CCTCGTGTTTGCTATGCCA | 3470 | 332 |
| TTTGCTATGCCAACTAGTA | 3477 | 333 |
| CAACTAGTAGAACCTTCTT | 3487 | 334 |
| GTAGAACCTTCTTTCCTAA | 3493 | 335 |
| AGAACCTTCTTTCCTAATC | 3495 | 336 |
| TGGAAATGGACTGACTTTA | 3526 | 337 |
| TGGACTGACTTTATGCCTA | 3532 | 338 |
| GGACTGACTTTATGCCTAT | 3533 | 339 |
| ACTGACTTTATGCCTATGA | 3535 | 340 |
| CTGACTTTATGCCTATGAA | 3536 | 341 |
| CAGGATGGCTCCTCTAAGA | 3688 | 342 |
| GGATGGCTCCTCTAAGAAT | 3690 | 343 |
| CATACTGGTACTGCTGTAA | 3776 | 344 |
| GAGCCAAGTGGCAGCTAAA | 3796 | 345 |
| AGCTGACTCATCCTAACTA | 3914 | 346 |
| GCTGACTCATCCTAACTAA | 3915 | 347 |

Table 7 lists examples of FLT3 receptor tyrosine kinase DNA target sequences of SEQ ID NO:208 from which siRNAs of the present invention are designed in a manner as set forth above. FLT3 encodes Homo sapiens FMS-related tyrosine kinase 3.

TABLE 7
FLT3 Target Sequences for siRNAs

| | # of Starting | |
|---|---|---|
| | Nucleotide with | SEQ |
| FLT3 | reference to SEQ | ID |
| Target Sequence | ID NO: 208 | NO: |
| AGAGTGAAGCTACCAATTA | 494 | 348 |
| AAAGTCCAGCTGTTGTTAA | 683 | 349 |
| AAGTCCAGCTGTTGTTAAA | 684 | 350 |
| CAGACCACATTGCCACAAT | 817 | 351 |
| GACCACATTGCCACAATTA | 819 | 352 |
| ACCACATTGCCACAATTAT | 820 | 353 |
| TGGTTACCATCGTAGGAAA | 1079 | 354 |
| CCAATTCAAGTGAAGATTA | 1115 | 355 |
| GATAACGGATACAGCATAT | 1249 | 356 |
| GTCAAGTGCTGTGCATACA | 1591 | 357 |
| TGCTAATTTGTCACAAGTA | 1736 | 358 |
| GTGACCGGCTCCTCAGATA | 1798 | 359 |
| TGACCGGCTCCTCAGATAA | 1799 | 360 |
| CTACGTTGATTTCAGAGAA | 1827 | 361 |
| TACGTTGATTTCAGAGAAT | 1828 | 362 |
| ACGTTGATTTCAGAGAATA | 1829 | 363 |
| CAATCCAGGTTGCCGTCAA | 1970 | 364 |
| AATCCAGGTTGCCGTCAAA | 1971 | 365 |
| TGATCTTCTCAACTATCTA | 2148 | 366 |
| CAAGAGAAGTTCAGATACA | 2276 | 367 |
| GGACTTGAATGTGCTTACA | 2391 | 368 |
| TGGATTGGCTCGAGATATC | 2547 | 369 |
| CATGAGTGATTCCAACTAT | 2565 | 370 |
| GAAGGCATCTACACCATTA | 2641 | 371 |
| CGGTTGATGCTAACTTCTA | 2735 | 372 |
| ATGCAGAAGAAGCGATGTA | 2903 | 373 |
| GTTTCGGAATGTCCTCACA | 2941 | 374 |
| GAAGATTCGTAGAGGAACA | 3028 | 375 |
| CCTAACAGGCTGTAGATTA | 3081 | 376 |
| CTAACAGGCTGTAGATTAC | 3082 | 377 |
| ACAGGCTGTAGATTACCAA | 3085 | 378 |
| TAGAAGCCGTCTGCGTTTA | 3169 | 379 |
| AGAAGCCGTCTGCGTTTAC | 3170 | 380 |
| GAAGCCGTCTGCGTTTACT | 3171 | 381 |
| TCTGCGTTTACTCTTGTTT | 3178 | 382 |
| CGGCTTGAGTGAATTGTGT | 3299 | 383 |
| GAATTGTGTACCTGAAGTA | 3309 | 384 |
| GTACCTGAAGTACAGTATA | 3316 | 385 |
| TACCTGAAGTACAGTATAT | 3317 | 386 |
| TGCTAAGGAGAAGCTAATA | 3365 | 387 |
| GGAGAAGCTAATATGATTT | 3371 | 388 |

Table 8 lists examples of FLT4 common target sequences, and FLT4 variant 1 and variant 2 receptor tyrosine kinase DNA target sequences of SEQ ID NO:209 and SEQ ID NO:210, respectively, from which siRNAs of the present invention are designed in a manner as set forth above. FLT4 encodes Homo sapiens FMS-related tyrosine kinase 4, as noted above.

TABLE 8
FLT4 Target Sequences in Common,
and FLT4 variant 1 and variant 2 Target
Sequences for siRNAs

| | # of Starting | |
|---|---|---|
| FLT4 variant 1 and | Nucleotide with | SEQ |
| variant 2 common | reference to SEQ | ID |
| Target Sequences | ID NO: 209 | NO: |
| GTGAGTGACTACTCCATGA | 84 | 389 |
| GCTACGTCTGCTACTACAA | 343 | 390 |
| TGTTCGTGAGAGACTTTGA | 409 | 391 |
| GACTTTGAGCAGCCATTCA | 420 | 392 |
| TTGAGCAGCCATTCATCAA | 424 | 393 |
| AGCAGCCATTCATCAACAA | 427 | 394 |
| TCACAGGCAACGAGCTCTA | 691 | 395 |
| CAGGCAACGAGCTCTATGA | 694 | 396 |
| ATGTGTGCAAGGCCAACAA | 943 | 397 |
| CAGGAGACGAGCTGGTGAA | 1060 | 398 |
| CCGAGTTCCAGTGGTACAA | 1111 | 399 |
| AGCATCTACTCGCGTCACA | 1317 | 400 |
| AGCAAGACCTCATGCCACA | 1456 | 401 |
| GCTTCACCATCGAATCCAA | 1690 | 402 |
| GCCATCCGAGGAGCTACTA | 1709 | 403 |
| TGCCAAGCCGACAGCTACA | 1752 | 404 |
| CGCTTCTGCTCGACTGCAA | 1834 | 405 |
| ACAAGCACTGCCACAAGAA | 2005 | 406 |
| TCGACTTGGCGGACTCCAA | 2191 | 407 |
| TGGAGATCGTGATCCTTGT | 2341 | 408 |
| CCGCTTTCGGCATCCACAA | 2608 | 409 |
| CGCTGATGTCGGAGCTCAA | 2695 | 410 |
| CTGATGTCGGAGCTCAAGA | 2697 | 411 |
| AAAGCGACGTGGTGAAGAT | 3160 | 412 |
| CTGAAAGCATCTTCGACAA | 3268 | 413 |
| TACGCCACATCATGCTGAA | 3451 | 414 |
| GATAGAGAGCAGGCATAGA | 3878 | 415 |
| GAGCAGGCATAGACAAGAA | 3884 | 416 |
| | # of Starting | |
| | Nucleotide with | SEQ |
| FLT4 variant 1 | reference to SEQ | ID |
| Target Sequence | ID NO: 209 | NO: |
| TGGTTGAACTCTGGTGGCA | 4199 | 417 |
| GTTGAACTCTGGTGGCACA | 4201 | 418 |
| ATGTCATTTAGTTCAGCAT | 4355 | 419 |
| CTTTGGCGACCTCCTTTCA | 4418 | 420 |
| TTTGGCGACCTCCTTTCAT | 4419 | 421 |
| TTGGCGACCTCCTTTCATC | 4420 | 422 |
| TGGCGACCTCCTTTCATCA | 4421 | 423 |
| TGTTGGAGGTTAAGGCATA | 4503 | 424 |
| TGGAGGTTAAGGCATACGA | 4506 | 425 |
| GTTAAGGCATACGAGAGCA | 4511 | 426 |
| CTGACCAAACAGCCAACTA | 4674 | 427 |
| TGACCAAACAGCCAACTAG | 4675 | 428 |
| ATTATACGCTGGCAACACA | 4722 | 429 |
| TATACGCTGGCAACACAGA | 4724 | 430 |
| | # of Starting | |
| | Nucleotide with | SEQ |
| FLT4 variant 2 | reference to SEQ | ID |
| Target Sequence | ID NO: 210 | NO: |
| GCTATTTCTTCTACTGCTA | 4002 | 431 |
| CTTCTACTGCTATCTACTA | 4009 | 432 |
| CTTATGCCAGCGTGACAGA | 4293 | 433 |
| GCTCACCTCTTGCCTTCTA | 4314 | 434 |
| CTAGGTCACTTCTCACAAT | 4330 | 435 |
| CGCCGATTATTCCTTGGTA | 4375 | 436 |
| GCCGATTATTCCTTGGTAA | 4376 | 437 |
| CCGATTATTCCTTGGTAAT | 4377 | 438 |
| TCCTTGGTAATATGAGTAA | 4385 | 439 |

As cited in the examples above, one of skill in the art is able to use the target sequence information provided in Tables 1-8 to design interfering RNAs having a length shorter or longer than the sequences provided in Table 1-8 by referring to the sequence position in SEQ ID NO:1, SEQ ID NO:2, SEQ ID NO:3, SEQ ID NO:4, SEQ ID NO: 205, SEQ ID NO:206, SEQ ID NO:207, SEQ ID NO:208, SEQ ID NO:209, or SEQ ID NO:210 and adding or deleting nucleotides complementary or near complementary to SEQ ID NO:1, SEQ ID NO:2, SEQ ID NO:3, SEQ ID NO:4, SEQ ID NO: 205, SEQ ID NO:206, SEQ ID NO:207, SEQ ID NO:208, SEQ ID NO:209, or SEQ ID NO:210, respectively.

The target RNA cleavage reaction guided by siRNAs and other forms of interfering RNA is highly sequence specific. In general, siRNA containing a sense nucleotide strand identical in sequence to a portion of the target mRNA and an antisense nucleotide strand exactly complementary to a portion of the target mRNA are siRNA embodiments for inhibition of mRNAs cited herein. However, 100% sequence complementarity between the antisense siRNA strand and the target mRNA, or between the antisense siRNA strand and the sense siRNA strand, is not required to practice the present invention. Thus, for example, the invention allows for sequence variations that might be expected due to genetic mutation, strain polymorphism, or evolutionary divergence.

In one embodiment of the invention, the antisense strand of the siRNA has at least near-perfect contiguous complementarity of at least 19 nucleotides with the target mRNA. “Near-perfect,” as used herein, means the antisense strand of the siRNA is “substantially complementary to,” and the sense strand of the siRNA is “substantially identical” to at least a portion of the target mRNA. “Identity,” as known by one of ordinary skill in the art, is the degree of sequence relatedness between nucleotide sequences as determined by matching the order and identity of nucleotides between the sequences. In one embodiment, the antisense strand of an siRNA having 80% and between 80% up to 100% complementarity, for example, 85%, 90% or 95% complementarity, to the target mRNA sequence are considered near-perfect complementarity and may be used in the present invention. “Perfect” contiguous complementarity is standard Watson-Crick base pairing of adjacent base pairs. “At least near-perfect” contiguous complementarity includes “perfect” complementarity as used herein. Computer methods for determining identity or complementarity are designed to identify the greatest degree of matching of nucleotide sequences, for example, BLASTN (Altschul, S. F., et al. (1990) J. Mol. Biol. 215:403-410).

The term “percent identity” describes the percentage of contiguous nucleotides in a first nucleic acid molecule that is the same as in a set of contiguous nucleotides of the same length in a second nucleic acid molecule. The term “percent complementarity” describes the percentage of contiguous nucleotides in a first nucleic acid molecule that can base pair in the Watson-Crick sense with a set of contiguous nucleotides in a second nucleic acid molecule.

The relationship between a target mRNA (sense strand) and one strand of an siRNA (the sense strand) is that of identity. The sense strand of an siRNA is also called a passenger strand, if present. The relationship between a target mRNA (sense strand) and the other strand of an siRNA (the antisense strand) is that of complementarity. The antisense strand of an siRNA is also called a guide strand.

In one embodiment of the invention, the region of contiguous nucleotides is a region of at least 14 contiguous nucleotides having at least 85% sequence complementarity to, or at least 85% sequence identity with, the penultimate 14 nucleotides of the 3′ end of an mRNA corresponding to the sequence identified by each sequence identifier. Two nucleotide substitutions (i.e., 12/14=86% identity/complementarity) are included in such a phrase.

In a further embodiment of the invention, the region of contiguous nucleotides is a region of at least 15, 16, 17, or 18 contiguous nucleotides having at least 80% sequence complementarity to, or at least 80% sequence identity with, the penultimate 14 nucleotides of the 3′ end of an mRNA corresponding to the sequence of the sequence identifier. Three nucleotide substitutions are included in such a phrase.

The target sequence in the mRNAs corresponding to SEQ ID NO:1, SEQ ID NO:2, SEQ ID NO:3, SEQ ID NO:4, SEQ ID NO: 205, SEQ ID NO:206, SEQ ID NO:207, SEQ ID NO:208, SEQ ID NO:209, or SEQ ID NO:210 may be in the 5′ or 3′ untranslated regions of the mRNA as well as in the coding region of the mRNA.

One or both of the strands of double-stranded interfering RNA may have a 3′ overhang of from 1 to 6 nucleotides, which may be ribonucleotides or deoxyribonucleotides or a mixture thereof. The nucleotides of the overhang are not base-paired. In one embodiment of the invention, the interfering RNA comprises a 3′ overhang of TT or UU. In another embodiment of the invention, the interfering RNA comprises at least one blunt end. The termini usually have a 5′ phosphate group or a 3′ hydroxyl group. In other embodiments, the antisense strand has a 5′ phosphate group, and the sense strand has a 5′ hydroxyl group. In still other embodiments, the termini are further modified by covalent addition of other molecules or functional groups.

The sense and antisense strands of the double-stranded siRNA may be in a duplex formation of two single strands as described above or may be a single molecule where the regions of complementarity are base-paired and are covalently linked by a hairpin loop so as to form a single strand. It is believed that the hairpin is cleaved intracellularly by a protein termed dicer to form an interfering RNA of two individual base-paired RNA molecules.

Interfering RNAs may differ from naturally-occurring RNA by the addition, deletion, substitution or modification of one or more nucleotides. Non-nucleotide material may be bound to the interfering RNA, either at the 5′ end, the 3′ end, or internally. Such modifications are commonly designed to increase the nuclease resistance of the interfering RNAs, to improve cellular uptake, to enhance cellular targeting, to assist in tracing the interfering RNA, to further improve stability, or to reduce the potential for activation of the interferon pathway. For example, interfering RNAs may comprise a purine nucleotide at the ends of overhangs. Conjugation of cholesterol to the 3′ end of the sense strand of an siRNA molecule by means of a pyrrolidine linker, for example, also provides stability to an siRNA.

Further modifications include a 3′ terminal biotin molecule, a peptide known to have cell-penetrating properties, a nanoparticle, a peptidomimetic, a fluorescent dye, or a dendrimer, for example.

Nucleotides may be modified on their base portion, on their sugar portion, or on the phosphate portion of the molecule and function in embodiments of the present invention. Modifications include substitutions with alkyl, alkoxy, amino, deaza, halo, hydroxyl, thiol groups, or a combination thereof, for example. Nucleotides may be substituted with analogs with greater stability such as replacing a ribonucleotide with a deoxyribonucleotide, or having sugar modifications such as 2′ OH groups replaced by 2′ amino groups, 2′ O-methyl groups, 2′ methoxyethyl groups, or a 2′-O, 4′-C methylene bridge, for example. Examples of a purine or pyrimidine analog of nucleotides include a xanthine, a hypoxanthine, an azapurine, a methylthioadenine, 7-deaza-adenosine and O- and N-modified nucleotides. The phosphate group of the nucleotide may be modified by substituting one or more of the oxygens of the phosphate group with nitrogen or with sulfur (phosphorothioates). Modifications are useful, for example, to enhance function, to improve stability or permeability, or to direct localization or targeting.

There may be a region or regions of the antisense interfering RNA strand that is (are) not complementary to a portion of SEQ ID NO:1, SEQ ID NO:2, SEQ ID NO:3, SEQ ID NO:4, SEQ ID NO: 205, SEQ ID NO:206, SEQ ID NO:207, SEQ ID NO:208, SEQ ID NO:209, or SEQ ID NO:210. Non-complementary regions may be at the 3′, 5′ or both ends of a complementary region or between two complementary regions.

Interfering RNAs may be generated exogenously by chemical synthesis, by in vitro transcription, or by cleavage of longer double-stranded RNA with dicer or another appropriate nuclease with similar activity. Chemically synthesized interfering RNAs, produced from protected ribonucleoside phosphoramidites using a conventional DNA/RNA synthesizer, may be obtained from commercial suppliers such as Ambion Inc. (Austin, Tex.), Invitrogen (Carlsbad, Calif.), or Dharmacon (Lafayette, Colo.). Interfering RNAs are purified by extraction with a solvent or resin, precipitation, electrophoresis, chromatography, or a combination thereof, for example. Alternatively, interfering RNA may be used with little if any purification to avoid losses due to sample processing.

Interfering RNAs can also be expressed endogenously from plasmid or viral expression vectors or from minimal expression cassettes, for example, PCR generated fragments comprising one or more promoters and an appropriate template or templates for the interfering RNA. Examples of commercially available plasmid-based expression vectors for shRNA include members of the pSilencer series (Ambion, Austin, Tex.) and pCpG-siRNA (InvivoGen, San Diego, Calif.). Viral vectors for expression of interfering RNA may be derived from a variety of viruses including adenovirus, adeno-associated virus, lentivirus (e.g., HIV, FIV, and EIAV), and herpes virus. Examples of commercially available viral vectors for shRNA expression include pSilencer adeno (Ambion, Austin, Tex.) and pLenti6/BLOCK-iT™-DEST (Invitrogen, Carlsbad, Calif.). Selection of viral vectors, methods for expressing the interfering RNA from the vector and methods of delivering the viral vector are within the ordinary skill of one in the art. Examples of kits for production of PCR-generated shRNA expression cassettes include Silencer Express (Ambion, Austin, Tex.) and siXpress (Mirus, Madison, Wis.). A first interfering RNA may be administered via in vivo expression from a first expression vector capable of expressing the first interfering RNA and a second interfering RNA may be administered via in vivo expression from a second expression vector capable of expressing the second interfering RNA, or both interfering RNAs may be administered via in vivo expression from a single expression vector capable of expressing both interfering RNAs.

Interfering RNAs may be expressed from a variety of eukaryotic promoters known to those of ordinary skill in the art, including pol III promoters, such as the U6 or H1 promoters, or pol II promoters, such as the cytomegalovirus promoter. Those of skill in the art will recognize that these promoters can also be adapted to allow inducible expression of the interfering RNA.

Hybridization under Physiological Conditions:

In certain embodiments of the present invention, an antisense strand of an interfering RNA hybridizes with an mRNA in vivo as part of the RISC complex.

For example, high stringency conditions could occur at about 50% formamide at 37° C. to 42° C. Reduced stringency conditions could occur at about 35% to 25% formamide at 30° C. to 35° C. Examples of stringency conditions for hybridization are provided in Sambrook, J., 1989, Molecular Cloning: A Laboratory Manual, Cold Spring Harbor Laboratory Press, Cold Spring Harbor, N.Y. Further examples of stringent hybridization conditions include 400 mM NaCl, 40 mM PIPES pH 6.4, 1 mM EDTA, 50° C. or 70° C. for 12-16 hours followed by washing, or hybridization at 70° C. in 1×SSC or 50° C. in 1×SSC, 50% formamide followed by washing at 70° C. in 0.3×SSC, or hybridization at 70° C. in 4×SSC or 50° C. in 4×SSC, 50% formamide followed by washing at 67° C. in 1×SSC. The temperature for hybridization is about 5-10° C. less than the melting temperature (Tₘ) of the hybrid where Tₘ is determined for hybrids between 19 and 49 base pairs in length using the following calculation: Tₘ° C.=81.5+16.6(log₁₀[Na+])+0.41 (% G+C)−(600/N) where N is the number of bases in the hybrid, and [Na+] is the concentration of sodium ions in the hybridization buffer.

The above-described in vitro hybridization assay provides a method of predicting whether binding between a candidate siRNA and a target will have specificity. However, in the context of the RISC complex, specific cleavage of a target can also occur with an antisense strand that does not demonstrate high stringency for hybridization in vitro.

Single-Stranded Interfering RNA:

As cited above, interfering RNAs ultimately function as single strands. Single-stranded (ss) interfering RNA has been found to effect mRNA silencing, albeit less efficiently than double-stranded RNA. Therefore, embodiments of the present invention also provide for administration of a ss interfering RNA that hybridizes under physiological conditions to a portion of SEQ ID NO:1, SEQ ID NO:2, SEQ ID NO:3, SEQ ID NO:4, SEQ ID NO: 205, SEQ ID NO:206, SEQ ID NO:207, SEQ ID NO:208, SEQ ID NO:209, or SEQ ID NO:210 and has a region of at least near-perfect contiguous complementarity of at least 19 nucleotides with the hybridizing portion of SEQ ID NO:1, SEQ ID NO:2, SEQ ID NO:3, SEQ ID NO:4, SEQ ID NO: 205, SEQ ID NO:206, SEQ ID NO:207, SEQ ID NO:208, SEQ ID NO:209, or SEQ ID NO:210, respectively. The ss interfering RNA has a length of 19 to 49 nucleotides as for the ds interfering RNA cited above. The ss interfering RNA has a 5′ phosphate or is phosphorylated in situ or in vivo at the 5′ position. The term “5′ phosphorylated” is used to describe, for example, polynucleotides or oligonucleotides having a phosphate group attached via ester linkage to the C5 hydroxyl of the sugar (e.g., ribose, deoxyribose, or an analog of same) at the 5′ end of the polynucleotide or oligonucleotide.

SS interfering RNAs are synthesized chemically or by in vitro transcription or expressed endogenously from vectors or expression cassettes as for ds interfering RNAs. 5′ Phosphate groups may be added via a kinase, or a 5′ phosphate may be the result of nuclease cleavage of an RNA. Delivery is as for ds interfering RNAs. In one embodiment, ss interfering RNAs having protected ends and nuclease resistant modifications are administered for silencing. SS interfering RNAs may be dried for storage or dissolved in an aqueous solution. The solution may contain buffers or salts to inhibit annealing or for stabilization.

Hairpin Interfering RNA:

A hairpin interfering RNA is a single molecule (e.g., a single oligonucleotide chain) that comprises both the sense and antisense strands of an interfering RNA in a stem-loop or hairpin structure (e.g., a shRNA). For example, shRNAs can be expressed from DNA vectors in which the DNA oligonucleotides encoding a sense interfering RNA strand are linked to the DNA oligonucleotides encoding the reverse complementary antisense interfering RNA strand by a short spacer. If needed for the chosen expression vector, 3′ terminal T's and nucleotides forming restriction sites may be added. The resulting RNA transcript folds back onto itself to form a stem-loop structure.

Mode of Administration:

Interfering RNA may be delivered directly to the eye by ocular tissue administration such as periocular, conjunctival, subtenon, intracameral, intravitreal, intraocular, subretinal, subconjunctival, retrobulbar, intracanalicular, or suprachoroidal administration; by injection, by direct application to the eye using a catheter or other placement device such as a retinal pellet, intraocular insert, suppository or an implant comprising a porous, non-porous, or gelatinous material; by topical ocular drops or ointments; or by a slow release device in the cul-de-sac or implanted adjacent to the sclera (transscleral) or within the eye. Intracameral injection may be through the cornea into the anterior chamber to allow the agent to reach the trabecular meshwork. Intracanalicular injection may be into the venous collector channels draining Schlemm's canal or into Schlemm's canal. Systemic or parenteral administration is contemplated including but not limited to intravenous, subcutaneous, transdermal, and oral delivery.

Administration of the combination of interfering RNAs as provided herein is such that they act together and such that the neovasularization targets of the combination are attenuated simultaneously. Simultaneous attenuation may be achieved by simultaneous administration of the combination of individual interfering RNAs or by sequential administration at time intervals such that the target mRNAs of the combination are attenuated in overlapping intervals of time. When the combination of interfering RNAs is delivered simultaneously, the interfering RNAs can be separate molecules or they can be linked to each other by covalent bonds (e.g., phosphodiester or disulfide bonds) or by non-covalent bonds.

Subject:

A subject, in an embodiment, in need of treatment for an ocular neovascularization-related condition or at risk for developing an ocular neovascularization-related condition is a human or other mammal having an ocular neovascularization-related condition or at risk of having an ocular neovascularization-related condition associated with undesired or inappropriate expression or activity of targets as cited herein, i.e., KDR (VEGFR-2) together with one or more of Tie-2, PDGFRA, PDGFRB, FLT1, KIT, CSF1R, FLT3, FLT4, FLT4 variant 1 or FLT4 variant 2. Ocular structures associated with such disorders may include the eye, retina, choroid, lens, cornea, trabecular meshwork, iris, optic nerve, optic nerve head, sclera, aqueous chamber, vitreous chamber, ciliary body, or posterior segment, for example. A subject may also be an ocular cell, cell culture, organ or an ex vivo organ or tissue.

Formulations and Dosage:

Pharmaceutical formulations comprise interfering RNAs, or salts thereof, of the invention up to 99% by weight mixed with a physiologically acceptable ophthalmic carrier medium such as water, buffer, saline, glycine, hyaluronic acid, mannitol, and the like.

Interfering RNAs of the present invention are administered as solutions, suspensions, or emulsions. The following are examples of possible formulations embodied by this invention.

| | Amount in weight % |
|---|---|
| Interfering RNA | up to 99; 0.1-99; 0.1-50; |
| | 0.5-10.0 |
| Hydroxypropylmethylcellulose | 0.5 |
| Sodium chloride | 0.8 |
| Benzalkonium Chloride | 0.01 |
| EDTA | 0.01 |
| NaOH/HCl | qs pH 7.4 |
| Purified water (RNase-free) | qs 100 Ml |
| Interfering RNA | up to 99; 0.1-99; 0.1-50; 0.5-10.0 |
| Phosphate Buffered Saline | 1.0 |
| Benzalkonium Chloride | 0.01 |
| Polysorbate 80 | 0.5 |
| Purified water (RNase-free) | q.s. to 100% |
| Interfering RNA | up to 99; 0.1-99; 0.1-50; 0.5-10.0 |
| Monobasic sodium phosphate | 0.05 |
| Dibasic sodium phosphate | 0.15 |
| (anhydrous) | |
| Sodium chloride | 0.75 |
| Disodium EDTA | 0.05 |
| Cremophor EL | 0.1 |
| Benzalkonium chloride | 0.01 |
| HCl and/or NaOH | pH 7.3-7.4 |
| Purified water (RNase-free) | q.s. to 100% |
| Interfering RNA | up to 99; 0.1-99; 0.1-50; 0.5-10.0 |
| Phosphate Buffered Saline | 1.0 |
| Hydroxypropyl-β-cyclodextrin | 4.0 |
| Purified water (RNase-free) | q.s. to 100% |

In general, the doses of combination compositions as provided herein will vary, but will be in an effective amount, which refers to an amount of a combination of interfering RNAs acting together during an overlapping interval of time, that effectively inhibits or causes regression of neovascularization or angiogenesis, thereby preventing or treating retinal edema, AMD, DR, sequela associated with retinal ischemia, or PSNV, for example, in a human patient.

Generally, an effective amount of the interfering RNAs of embodiments of the invention results in an extracellular concentration at the surface of the target cell of from 100 pM to 1000 nM, or from 1 nM to 400 nM, or from 5 nM to about 100 nM, or about 10 nM. The dose required to achieve this local concentration will vary depending on a number of factors including the delivery method, the site of delivery, the number of cell layers between the delivery site and the target cell or tissue, whether delivery is local or systemic, etc. The concentration at the delivery site may be considerably higher than it is at the surface of the target cell or tissue. Topical compositions are delivered to the surface of the eye one to four times per day, or on an extended delivery schedule such as daily, weekly, bi-weekly, monthly, or longer, according to the routine discretion of a skilled clinician. The pH of the formulation is about pH 4-9, or pH 4.5 to pH 7.4.

Therapeutic treatment of patients with siRNAs directed against the ocular neovascularization-related condition target mRNAs is expected to be beneficial over small molecule topical ocular drops by increasing the duration of action, thereby allowing less frequent dosing and greater patient compliance.

While the precise regimen is left to the discretion of the clinician and/or health care provider, interfering RNAs may be administered by placing one drop in each eye as directed by the clinician. An effective amount of a formulation may depend on factors such as the age, race, and sex of the subject, the severity of the ocular hypertension, the rate of target gene transcript/protein turnover, the interfering RNA potency, and the interfering RNA stability, for example. In one embodiment, the interfering RNA is delivered topically to the eye and reaches the ocular vasculature of, for example, the retina at a therapeutic dose thereby ameliorating an ocular neovascularization-related condition-associated disease process.

Acceptable Carriers:

An ophthalmically acceptable carrier refers to those carriers that cause at most, little to no ocular irritation, provide suitable preservation if needed, and deliver one or more interfering RNAs of the present invention in a homogenous dosage. An acceptable carrier for administration of interfering RNA of embodiments of the present invention include the cationic lipid-based transfection reagents TransIT®-TKO (Mims Corporation, Madison, Wis.), LIPOFECTIN®, Lipofectamine, OLIGOFECTAMINE™ (Invitrogen, Carlsbad, Calif.), or DHARMAFECT™ (Dharmacon, Lafayette, Colo.); polycations such as polyethyleneimine; cationic peptides such as Tat, polyarginine, or Penetratin (Antp peptide); or liposomes. Liposomes are formed from standard vesicle-forming lipids and a sterol, such as cholesterol, and may include a targeting molecule such as a monoclonal antibody having binding affinity for endothelial cell surface antigens, for example. Further, the liposomes may be PEGylated liposomes.

The interfering RNAs may be delivered in solution, in suspension, or in bioerodible or non-bioerodible delivery devices. The interfering RNAs can be delivered alone or as components of defined, covalent conjugates. The interfering RNAs can also be complexed with cationic lipids, cationic peptides, or cationic polymers; complexed with proteins, fusion proteins, or protein domains with nucleic acid binding properties (e.g., protamine); or encapsulated in nanoparticles. Tissue- or cell-specific delivery can be accomplished by the inclusion of an appropriate targeting moiety such as an antibody or antibody fragment.

For ophthalmic delivery, an interfering RNA may be combined with ophthalmologically acceptable preservatives, co-solvents, surfactants, viscosity enhancers, penetration enhancers, buffers, sodium chloride, or water to form an aqueous, sterile ophthalmic suspension or solution. Ophthalmic solution formulations may be prepared by dissolving the interfering RNA in a physiologically acceptable isotonic aqueous buffer. Further, the ophthalmic solution may include an ophthalmologically acceptable surfactant to assist in dissolving the inhibitor. Viscosity building agents, such as hydroxymethyl cellulose, hydroxyethyl cellulose, methylcellulose, polyvinylpyrrolidone, or the like may be added to the compositions of the present invention to improve the retention of the compound.

In order to prepare a sterile ophthalmic ointment formulation, the interfering RNA is combined with a preservative in an appropriate vehicle, such as mineral oil, liquid lanolin, or white petrolatum. Sterile ophthalmic gel formulations may be prepared by suspending the interfering RNA in a hydrophilic base prepared from the combination of, for example, CARBOPOL®-940 (BF Goodrich, Charlotte, N.C.), or the like, according to methods known in the art for other ophthalmic formulations. VISCOAT® (Alcon Laboratories, Inc., Fort Worth, Tex.) may be used for intraocular injection, for example. Other compositions of the present invention may contain penetration enhancing agents such as cremephor and TWEEN® 80 (polyoxyethylene sorbitan monolaureate, Sigma Aldrich, St. Louis, Mo.), in the event the interfering RNA is less penetrating in the eye.

Kits:

Embodiments of the present invention provide a kit that includes reagents for attenuating the expression of an mRNA as cited herein in a cell. The kit contains an siRNA or an shRNA expression vector. For siRNAs and non-viral shRNA expression vectors the kit also may contain a transfection reagent or other suitable delivery vehicle. For viral shRNA expression vectors, the kit may contain the viral vector and/or the necessary components for viral vector production (e.g., a packaging cell line as well as a vector comprising the viral vector template and additional helper vectors for packaging). The kit may also contain positive and negative control siRNAs or shRNA expression vectors (e.g., a non-targeting control siRNA or an siRNA that targets an unrelated mRNA). The kit also may contain reagents for assessing knockdown of the intended target gene (e.g., primers and probes for quantitative PCR to detect the target mRNA and/or antibodies against the corresponding protein for western blots). Alternatively, the kit may comprise an siRNA sequence or an shRNA sequence and the instructions and materials necessary to generate the siRNA by in vitro transcription or to construct an shRNA expression vector.

A pharmaceutical combination in kit form is further provided that includes, in packaged combination, a carrier means adapted to receive a container means in close confinement therewith and a first container means including an interfering RNA composition and an ophthalmically acceptable carrier. Such kits can further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, can also be included in the kit.

The ability of interfering RNA to knock-down the levels of endogenous target gene expression in, for example, human umbilical vein endothelial cells (HUVEC cells) is evaluated in vitro as follows. HUVEC cells (ATCC CRL-1730), are plated 24-48 h prior to transfection in MCDB-131 Complete Medium (VEC Technologies, Rensselaer, N.Y.). Transfection is performed using Dharmafect 1 (Dharmacon, Lafayette, Colo.) according to the manufacturer's instructions at interfering RNA (e.g., siRNA) concentrations ranging from 0.1 nM-100 nM. siCONTROL™ Non-Targeting siRNA #1 and siCONTROL™ Cyclophilin B siRNA (Dharmacon) are used as negative and positive controls, respectively. Target mRNA levels and cyclophilin B mRNA (PPIB, NM_{—}000942) levels are assessed by qPCR 24 h post-transfection using, for example, TAQMAN® forward and reverse primers and a probe set that preferably encompasses the target site (Applied Biosystems, Foster City, Calif.). The positive control siRNA gives essentially complete knockdown of cyclophilin B mRNA when transfection efficiency is 100%. Therefore, target mRNA knockdown is corrected for transfection efficiency by reference to the cyclophilin B mRNA level in HUVEC cells transfected with the cyclophilin B siRNA. Target protein levels may be assessed approximately 72 h post-transfection (actual time dependent on protein turnover rate) by western blot, for example. Standard techniques for RNA and/or protein isolation from cultured cells are well-known to those skilled in the art. To reduce the chance of non-specific, off-target effects, the lowest possible concentration of interfering RNA is used that produces the desired level of knock-down in target gene expression.

The references cited herein, to the extent that they provide exemplary procedural or other details supplementary to those set forth herein, are specifically incorporated by reference.

Those of skill in the art, in light of the present disclosure, will appreciate that obvious modifications of the embodiments disclosed herein can be made without departing from the spirit and scope of the invention. All of the embodiments disclosed herein can be made and executed without undue experimentation in light of the present disclosure. The full scope of the invention is set out in the disclosure and equivalent embodiments thereof. The specification should not be construed to unduly narrow the full scope of protection to which the present invention is entitled.

### EXAMPLES

### Example 1

### Interfering RNA for Specifically Silencing KDR (VEGFR2) in bEnd.3 Cells

The present study examines the ability of KDR-interfering RNA to knock down the levels of endogenous KDR protein expression in cultured bEnd.3 cells.

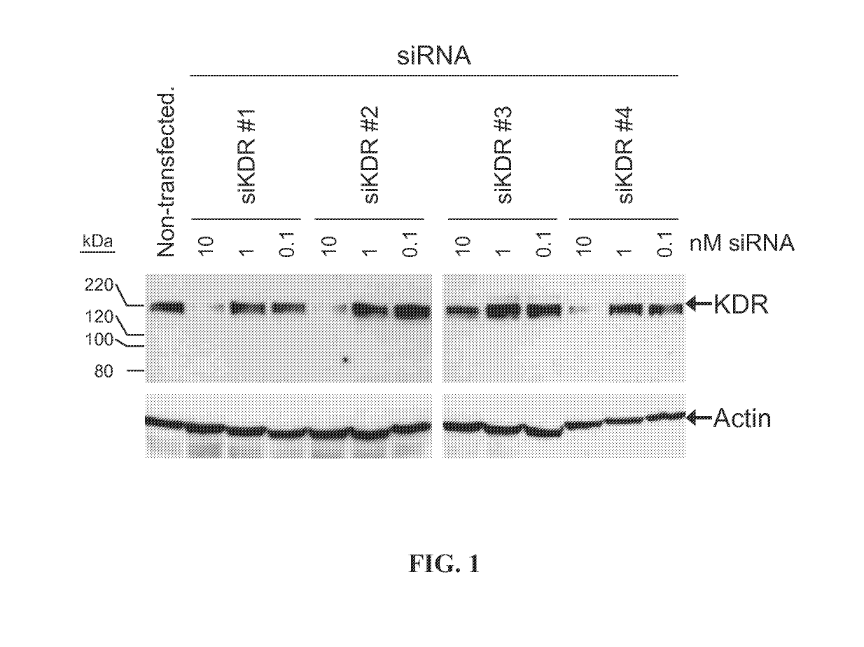

The The murine cell line bEnd.3 (ATCC, Manassas, Va.; Montesano et al. Cell 62:435-445, 1990) was transfected using standard in vitro concentrations (0.1-10 nM) of KDR siRNAs and DHARMAFECT® #1 transfection reagent (Dharmacon, Lafayette, Colo.). All siRNAs were dissolved in 1× siRNA buffer, an aqueous solution of 20 mM KCl, 6 mM HEPES (pH 7.5), 0.2 mM MgCl₂. Western blots using an anti-KDR antibody (Cell Signaling, Danvers, Mass.) were performed to assess KDR protein expression at 72 h post-transfection. KDR siRNAs (Dharmacon, Lafayette, Colo.) are double-stranded interfering RNAs having specificity for murine KDR(NM_{—}010612). SiKDR #1 targets the sequence GGAGACACGUGGAGGAUUU (SEQ ID NO: 440); siKDR #2 targets the sequence GAUGAAACCUAUCAGUCUA (SEQ ID NO: 441); siKDR #3 targets the sequence GAUAUCAAAUGGUACAGAA (SEQ ID NO: 442); and sKDR #4 targets the sequence CGACAUAGCCUCCACUGUU (SEQ ID NO: 443). As shown by the data of FIG. 1, siKDR #1, siKDR #2, and siKDR #4 reduced KDR protein expression effectively at 10 nM, but not at lower concentrations, relative to non-transfected cells, indicating that these KDR siRNAs are more effective than siKDR #3.

### Example 2

### Interfering RNA for Specifically Silencing TIE2 (TEK) in bEnd.3 Cells

The present study examines the ability of TIE2-interfering RNA to knock down the levels of endogenous TIE2 protein expression in cultured bEnd.3 cells.

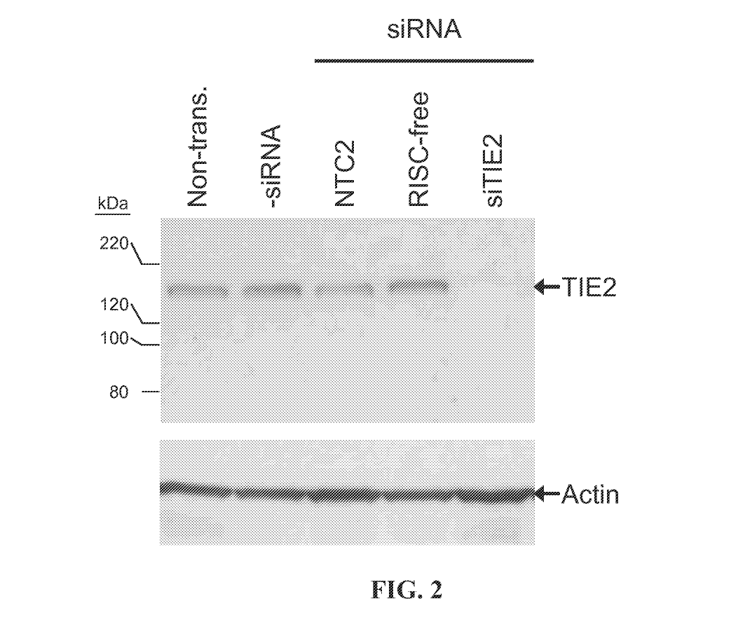

Transfection of bEnd.3 cells was accomplished using standard in vitro concentrations (100 nM) of TIE2 siRNA, siCONTROL Non-targeting siRNA #2 (NTC2), or siCONTROL RISC-free siRNA #1 and DHARMAFECT® #1 transfection reagent (Dharmacon, Lafayette, Colo.). All siRNAs were dissolved in 1× siRNA buffer, an aqueous solution of 20 mM KCl, 6 mM HEPES (pH 7.5), 0.2 mM MgCl₂. Control samples included a buffer control in which the volume of siRNA was replaced with an equal volume of 1× siRNA buffer (-siRNA) and non-transfected cells. Western blots using an anti-TIE2 antibody (BD Biosciences, San Jose, Calif.) were performed to assess TIE2 protein expression at 72 h post-transfection. The TIE2 siRNA (Dharmacon, Lafayette, Colo.) is a double-stranded interfering RNA having specificity for murine TIE2 (NM_{—}013690). SiTIE2 targets the sequence CCAAATGACTTCAACTATA (SEQ ID NO: 444). As shown by the data of FIG. 2, siTIE2 silenced TIE2 protein expression dramatically relative to the control siRNAs.

### Example 3

### Interfering RNA for Specifically Silencing KDR and TIE2 in bEnd.3 Cells

The present study examines the ability of mixtures of KDR- and TIE2-interfering RNAs to knock down simultaneously the levels of endogenous KDR and TIE2 protein expression in cultured bEnd.3 cells.

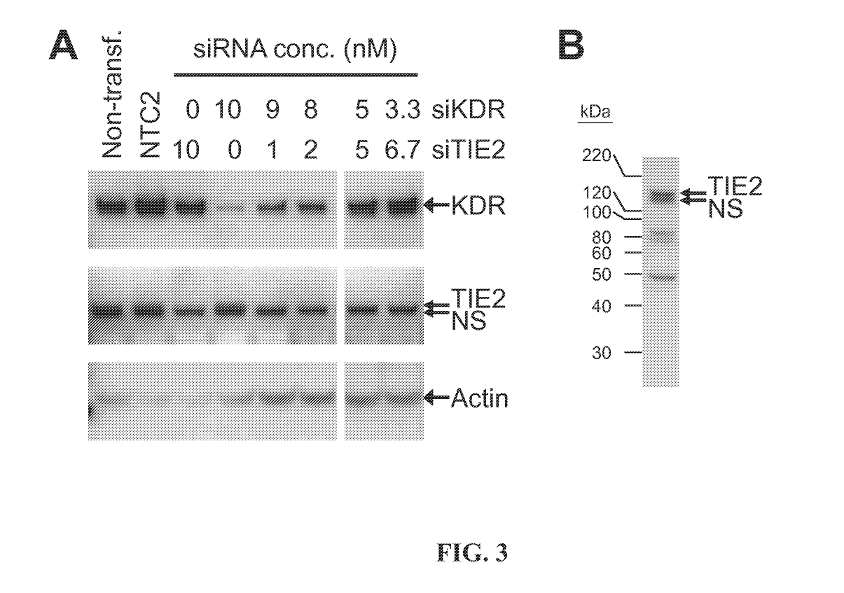

Transfection of bEnd.3 cells was accomplished using standard in vitro concentrations (1-10 nM) of KDR siRNA and TIE2 siRNA, or siCONTROL Non-targeting siRNA #2 (NTC2, 10 nM) and DHARMAFECT® #1 transfection reagent (Dharmacon, Lafayette, Colo.). All siRNAs were dissolved in 1× siRNA buffer, an aqueous solution of 20 mM KCl, 6 mM HEPES (pH 7.5), 0.2 mM MgCl₂. Western blots using an anti-KDR antibody (Cell Signaling, Danvers, Mass.) and an anti-TIE2 antibody (Santa Cruz Biotechnology, Santa Cruz, Calif.) were performed to assess KDR and TIE2 protein expression at 72 h post-transfection. The KDR and TIE2 siRNAs (Dharmacon, Lafayette, Colo.) are double-stranded interfering RNAs having specificity for murine KDR (NM_{—}010612) and TIE2 (NM_{—}013690), respectively. SiKDR targets the sequence GGAGACACGUGGAGGAUUU (SEQ ID NO: 440), and siTIE2 targets the sequence CCAAATGACTTCAACTATA (SEQ ID NO: 444). As shown by the data of FIG. 3A (upper panel), 10 nM siKDR silences expression of KDR protein, but not of TIE2 protein. Similarly, 10 nM siTIE2 silences expression of TIE2 protein, but not of KDR protein (middle panel). Unlike the TIE2 antibody used in Example 2, which recognizes a single band at ˜140 kDa, the TIE2 antibody used in this example also recognizes an additional, non-specific band at slightly less than 140 kDa, as shown in FIG. 3B. Careful inspection of the middle panel of FIG. 3A reveals that the expression of the upper, TIE2-specific band is silenced at all siTIE2 concentrations from 1-10 nM. Expression of the lower, non-specific band is not affected by the siTIE2 siRNA. Most importantly, mixtures of 8 nM siKDR and 2 nM siTIE2 and of 9 nM siKDR and 1 nM siTIE2 silenced expression of both KDR and TIE2 proteins relatively effectively. Further adjustment of the relative siRNA concentrations will allow more efficient silencing of both targets.

What is claimed is:

1. A method of treating an ocular neovascularization-related condition in a subject in need thereof, comprising:
administering to an eye of said subject a composition comprising an effective amount of a first and a second interfering RNA and a pharmaceutically acceptable carrier,
wherein each interfering RNA has a length of 19 to 49 nucleotides and comprises a sense nucleotide strand, an antisense nucleotide strand, and a region of at least near-perfect contiguous complementarity of at least 19 nucleotides; and
wherein said antisense strand of said first interfering RNA hybridizes under physiological conditions to a portion of mRNA corresponding to SEQ ID NO:1, and has a region of at least near-perfect contiguous complementarity of at least 19 nucleotides with said hybridizing portion of mRNA corresponding to SEQ ID NO:1, and
wherein said antisense strand of said second interfering RNA hybridizes under physiological conditions to a portion of mRNA corresponding to SEQ ID NO:2, SEQ ID NO:3, SEQ ID NO:4, SEQ ID NO:205, SEQ ID NO:206, SEQ ID NO:207, SEQ ID NO:208, SEQ ID NO:209, or SEQ ID NO:210, and has a region of at least near-perfect contiguous complementarity of at least 19 nucleotides with said hybridizing portion of mRNA corresponding to SEQ ID NO:2, SEQ ID NO:3, SEQ ID NO:4, SEQ ID NO:205, SEQ ID NO:206, SEQ ID NO:207, SEQ ID NO:208, SEQ ID NO:209, or SEQ ID NO:210, respectively;
wherein said ocular neovascularization-related condition is treated thereby.

2. The method of claim 1, wherein said subject is a human.

3. The method of claim 1, wherein said antisense strand of said second interfering RNA hybridizes under physiological conditions to a portion of mRNA corresponding to SEQ ID NO:2 and has a region of at least near-perfect contiguous complementarity of at least 19 nucleotides with said hybridizing portion of mRNA corresponding to SEQ ID NO:2.

4. The method of claim 1, wherein said antisense strand of said second interfering RNA hybridizes under physiological conditions to a portion of mRNA corresponding to SEQ ID NO:3 or SEQ ID NO:4 and has a region of at least near-perfect contiguous complementarity of at least 19 nucleotides with said hybridizing portion of mRNA corresponding to SEQ ID NO:3 or SEQ ID NO:4, respectively.

5. The method of claim 1, wherein said antisense strand of said second interfering RNA hybridizes under physiological conditions to a portion of mRNA corresponding to SEQ ID NO:205, SEQ ID NO:206, SEQ ID NO:207, SEQ ID NO:208, SEQ ID NO:209, or SEQ ID NO:210, and has a region of at least near-perfect contiguous complementarity of at least 19 nucleotides with said hybridizing portion of mRNA corresponding to SEQ ID NO:205, SEQ ID NO:206, SEQ ID NO:207, SEQ ID NO:208, SEQ ID NO:209, or SEQ ID NO:210, respectively.

6. The method of claim 1, wherein said antisense strand of said first interfering RNA is designed to target an mRNA corresponding to SEQ ID NO:1 comprising nucleotide 922, 942, 990, 1044, 1104, 1169, 1442, 2432, 2742, 2753, 2961, 3065, 3355, 3401, 3414, 3784, 3785, 3825, 4085, 4290, 4356, 4453, 4476, 4515, 4525, 4737, 4863, 4868, 4880, 5042, 5057, 5172, 5650, 5764, 1118, 1609, 1890, 2151, 2323, 2639, or 2654.

7. The method of claim 1, wherein said antisense strand of said second interfering RNA is designed to target an mRNA corresponding to SEQ ID NO:2 comprising nucleotide 432, 521, 712, 1273, 1276, 1455, 1467, 1581, 1582, 1809, 1830, 1904, 1905, 1937, 1938, 1945, 2114, 2138, 2153, 2154, 2197, 2199, 2610, 3002, 3165, 3348, 3408, 3410, 3443, 3603, 3624, 3626, 3633, 3645, 3799, 3918, 3974, 4051, 4053, 4110, 923, 1213, 1225, or 1269.

8. The method of claim 1, wherein said antisense strand of said second interfering RNA is designed to target an mRNA corresponding to SEQ ID NO:3 comprising nucleotide 489, 537, 574, 613, 1262, 1267, 1268, 1269, 1307, 1315, 1465, 1644, 1976, 2297, 3556, 3842, 4004, 4080, 4257, 4414, 4416, 4430, 4659, 4660, 4692, 4969, 4999, 5000, 5259, 5284, 5341, 5355, 5433, 5750, 6115, 587, 615, 918, 921, 1129, 1478, 2073, 2435, 2436, 2922, 2946, 3203, 3348, 3366, or 3387; or corresponding to SEQ ID NO:4 comprising nucleotide 807, 808, 860, 878, 885, 905, 939, 1065, 1197, 1347, 1692, 2352, 2845, 2958, 3635, 3954, 4162, 4391, 4742, 4774, 4780, 4882, 5183, 5184, 5478, 5480, 5538, 5540, 5542, 5543, 5544, 5546, 5547, 5548, 5562, 5563, 5567, 5591, 5697, 1896, 2193, 2340, 2362, 2363, 2538, 2740, 2747, 2760, 2829, 2926, 3030, 3031, 3192, or 3252.

9. The method of claim 1, wherein said antisense strand of said second interfering RNA is designed to target an mRNA corresponding to SEQ ID NO:205 comprising nucleotide 437, 464, 577, 647, 1171, 1198, 1215, 1304, 1305, 1343, 1402, 1460, 1497, 1766, 1767, 2044, 2478, 2560, 2623, 2624, 2779, 2780, 2963, 2990, 3002, 3453, 3615, 3616, 3769, 4064, 4065, 4229, 4465, 4493, 4565, 4708, 5097, 5147, 5372, 5484, 5486, 5487, 5495, 5496, 5568, 5569, or 5726.

10. The method of claim 1, further comprising administering to said subject a third interfering RNA having a length of 19 to 49 nucleotides and comprising
a sense nucleotide strand, an antisense nucleotide strand, and a region of at least near-perfect complementarity of at least 19 nucleotides;
wherein said antisense strand of said third interfering RNA hybridizes under physiological conditions to a portion of mRNA corresponding to SEQ ID NO:3 or SEQ ID NO:4, and said antisense strand has a region of at least near-perfect contiguous complementarity of at least 19 nucleotides with said hybridizing portion of mRNA corresponding to SEQ ID NO:3 or SEQ ID NO:4, respectively.

11. The method of claim 1, further comprising administering to said subject a third interfering RNA having a length of 19 to 49 nucleotides and comprising
a sense nucleotide strand, an antisense nucleotide strand, and a region of at least near-perfect complementarity of at least 19 nucleotides;
wherein said antisense strand of said third interfering RNA hybridizes under physiological conditions to a portion of mRNA corresponding to SEQ ID NO:205, SEQ ID NO:206, SEQ ID NO:207, SEQ ID NO:208, SEQ ID NO:209, or SEQ ID NO:210, and said antisense strand has a region of at least near-perfect contiguous complementarity of at least 19 nucleotides with said hybridizing portion of mRNA corresponding to SEQ ID NO:205, SEQ ID NO:206, SEQ ID NO:207, SEQ ID NO:208, SEQ ID NO:209, or SEQ ID NO:210, respectively.

12. The method of claim 1, further comprising administering to said subject a third interfering RNA having a length of 19 to 49 nucleotides and comprising:
a sense nucleotide strand, an antisense nucleotide strand, and a region of at least near-perfect complementarity of at least 19 nucleotides;
wherein said antisense strand of said third interfering RNA hybridizes under physiological conditions to a portion of mRNA corresponding to SEQ ID NO:205, SEQ ID NO:206, SEQ ID NO:207, SEQ ID NO:208, SEQ ID NO:209, or SEQ ID NO:210, and said antisense strand has a region of at least near-perfect contiguous complementarity of at least 19 nucleotides with said hybridizing portion of mRNA corresponding to SEQ ID NO:205, SEQ ID NO:206, SEQ ID NO:207, SEQ ID NO:208, SEQ ID NO:209, or SEQ ID NO:210, respectively.

13. The method of claim 1, wherein said sense nucleotide strand and said antisense nucleotide strand are connected by a loop nucleotide sequence.

14. The method of claim 1, wherein said composition is administered via a topical, intravitreal, transcleral, periocular, conjunctival, subtenon, intracameral, subretinal, subconjunctival, retrobulbar, intracanalicular or suprachoroidal route.

15. The method of claim 1, wherein said first interfering RNA is administered via in vivo expression from a first expression vector capable of expressing said first interfering RNA and said second interfering RNA is administered via in vivo expression from a second expression vector capable of expressing said second interfering RNA.

16. The method of claim 1, wherein said first interfering RNA is administered via in vivo expression from a first expression vector capable of expressing said first interfering RNA and said second interfering RNA is administered via in vivo expression from a second expression vector capable of expressing said second interfering RNA.

17. A composition comprising a first and a second interfering RNA, each interfering RNA having a length of 19 to 49 nucleotides, wherein said first interfering RNA comprises a nucleotide sequence of any one of SEQ ID NO:16-SEQ ID NO:49 and SEQ ID NO:164-SEQ ID NO:170, or a complement thereof; and said second interfering RNA comprises a nucleotide sequence of any one of SEQ ID NO:50-SEQ ID NO:89, SEQ ID NO:90-SEQ ID NO:124, SEQ ID NO:125-SEQ ID NO:163, SEQ ID NO:171-SEQ ID NO:174, SEQ ID NO:175-SEQ ID NO:189, SEQ ID NO:190-SEQ ID NO:204, and SEQ ID NO:211-SEQ ID NO:439, or a complement thereof, and a pharmaceutically acceptable carrier.

18. The composition of claim 17, wherein said interfering RNA is an shRNA, an siRNA, or an miRNA.

19. The method of claim 1, wherein said human has retinal edema, diabetic retinopathy, sequela associated with retinal ischemia, or posterior segment neovascularization.

20. A method of attenuating expression of an ocular neovascularization-related condition target mRNA first variant without attenuating expression of an ocular neovascularization-related condition target mRNA second variant in a subject, comprising:
administering to said subject a composition comprising an effective amount of interfering RNA having a length of 19 to 49 nucleotides and a pharmaceutically acceptable carrier, said interfering RNA comprising:
a region of at least 13 contiguous nucleotides having at least 90% sequence complementarity to, or at least 90% sequence identity with, said penultimate 13 nucleotides of said 3′ end of said first variant,
wherein said expression of said first variant mRNA is attenuated without attenuating expression of said second variant mRNA, and wherein said first variant target mRNA is SEQ ID NO:209, and said second variant target mRNA is SEQ ID NO:210.

21. A composition comprising an interfering RNA having a length of 19 to 49 nucleotides, wherein said interfering RNA comprises a nucleotide sequence that targets any one of SEQ ID NO:16-SEQ ID NO:49, SEQ ID NO:164-SEQ ID NO:170, SEQ ID NO:50-SEQ ID NO:89, SEQ ID NO:90-SEQ ID NO:124, SEQ ID NO:125-SEQ ID NO:163, SEQ ID NO:171-SEQ ID NO:174, SEQ ID NO:175-SEQ ID NO:189, SEQ ID NO:190-SEQ ID NO:204, and SEQ ID NO:211-SEQ ID NO:439, or a complement thereof, and a pharmaceutically acceptable carrier.


SEQUENCE LISTING (444 sequences)

SEQ ID NO: 1 - DNA, 5830 bp, Homo sapiens
actgagtcccgggaccccgggagagcggtcagtgtgtggtcgctgcgtttcctctgcctgcgccgggcatcacttgcgcgccgcagaaagtccgtctggcagcctggatatcctctcctaccggcacccgcagacgcccctgcagccgccggtcggcgcccgggctccctagccctgtgcgctcaactgtcctgcgctgcggggtgccgcgagttccacctccgcgcctccttctctagacaggcgctgggagaaagaaccggctcccgagttctgggcatttcgcccggctcgaggtgcaggatgcagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaaatcattattctagtaggcacggcggtgattgccatgttcttctggctacttcttgtcatcatcctacggaccgttaagcgggccaatggaggggaactgaagacaggctacttgtccatcgtcatggatccagatgaactcccattggatgaacattgtgaacgactgccttatgatgccagcaaatgggaattccccagagaccggctgaagctaggtaagcctcttggccgtggtgcctttggccaagtgattgaagcagatgcctttggaattgacaagacagcaacttgcaggacagtagcagtcaaaatgttgaaagaaggagcaacacacagtgagcatcgagctctcatgtctgaactcaagatcctcattcatattggtcaccatctcaatgtggtcaaccttctaggtgcctgtaccaagccaggagggccactcatggtgattgtggaattctgcaaatttggaaacctgtccacttacctgaggagcaagagaaatgaatttgtcccctacaagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaagctcctgaagatctgtataaggacttcctgaccttggagcatctcatctgttacagcttccaagtggctaagggcatggagttcttggcatcgcgaaagtgtatccacagggacctggcggcacgaaatatcctcttatcggagaagaacgtggttaaaatctgtgactttggcttggcccgggatatttataaagatccagattatgtcagaaaaggagatgctcgcctccctttgaaatggatggccccagaaacaatttttgacagagtgtacacaatccagagtgacgtctggtcttttggtgttttgctgtgggaaatattttccttaggtgcttctccatatcctggggtaaagattgatgaagaattttgtaggcgattgaaagaaggaactagaatgagggcccctgattatactacaccagaaatgtaccagaccatgctggactgctggcacggggagcccagtcagagacccacgttttcagagttggtggaacatttgggaaatctcttgcaagctaatgctcagcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcagtcagtatctgcagaacagtaagcgaaagagccggcctgtgagtgtaaaaacatttgaagatatcccgttagaagaaccagaagtaaaagtaatcccagatgacaaccagacggacagtggtatggttcttgcctcagaagagctgaaaactttggaagacagaaccaaattatctccatcttttggtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgactcggggaccacactgagctctcctcctgtttaaaaggaagcatccacaccccaactcccggacatcacatgagaggtctgctcagattttgaagtgttgttctttccaccagcaggaagtagccgcatttgattttcatttcgacaacagaaaaaggacctcggactgcagggagccagtcttctaggcatatcctggaagaggcttgtgacccaagaatgtgtctgtgtcttctcccagtgttgacctgatcctcttttttcattcatttaaaaagcattatcatgcccctgctgcgggtctcaccatgggtttagaacaaagagcttcaagcaatggccccatcctcaaagaagtagcagtacctggggagctgacacttctgtaaaactagaagataaaccaggcaacgtaagtgttcgaggtgttgaagatgggaaggatttgcagggctgagtctatccaagaggctttgtttaggacgtgggtcccaagccaagccttaagtgtggaattcggattgatagaaaggaagactaacgttaccttgctttggagagtactggagcctgcaaatgcattgtgtttgctctggtggaggtgggcatggggtctgttctgaaatgtaaagggttcagacggggtttctggttttagaaggttgcgtgttcttcgagttgggctaaagtagagttcgttgtgctgtttctgactcctaatgagagttccttccagaccgttagctgtctccttgccaagccccaggaagaaaatgatgcagctctggctccttgtctcccaggctgatcctttattcagaataccacaaagaaaggacattcagctcaaggctccctgccgtgttgaagagttctgactgcacaaaccagcttctggtttcttctggaatgaataccctcatatctgtcctgatgtgatatgtctgagactgaatgcgggaggttcaatgtgaagctgtgtgtggtgtcaaagtttcaggaaggattttacccttttgttcttccccctgtccccaacccactctcaccccgcaacccatcagtattttagttatttggcctctactccagtaaacctgattgggtttgttcactctctgaatgattattagccagacttcaaaattattttatagcccaaattataacatctattgtattatttagacttttaacatatagagctatttctactgatttttgcccttgttctgtcctttttttcaaaaaagaaaatgtgttttttgtttggtaccatagtgtgaaatgctgggaacaatgactataagacatgctatggcacatatatttatagtctgtttatgtagaaacaaatgtaatatattaaagccttatatataatgaactttgtactattcacattttgtatcagtattatgtagcataacaaaggtcataatgctttcagcaattgatgtcattttattaaagaacattgaaaaacttga

SEQ ID NO: 2 - DNA, 4138 bp, Homo sapiens
cttctgtgctgttccttcttgcctctaacttgtaaacaagacgtactaggacgatgctaatggaaagtcacaaaccgctgggtttttgaaaggatccttgggacctcatgcacatttgtggaaactggatggagagatttggggaagcatggactctttagccagcttagttctctgtggagtcagcttgctcctttctggaactgtggaaggtgccatggacttgatcttgatcaattccctacctcttgtatctgatgctgaaacatctctcacctgcattgcctctgggtggcgcccccatgagcccatcaccataggaagggactttgaagccttaatgaaccagcaccaggatccgctggaagttactcaagatgtgaccagagaatgggctaaaaaagttgtttggaagagagaaaaggctagtaagatcaatggtgcttatttctgtgaagggcgagttcgaggagaggcaatcaggatacgaaccatgaagatgcgtcaacaagcttccttcctaccagctactttaactatgactgtggacaagggagataacgtgaacatatctttcaaaaaggtattgattaaagaagaagatgcagtgatttacaaaaatggttccttcatccattcagtgccccggcatgaagtacctgatattctagaagtacacctgcctcatgctcagccccaggatgctggagtgtactcggccaggtatataggaggaaacctcttcacctcggccttcaccaggctgatagtccggagatgtgaagcccagaagtggggacctgaatgcaaccatctctgtactgcttgtatgaacaatggtgtctgccatgaagatactggagaatgcatttgccctcctgggtttatgggaaggacgtgtgagaaggcttgtgaactgcacacgtttggcagaacttgtaaagaaaggtgcagtggacaagagggatgcaagtcttatgtgttctgtctccctgacccctatgggtgttcctgtgccacaggctggaagggtctgcagtgcaatgaagcatgccaccctggtttttacgggccagattgtaagcttaggtgcagctgcaacaatggggagatgtgtgatcgcttccaaggatgtctctgctctccaggatggcaggggctccagtgtgagagagaaggcataccgaggatgaccccaaagatagtggatttgccagatcatatagaagtaaacagtggtaaatttaatcccatttgcaaagcttctggctggccgctacctactaatgaagaaatgaccctggtgaagccggatgggacagtgctccatccaaaagactttaaccatacggatcatttctcagtagccatattcaccatccaccggatcctcccccctgactcaggagtttgggtctgcagtgtgaacacagtggctgggatggtggaaaagcccttcaacatttctgttaaagttcttccaaagcccctgaatgccccaaacgtgattgacactggacataactttgctgtcatcaacatcagctctgagccttactttggggatggaccaatcaaatccaagaagcttctatacaaacccgttaatcactatgaggcttggcaacatattcaagtgacaaatgagattgttacactcaactatttggaacctcggacagaatatgaactctgtgtgcaactggtccgtcgtggagagggtggggaagggcatcctggacctgtgagacgcttcacaacagcttctatcggactccctcctccaagaggtctaaatctcctgcctaaaagtcagaccactctaaatttgacctggcaaccaatatttccaagctcggaagatgacttttatgttgaagtggagagaaggtctgtgcaaaaaagtgatcagcagaatattaaagttccaggcaacttgacttcggtgctacttaacaacttacatcccagggagcagtacgtggtccgagctagagtcaacaccaaggcccagggggaatggagtgaagatctcactgcttggacccttagtgacattcttcctcctcaaccagaaaacatcaagatttccaacattacacactcctcggctgtgatttcttggacaatattggatggctattctatttcttctattactatccgttacaaggttcaaggcaagaatgaagaccagcacgttgatgtgaagataaagaatgccaccatcattcagtatcagctcaagggcctagagcctgaaacagcataccaggtggacatttttgcagagaacaacatagggtcaagcaacccagccttttctcatgaactggtgaccctcccagaatctcaagcaccagcggacctcggaggggggaagatgctgcttatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttccttcgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtacgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctaggacagaacatctgtataccctctgtttccctttcactggcatgggagacccttgacaactgctgagaaaacatgcctctgccaaaggatgtgatatataagtgtacatatgtgctggaattctaacaagtcataggttaatatttaagacactgaaaaatctaagtgatataaatcagattcttctctctcattttatccctcacctgtagcatgccagtcccgtttcatttagtcatgtgaccactctgtcttgtgtttccacagcctgcaagttcagtccaggatgctaacatctaaaaatagacttaaatctcattgcttacaagcctaagaatctttagagaagtatacataagtttaggataaaataatgggattttcttttcttttctctggtaatattgacttgtatattttaagaaataacagaaagcctgggtgacatttgggagacatgtgacatttatatattgaattaatatccctacatgtattgcacattgtaaaaagttttagttttgatgagttgtgagtttaccttgtatactgtaggcacactttgcactgatatatcatgagtgaataaatgtcttgcctactcaaaaaaaaaaaa

SEQ ID NO: 3 - DNA, 6405 bp, Homo sapiens
ggtttttgagcccattactgttggagctacagggagagaaacagaggaggagactgcaagagatcattggaggccgtgggcacgctctttactccatgtgtgggacattcattgcggaataacatcggaggagaagtttcccagagctatggggacttcccatccggcgttcctggtcttaggctgtcttctcacagggctgagcctaatcctctgccagctttcattaccctctatccttccaaatgaaaatgaaaaggttgtgcagctgaattcatccttttctctgagatgctttggggagagtgaagtgagctggcagtaccccatgtctgaagaagagagctccgatgtggaaatcagaaatgaagaaaacaacagcggcctttttgtgacggtcttggaagtgagcagtgcctcggcggcccacacagggttgtacacttgctattacaaccacactcagacagaagagaatgagcttgaaggcaggcacatttacatctatgtgccagacccagatgtagcctttgtacctctaggaatgacggattatttagtcatcgtggaggatgatgattctgccattataccttgtcgcacaactgatcccgagactcctgtaaccttacacaacagtgagggggtggtacctgcctcctacgacagcagacagggctttaatgggaccttcactgtagggccctatatctgtgaggccaccgtcaaaggaaagaagttccagaccatcccatttaatgtttatgctttaaaagcaacatcagagctggatctagaaatggaagctcttaaaaccgtgtataagtcaggggaaacgattgtggtcacctgtgctgtttttaacaatgaggtggttgaccttcaatggacttaccctggagaagtgaaaggcaaaggcatcacaatgctggaagaaatcaaagtcccatccatcaaattggtgtacactttgacggtccccgaggccacggtgaaagacagtggagattacgaatgtgctgcccgccaggctaccagggaggtcaaagaaatgaagaaagtcactatttctgtccatgagaaaggtttcattgaaatcaaacccaccttcagccagttggaagctgtcaacctgcatgaagtcaaacattttgttgtagaggtgcgggcctacccacctcccaggatatcctggctgaaaaacaatctgactctgattgaaaatctcactgagatcaccactgatgtggaaaagattcaggaaataaggtatcgaagcaaattaaagctgatccgtgctaaggaagaagacagtggccattatactattgtagctcaaaatgaagatgctgtgaagagctatacttttgaactgttaactcaagttccttcatccattctggacttggtcgatgatcaccatggctcaactgggggacagacggtgaggtgcacagctgaaggcacgccgcttcctgatattgagtggatgatatgcaaagatattaagaaatgtaataatgaaacttcctggactattttggccaacaatgtctcaaacatcatcacggagatccactcccgagacaggagtaccgtggagggccgtgtgactttcgccaaagtggaggagaccatcgccgtgcgatgcctggctaagaatctccttggagctgagaaccgagagctgaagctggtggctcccaccctgcgttctgaactcacggtggctgctgcagtcctggtgctgttggtgattgtgatcatctcacttattgtcctggttgtcatttggaaacagaaaccgaggtatgaaattcgctggagggtcattgaatcaatcagcccagatggacatgaatatatttatgtggacccgatgcagctgccttatgactcaagatgggagtttccaagagatggactagtgcttggtcgggtcttggggtctggagcgtttgggaaggtggttgaaggaacagcctatggattaagccggtcccaacctgtcatgaaagttgcagtgaagatgctaaaacccacggccagatccagtgaaaaacaagctctcatgtctgaactgaagataatgactcacctggggccacatttgaacattgtaaacttgctgggagcctgcaccaagtcaggccccatttacatcatcacagagtattgcttctatggagatttggtcaactatttgcataagaatagggatagcttcctgagccaccacccagagaagccaaagaaagagctggatatctttggattgaaccctgctgatgaaagcacacggagctatgttattttatcttttgaaaacaatggtgactacatggacatgaagcaggctgatactacacagtatgtccccatgctagaaaggaaagaggtttctaaatattccgacatccagagatcactctatgatcgtccagcctcatataagaagaaatctatgttagactcagaagtcaaaaacctcctttcagatgataactcagaaggccttactttattggatttgttgagcttcacctatcaagttgcccgaggaatggagtttttggcttcaaaaaattgtgtccaccgtgatctggctgctcgcaacgtcctcctggcacaaggaaaaattgtgaagatctgtgactttggcctggccagagacatcatgcatgattcgaactatgtgtcgaaaggcagtacctttctgcccgtgaagtggatggctcctgagagcatctttgacaacctctacaccacactgagtgatgtctggtcttatggcattctgctctgggagatcttttcccttggtggcaccccttaccccggcatgatggtggattctactttctacaataagatcaagagtgggtaccggatggccaagcctgaccacgctaccagtgaagtctacgagatcatggtgaaatgctggaacagtgagccggagaagagaccctccttttaccacctgagtgagattgtggagaatctgctgcctggacaatataaaaagagttatgaaaaaattcacctggacttcctgaagagtgaccatcctgctgtggcacgcatgcgtgtggactcagacaatgcatacattggtgtcacctacaaaaacgaggaagacaagctgaaggactgggagggtggtctggatgagcagagactgagcgctgacagtggctacatcattcctctgcctgacattgaccctgtccctgaggaggaggacctgggcaagaggaacagacacagctcgcagacctctgaagagagtgccattgagacgggttccagcagttccaccttcatcaagagagaggacgagaccattgaagacatcgacatgatggacgacatcggcatagactcttcagacctggtggaagacagcttcctgtaactggcggattcgaggggttccttccacttctggggccacctctggatcccgttcagaaaaccactttattgcaatgcggaggttgagaggaggacttggttgatgtttaaagagaagttcccagccaagggcctcggggagcgttctaaatatgaatgaatgggatattttgaaatgaactttgtcagtgttgcctcttgcaatgcctcagtagcatctcagtggtgtgtgaagtttggagatagatggataagggaataataggccacagaaggtgaactttgtgcttcaaggacattggtgagagtccaacagacacaatttatactgcgacagaacttcagcattgtaattatgtaaataactctaaccaaggctgtgtttagattgtattaactatcttctttggacttctgaagagaccactcaatccatccatgtacttccctcttgaaacctgatgtcagctgctgttgaactttttaaagaagtgcatgaaaaaccatttttgaaccttaaaaggtactggtactatagcattttgctatcttttttagtgttaaagagataaagaataataattaaccaaccttgtttaatagatttgggtcatttagaagcctgacaactcattttcatattgtaatctatgtttataatactactactgttatcagtaatgctaaatgtgtaataatgtaacatgatttccctccagagaaagcacaatttaaaacaatccttactaagtaggtgatgagtttgacagtttttgacatttatattaaataacatgtttctctataaagtatggtaatagctttagtgaattaaatttagttgagcatagagaacaaagtaaaagtagtgttgtccaggaagtcagaatttttaactgtactgaataggttccccaatccatcgtattaaaaaacaattaactgccctctgaaataatgggattagaaacaaacaaaactcttaagtcctaaaagttctcaatgtagaggcataaacctgtgctgaacataacttctcatgtatattacccaatggaaaatataatgatcagcaaaaagactggatttgcagaagttttttttttttttttcttcatgcctgatgaaagctttggcgaccccaatatatgtattttttgaatctatgaacctgaaaagggtcagaaggatgcccagacatcagcctccttctttcaccccttaccccaaagagaaagagtttgaaactcgagaccataaagatattctttagtggaggctggatgtgcattagcctggatcctcagttctcaaatgtgtgtggcagccaggatgactagatcctgggtttccatccttgagattctgaagtatgaagtctgagggaaaccagagtctgtatttttctaaactccctggctgttctgatcggccagttttcggaaacactgacttaggtttcaggaagttgccatgggaaacaaataatttgaactttggaacagggttggaattcaaccacgcaggaagcctactatttaaatccttggcttcaggttagtgacatttaatgccatctagctagcaattgcgaccttaatttaactttccagtcttagctgaggctgagaaagctaaagtttggttttgacaggttttccaaaagtaaagatgctacttcccactgtatgggggagattgaactttccccgtctcccgtcttctgcctcccactccataccccgccaaggaaaggcatgtacaaaaattatgcaattcagtgttccaagtctctgtgtaaccagctcagtgttttggtggaaaaaacattttaagttttactgataatttgaggttagatgggaggatgaattgtcacatctatccacactgtcaaacaggttggtgtgggttcattggcattctttgcaatactgcttaattgctgataccatatgaatgaaacatgggctgtgattactgcaatcactgtgctatcggcagatgatgctttggaagatgcagaagcaataataaagtacttgactacctactggtgtaatctcaatgcaagccccaactttcttatccaactttttcatagtaagtgcgaagactgagccagattggccaattaaaaacgaaaacctgactaggttctgtagagccaattagacttgaaatacgtttgtgtttctagaatcacagctcaagcattctgtttatcgctcactctcccttgtacagccttattttgttggtgctttgcattttgatattgctgtgagccttgcatgacatcatgaggccggatgaaacttctcagtccagcagtttccagtcctaacaaatgctcccacctgaatttgtatatgactgcatttgtgtgtgtgtgtgtgttttcagcaaattccagatttgtttccttttggcctcctgcaaagtctccagaagaaaatttgccaatctttcctactttctatttttatgatgacaatcaaagccggcctgagaaacactatttgtgactttttaaacgattagtgatgtccttaaaatgtggtctgccaatctgtacaaaatggtcctatttttgtgaagagggacataagataaaatgatgttatacatcaatatgtatatatgtatttctatatagacttggagaatactgccaaaacatttatgacaagctgtatcactgccttcgtttatatttttttaactgtgataatccccacaggcacattaactgttgcacttttgaatgtccaaaatttatattttagaaataataaaaagaaagatacttacatgttcccaaaacaatggtgtggtgaatgtgtgagaaaaactaacttgatagggtctaccaatacaaaatgtattacgaatgcccctgttcatgtttttgttttaaaacgtgtaaatgaagatctttatatttcaataaatgatatataatttaaagttaaaaaaaaaaaaaaa

SEQ ID NO: 4 - DNA, 5718 bp, Homo sapiens
ctcctgaggctgccagcagccagcagtgactgcccgccctatctgggacccaggatcgctctgtgagcaacttggagccagagaggagatcaacaaggaggaggagagagccggcccctcagccctgctgcccagcagcagcctgtgctcgccctgcccaacgcagacagccagacccagggcggcccctctggcggctctgctcctcccgaaggatgcttggggagtgaggcgaagctgggccgctcctctcccctacagcagcccccttcctccatccctctgttctcctgagccttcaggagcctgcaccagtcctgcctgtccttctactcagctgttacccactctgggaccagcagtctttctgataactgggagagggcagtaaggaggacttcctggagggggtgactgtccagagcctggaactgtgcccacaccagaagccatcagcagcaaggacaccatgcggcttccgggtgcgatgccagctctggccctcaaaggcgagctgctgttgctgtctctcctgttacttctggaaccacagatctctcagggcctggtcgtcacacccccggggccagagcttgtcctcaatgtctccagcaccttcgttctgacctgctcgggttcagctccggtggtgtgggaacggatgtcccaggagcccccacaggaaatggccaaggcccaggatggcaccttctccagcgtgctcacactgaccaacctcactgggctagacacgggagaatacttttgcacccacaatgactcccgtggactggagaccgatgagcggaaacggctctacatctttgtgccagatcccaccgtgggcttcctccctaatgatgccgaggaactattcatctttctcacggaaataactgagatcaccattccatgccgagtaacagacccacagctggtggtgacactgcacgagaagaaaggggacgttgcactgcctgtcccctatgatcaccaacgtggcttttctggtatctttgaggacagaagctacatctgcaaaaccaccattggggacagggaggtggattctgatgcctactatgtctacagactccaggtgtcatccatcaacgtctctgtgaacgcagtgcagactgtggtccgccagggtgagaacatcaccctcatgtgcattgtgatcgggaatgaggtggtcaacttcgagtggacatacccccgcaaagaaagtgggcggctggtggagccggtgactgacttcctcttggatatgccttaccacatccgctccatcctgcacatccccagtgccgagttagaagactcggggacctacacctgcaatgtgacggagagtgtgaatgaccatcaggatgaaaaggccatcaacatcaccgtggttgagagcggctacgtgcggctcctgggagaggtgggcacactacaatttgctgagctgcatcggagccggacactgcaggtagtgttcgaggcctacccaccgcccactgtcctgtggttcaaagacaaccgcaccctgggcgactccagcgctggcgaaatcgccctgtccacgcgcaacgtgtcggagacccggtatgtgtcagagctgacactggttcgcgtgaaggtggcagaggctggccactacaccatgcgggccttccatgaggatgctgaggtccagctctccttccagctacagatcaatgtccctgtccgagtgctggagctaagtgagagccaccctgacagtggggaacagacagtccgctgtcgtggccggggcatgccccagccgaacatcatctggtctgcctgcagagacctcaaaaggtgtccacgtgagctgccgcccacgctgctggggaacagttccgaagaggagagccagctggagactaacgtgacgtactgggaggaggagcaggagtttgaggtggtgagcacactgcgtctgcagcacgtggatcggccactgtcggtgcgctgcacgctgcgcaacgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttacgagatccgatggaaggtgattgagtctgtgagctctgacggccatgagtacatctacgtggaccccatgcagctgccctatgactccacgtgggagctgccgcgggaccagcttgtgctgggacgcaccctcggctctggggcctttgggcaggtggtggaggccacggctcatggcctgagccattctcaggccacgatgaaagtggccgtcaagatgcttaaatccacagcccgcagcagtgagaagcaagcccttatgtcggagctgaagatcatgagtcaccttgggccccacctgaacgtggtcaacctgttgggggcctgcaccaaaggaggacccatctatatcatcactgagtactgccgctacggagacctggtggactacctgcaccgcaacaaacacaccttcctgcagcaccactccgacaagcgccgcccgcccagcgcggagctctacagcaatgctctgcccgttgggctccccctgcccagccatgtgtccttgaccggggagagcgacggtggctacatggacatgagcaaggacgagtcggtggactatgtgcccatgctggacatgaaaggagacgtcaaatatgcagacatcgagtcctccaactacatggccccttacgataactacgttccctctgcccctgagaggacctgccgagcaactttgatcaacgagtctccagtgctaagctacatggacctcgtgggcttcagctaccaggtggccaatggcatggagtttctggcctccaagaactgcgtccacagagacctggcggctaggaacgtgctcatctgtgaaggcaagctggtcaagatctgtgactttggcctggctcgagacatcatgcgggactcgaattacatctccaaaggcagcacctttttgcctttaaagtggatggctccggagagcatcttcaacagcctctacaccaccctgagcgacgtgtggtccttcgggatcctgctctgggagatcttcaccttgggtggcaccccttacccagagctgcccatgaacgagcagttctacaatgccatcaaacggggttaccgcatggcccagcctgcccatgcctccgacgagatctatgagatcatgcagaagtgctgggaagagaagtttgagattcggccccccttctcccagctggtgctgcttctcgagagactgttgggcgaaggttacaaaaagaagtaccagcaggtggatgaggagtttctgaggagtgaccacccagccatccttcggtcccaggcccgcttgcctgggttccatggcctccgatctcccctggacaccagctccgtcctctatactgccgtgcagcccaatgagggtgacaacgactatatcatccccctgcctgaccccaaacccgaggttgctgacgagggcccactggagggttcccccagcctagccagctccaccctgaatgaagtcaacacctcctcaaccatctcctgtgacagccccctggagccccaggacgaaccagagccagagccccagcttgagctccaggtggagccggagccagagctggaacagttgccggattcggggtgccctgcgcctcgggcggaagcagaggatagcttcctgtagggggctggcccctaccctgccctgcctgaagctccccccctgccagcacccagcatctcctggcctggcctgaccgggcttcctgtcagccaggctgcccttatcagctgtccccttctggaagctttctgctcctgacgtgttgtgccccaaaccctggggctggcttaggaggcaagaaaactgcaggggccgtgaccagccctctgcctccagggaggccaactgactctgagccagggttcccccagggaactcagttttcccatatgtaagatgggaaagttaggcttgatgacccagaatctaggattctctccctggctgacaggtggggagaccgaatccctccctgggaagattcttggagttactgaggtggtaaattaacttttttctgttcagccagctacccctcaaggaatcatagctctctcctcgcacttttatccacccaggagctagggaagagaccctagcctccctggctgctggctgagctagggcctagccttgagcagtgttgcctcatccagaagaaagccagtctcctccctatgatgccagtccctgcgttccctggcccgagctggtctggggccattaggcagcctaattaatgctggaggctgagccaagtacaggacacccccagcctgcagcccttgcccagggcacttggagcacacgcagccatagcaagtgcctgtgtccctgtccttcaggcccatcagtcctggggctttttctttatcaccctcagtcttaatccatccaccagagtctagaaggccagacgggccccgcatctgtgatgagaatgtaaatgtgccagtgtggagtggccacgtgtgtgtgccagtatatggccctggctctgcattggacctgctatgaggctttggaggaatccctcaccctctctgggcctcagtttccccttcaaaaaatgaataagtcggacttattaactctgagtgccttgccagcactaacattctagagtattccaggtggttgcacatttgtccagatgaagcaaggccatataccctaaacttccatcctgggggtcagctgggctcctgggagattccagatcacacatcacactctggggactcaggaaccatgccccttccccaggcccccagcaagtctcaagaacacagctgcacaggccttgacttagagtgacagccggtgtcctggaaagcccccagcagctgccccagggacatgggaagaccacgggacctctttcactacccacgatgacctccgggggtatcctgggcaaaagggacaaagagggcaaatgagatcacctcctgcagcccaccactccagcacctgtgccgaggtctgcgtcgaagacagaatggacagtgaggacagttatgtcttgtaaaagacaagaagcttcagatgggtaccccaagaaggatgtgagaggtgggcgctttggaggtttgcccctcacccaccagctgccccatccctgaggcagcgctccatgggggtatggttttgtcactgcccagacctagcagtgacatctcattgtccccagcccagtgggcattggaggtgccaggggagtcagggttgtagccaagacgcccccgcacggggagggttgggaagggggtgcaggaagctcaacccctctgggcaccaaccctgcattgcaggttggcaccttacttccctgggatccccagagttggtccaaggagggagagtgggttctcaatacggtaccaaagatataatcacctaggtttacaaatatttttaggactcacgttaactcacatttatacagcagaaatgctattttgtatgctgttaagtttttctatctgtgtacttttttttaagggaaagattttaatattaaacctggtgcttctcactcacaaaaa

SEQ ID NO: 5 - DNA, 19 bp, Artificial
  Target Sequence
gaaagttaccagtctatta

SEQ ID NO: 6 - DNA, 21 bp, Artificial
  sense strand with 3′NN
gaaaguuaccagucuauuann

SEQ ID NO: 7 - DNA, 21 bp, Artificial
  Antisense strand with 3′NN
uaauagacugguaacuuucnn

SEQ ID NO: 8 - RNA, 21 nt, Artificial
  Sense Strand
gaaaguuaccagucuauuauu

SEQ ID NO: 9 - RNA, 21 nt, Artificial
  Antisense Strand
uaauagacugguaacuuucuu

SEQ ID NO: 10 - RNA, 19 nt, Artificial
  Sense Strand
gaaaguuaccagucuauua

SEQ ID NO: 11 - RNA, 19 nt, Artificial
  Antisense Strand
uaauagacugguaacuuuc

SEQ ID NO: 12 - DNA, 48 bp, Artificial
  Hairpin duplex with loop
gaaaguuaccagucuauuannnnnnnnuaauagacugguaacuuucuu

SEQ ID NO: 13 - DNA, 25 bp, Artificial
  Sense Strand
gaaagttaccagtctattatgtaca

SEQ ID NO: 14 - RNA, 25 nt, Artificial
  Sense Strand
gaaaguuaccagucuauuauguaca

SEQ ID NO: 15 - RNA, 27 nt, Artificial
  Antisense Strand
uguacauaauagacugguaacuuucuu

SEQ ID NO: 16 - DNA, 19 bp, Artificial
  Target Sequence
gaaagttaccagtctatta

SEQ ID NO: 17 - DNA, 19 bp, Artificial
  Target Sequence
gtacatagttgtcgttgta

SEQ ID NO: 18 - DNA, 19 bp, Artificial
  Target Sequence
tccgtctcatggaattgaa

SEQ ID NO: 19 - DNA, 19 bp, Artificial
  Target Sequence
agcaagaactgaactaaat

SEQ ID NO: 20 - DNA, 19 bp, Artificial
  Target Sequence
tcagcataagaaacttgta

SEQ ID NO: 21 - DNA, 19 bp, Artificial
  Target Sequence
tgagcaccttaactataga

SEQ ID NO: 22 - DNA, 19 bp, Artificial
  Target Sequence
ggcatgtactgacgattat

SEQ ID NO: 23 - DNA, 19 bp, Artificial
  Target Sequence
actcaggcattgtattgaa

SEQ ID NO: 24 - DNA, 19 bp, Artificial
  Target Sequence
ggatgaacattgtgaacga

SEQ ID NO: 25 - DNA, 19 bp, Artificial
  Target Sequence
gtgaacgactgccttatga

SEQ ID NO: 26 - DNA, 19 bp, Artificial
  Target Sequence
caagatcctcattcatatt

SEQ ID NO: 27 - DNA, 19 bp, Artificial
  Target Sequence
ttggaaacctgtccactta

SEQ ID NO: 28 - DNA, 19 bp, Artificial
  Target Sequence
ttcttggcatcgcgaaagt

SEQ ID NO: 29 - DNA, 19 bp, Artificial
  Target Sequence
atatcctcttatcggagaa

SEQ ID NO: 30 - DNA, 19 bp, Artificial
  Target Sequence
ggagaagaacgtggttaaa

SEQ ID NO: 31 - DNA, 19 bp, Artificial
  Target Sequence
ggaaatctcttgcaagcta

SEQ ID NO: 32 - DNA, 19 bp, Artificial
  Target Sequence
gaaatctcttgcaagctaa

SEQ ID NO: 33 - DNA, 19 bp, Artificial
  Target Sequence
ctacattgttcttccgata

SEQ ID NO: 34 - DNA, 19 bp, Artificial
  Target Sequence
gtatggttcttgcctcaga

SEQ ID NO: 35 - DNA, 19 bp, Artificial
  Target Sequence
gatagagattggagtgcaa

SEQ ID NO: 36 - DNA, 19 bp, Artificial
  Target Sequence
gagctctcctcctgtttaa

SEQ ID NO: 37 - DNA, 19 bp, Artificial
  Target Sequence
gcaggaagtagccgcattt

SEQ ID NO: 38 - DNA, 19 bp, Artificial
  Target Sequence
ttcatttcgacaacagaaa

SEQ ID NO: 39 - DNA, 19 bp, Artificial
  Target Sequence
agccagtcttctaggcata

SEQ ID NO: 40 - DNA, 19 bp, Artificial
  Target Sequence
ctaggcatatcctggaaga

SEQ ID NO: 41 - DNA, 19 bp, Artificial
  Target Sequence
agataaaccaggcaacgta

SEQ ID NO: 42 - DNA, 19 bp, Artificial
  Target Sequence
tgatagaaaggaagactaa

SEQ ID NO: 43 - DNA, 19 bp, Artificial
  Target Sequence
gaaaggaagactaacgtta

SEQ ID NO: 44 - DNA, 19 bp, Artificial
  Target Sequence
aacgttaccttgctttgga

SEQ ID NO: 45 - DNA, 19 bp, Artificial
  Target Sequence
tgctgtttctgactcctaa

SEQ ID NO: 46 - DNA, 19 bp, Artificial
  Target Sequence
ctaatgagagttccttcca

SEQ ID NO: 47 - DNA, 19 bp, Artificial
  Target Sequence
gaaaggacattcagctcaa

SEQ ID NO: 48 - DNA, 19 bp, Artificial
  Target Sequence
gacatgctatggcacatat

SEQ ID NO: 49 - DNA, 19 bp, Artificial
  Target Sequence
gcataacaaaggtcataat

SEQ ID NO: 50 - DNA, 19 bp, Artificial
  Target Sequence
agatcaatggtgcttattt

SEQ ID NO: 51 - DNA, 19 bp, Artificial
  Target Sequence
ctaccagctactttaacta

SEQ ID NO: 52 - DNA, 19 bp, Artificial
  Target Sequence
gtactcggccaggtatata

SEQ ID NO: 53 - DNA, 19 bp, Artificial
  Target Sequence
gccgctacctactaatgaa

SEQ ID NO: 54 - DNA, 19 bp, Artificial
  Target Sequence
gctacctactaatgaagaa

SEQ ID NO: 55 - DNA, 19 bp, Artificial
  Target Sequence
ccttcaacatttctgttaa

SEQ ID NO: 56 - DNA, 19 bp, Artificial
  Target Sequence
ctgttaaagttcttccaaa

SEQ ID NO: 57 - DNA, 19 bp, Artificial
  Target Sequence
ccaagaagcttctatacaa

SEQ ID NO: 58 - DNA, 19 bp, Artificial
  Target Sequence
caagaagcttctatacaaa

SEQ ID NO: 59 - DNA, 19 bp, Artificial
  Target Sequence
aaagtcagaccactctaaa

SEQ ID NO: 60 - DNA, 19 bp, Artificial
  Target Sequence
tgacctggcaaccaatatt

SEQ ID NO: 61 - DNA, 19 bp, Artificial
  Target Sequence
agtgatcagcagaatatta

SEQ ID NO: 62 - DNA, 19 bp, Artificial
  Target Sequence
gtgatcagcagaatattaa

SEQ ID NO: 63 - DNA, 19 bp, Artificial
  Target Sequence
ttgacttcggtgctactta

SEQ ID NO: 64 - DNA, 19 bp, Artificial
  Target Sequence
tgacttcggtgctacttaa

SEQ ID NO: 65 - DNA, 19 bp, Artificial
  Target Sequence
ggtgctacttaacaactta

SEQ ID NO: 66 - DNA, 19 bp, Artificial
  Target Sequence
gtgatttcttggacaatat

SEQ ID NO: 67 - DNA, 19 bp, Artificial
  Target Sequence
ggctattctatttcttcta

SEQ ID NO: 68 - DNA, 19 bp, Artificial
  Target Sequence
tctattactatccgttaca

SEQ ID NO: 69 - DNA, 19 bp, Artificial
  Target Sequence
ctattactatccgttacaa

SEQ ID NO: 70 - DNA, 19 bp, Artificial
  Target Sequence
gcacgttgatgtgaagata

SEQ ID NO: 71 - DNA, 19 bp, Artificial
  Target Sequence
acgttgatgtgaagataaa

SEQ ID NO: 72 - DNA, 19 bp, Artificial
  Target Sequence
ggaatgacatcaaatttca

SEQ ID NO: 73 - DNA, 19 bp, Artificial
  Target Sequence
atggactacttgagccaaa

SEQ ID NO: 74 - DNA, 19 bp, Artificial
  Target Sequence
ccatcgagtcactgaatta

SEQ ID NO: 75 - DNA, 19 bp, Artificial
  Target Sequence
tgtatgatctaatgagaca

SEQ ID NO: 76 - DNA, 19 bp, Artificial
  Target Sequence
agatattggtgtccttaaa

SEQ ID NO: 77 - DNA, 19 bp, Artificial
  Target Sequence
atattggtgtccttaaaca

SEQ ID NO: 78 - DNA, 19 bp, Artificial
  Target Sequence
cgaaagacctacgtgaata

SEQ ID NO: 79 - DNA, 19 bp, Artificial
  Target Sequence
gccaaaggatgtgatatat

SEQ ID NO: 80 - DNA, 19 bp, Artificial
  Target Sequence
gtgtacatatgtgctggaa

SEQ ID NO: 81 - DNA, 19 bp, Artificial
  Target Sequence
gtacatatgtgctggaatt

SEQ ID NO: 82 - DNA, 19 bp, Artificial
  Target Sequence
tgtgctggaattctaacaa

SEQ ID NO: 83 - DNA, 19 bp, Artificial
  Target Sequence
ctaacaagtcataggttaa

SEQ ID NO: 84 - DNA, 19 bp, Artificial
  Target Sequence
tcagtccaggatgctaaca

SEQ ID NO: 85 - DNA, 19 bp, Artificial
  Target Sequence
ctggtaatattgacttgta

SEQ ID NO: 86 - DNA, 19 bp, Artificial
  Target Sequence
ggagacatgtgacatttat

SEQ ID NO: 87 - DNA, 19 bp, Artificial
  Target Sequence
gttgtgagtttaccttgta

SEQ ID NO: 88 - DNA, 19 bp, Artificial
  Target Sequence
tgtgagtttaccttgtata

SEQ ID NO: 89 - DNA, 19 bp, Artificial
  Target Sequence
aaatgtcttgcctactcaa

SEQ ID NO: 90 - DNA, 19 bp, Artificial
  Target Sequence
gcaggcacatttacatcta

SEQ ID NO: 91 - DNA, 19 bp, Artificial
  Target Sequence
ctctaggaatgacggatta

SEQ ID NO: 92 - DNA, 19 bp, Artificial
  Target Sequence
tgatgattctgccattata

SEQ ID NO: 93 - DNA, 19 bp, Artificial
  Target Sequence
cgagactcctgtaacctta

SEQ ID NO: 94 - DNA, 19 bp, Artificial
  Target Sequence
gaaataaggtatcgaagca

SEQ ID NO: 95 - DNA, 19 bp, Artificial
  Target Sequence
aaggtatcgaagcaaatta

SEQ ID NO: 96 - DNA, 19 bp, Artificial
  Target Sequence
aggtatcgaagcaaattaa

SEQ ID NO: 97 - DNA, 19 bp, Artificial
  Target Sequence
ggtatcgaagcaaattaaa

SEQ ID NO: 98 - DNA, 19 bp, Artificial
  Target Sequence
gaagacagtggccattata

SEQ ID NO: 99 - DNA, 19 bp, Artificial
  Target Sequence
tggccattatactattgta

SEQ ID NO: 100 - DNA, 19 bp, Artificial
  Target Sequence
cacgccgcttcctgatatt

SEQ ID NO: 101 - DNA, 19 bp, Artificial
  Target Sequence
tgcgatgcctggctaagaa

SEQ ID NO: 102 - DNA, 19 bp, Artificial
  Target Sequence
ggaacagcctatggattaa

SEQ ID NO: 103 - DNA, 19 bp, Artificial
  Target Sequence
acacggagctatgttattt

SEQ ID NO: 104 - DNA, 19 bp, Artificial
  Target Sequence
ggagcgttctaaatatgaa

SEQ ID NO: 105 - DNA, 19 bp, Artificial
  Target Sequence
cactcaatccatccatgta

SEQ ID NO: 106 - DNA, 19 bp, Artificial
  Target Sequence
ccaaccttgtttaatagat

SEQ ID NO: 107 - DNA, 19 bp, Artificial
  Target Sequence
ctactactgttatcagtaa

SEQ ID NO: 108 - DNA, 19 bp, Artificial
  Target Sequence
agttgagcatagagaacaa

SEQ ID NO: 109 - DNA, 19 bp, Artificial
  Target Sequence
ttctcaatgtagaggcata

SEQ ID NO: 110 - DNA, 19 bp, Artificial
  Target Sequence
ctcaatgtagaggcataaa

SEQ ID NO: 111 - DNA, 19 bp, Artificial
  Target Sequence
ataaacctgtgctgaacat

SEQ ID NO: 112 - DNA, 19 bp, Artificial
  Target Sequence
ttgaaactcgagaccataa

SEQ ID NO: 113 - DNA, 19 bp, Artificial
  Target Sequence
tgaaactcgagaccataaa

SEQ ID NO: 114 - DNA, 19 bp, Artificial
  Target Sequence
ggaggctggatgtgcatta

SEQ ID NO: 115 - DNA, 19 bp, Artificial
  Target Sequence
ttcaggttagtgacattta

SEQ ID NO: 116 - DNA, 19 bp, Artificial
  Target Sequence
ctagcaattgcgaccttaa

SEQ ID NO: 117 - DNA, 19 bp, Artificial
  Target Sequence
tagcaattgcgaccttaat

SEQ ID NO: 118 - DNA, 19 bp, Artificial
  Target Sequence
ctgataatttgaggttaga

SEQ ID NO: 119 - DNA, 19 bp, Artificial
  Target Sequence
gatgaattgtcacatctat

SEQ ID NO: 120 - DNA, 19 bp, Artificial
  Target Sequence
tctttgcaatactgcttaa

SEQ ID NO: 121 - DNA, 19 bp, Artificial
  Target Sequence
cttaattgctgataccata

SEQ ID NO: 122 - DNA, 19 bp, Artificial
  Target Sequence
gaagatgcagaagcaataa

SEQ ID NO: 123 - DNA, 19 bp, Artificial
  Target Sequence
agtttccagtcctaacaaa

SEQ ID NO: 124 - DNA, 19 bp, Artificial
  Target Sequence
atcactgccttcgtttata

SEQ ID NO: 125 - DNA, 19 bp, Artificial
  Target Sequence
ggaaacggctctacatctt

SEQ ID NO: 126 - DNA, 19 bp, Artificial
  Target Sequence
gaaacggctctacatcttt

SEQ ID NO: 127 - DNA, 19 bp, Artificial
  Target Sequence
gatgccgaggaactattca

SEQ ID NO: 128 - DNA, 19 bp, Artificial
  Target Sequence
atctttctcacggaaataa

SEQ ID NO: 129 - DNA, 19 bp, Artificial
  Target Sequence
tcacggaaataactgagat

SEQ ID NO: 130 - DNA, 19 bp, Artificial
  Target Sequence
accattccatgccgagtaa

SEQ ID NO: 131 - DNA, 19 bp, Artificial
  Target Sequence
tggtgacactgcacgagaa

SEQ ID NO: 132 - DNA, 19 bp, Artificial
  Target Sequence
tggattctgatgcctacta

SEQ ID NO: 133 - DNA, 19 bp, Artificial
  Target Sequence
tcaacttcgagtggacata

SEQ ID NO: 134 - DNA, 19 bp, Artificial
  Target Sequence
tgacggagagtgtgaatga

SEQ ID NO: 135 - DNA, 19 bp, Artificial
  Target Sequence
ccttccagctacagatcaa

SEQ ID NO: 136 - DNA, 19 bp, Artificial
  Target Sequence
cgatgaaagtggccgtcaa

SEQ ID NO: 137 - DNA, 19 bp, Artificial
  Target Sequence
caacgagtctccagtgcta

SEQ ID NO: 138 - DNA, 19 bp, Artificial
  Target Sequence
ggaacgtgctcatctgtga

SEQ ID NO: 139 - DNA, 19 bp, Artificial
  Target Sequence
tcaaccatctcctgtgaca

SEQ ID NO: 140 - DNA, 19 bp, Artificial
  Target Sequence
tggcttaggaggcaagaaa

SEQ ID NO: 141 - DNA, 19 bp, Artificial
  Target Sequence
tactgaggtggtaaattaa

SEQ ID NO: 142 - DNA, 19 bp, Artificial
  Target Sequence
ccattaggcagcctaatta

SEQ ID NO: 143 - DNA, 19 bp, Artificial
  Target Sequence
gaataagtcggacttatta

SEQ ID NO: 144 - DNA, 19 bp, Artificial
  Target Sequence
tgccagcactaacattcta

SEQ ID NO: 145 - DNA, 19 bp, Artificial
  Target Sequence
cactaacattctagagtat

SEQ ID NO: 146 - DNA, 19 bp, Artificial
  Target Sequence
gattccagatcacacatca

SEQ ID NO: 147 - DNA, 19 bp, Artificial
  Target Sequence
ggacagttatgtcttgtaa

SEQ ID NO: 148 - DNA, 19 bp, Artificial
  Target Sequence
gacagttatgtcttgtaaa

SEQ ID NO: 149 - DNA, 19 bp, Artificial
  Target Sequence
attgcaggttggcacctta

SEQ ID NO: 150 - DNA, 19 bp, Artificial
  Target Sequence
tgcaggttggcaccttact

SEQ ID NO: 151 - DNA, 19 bp, Artificial
  Target Sequence
ggttctcaatacggtacca

SEQ ID NO: 152 - DNA, 19 bp, Artificial
  Target Sequence
ttctcaatacggtaccaaa

SEQ ID NO: 153 - DNA, 19 bp, Artificial
  Target Sequence
ctcaatacggtaccaaaga

SEQ ID NO: 154 - DNA, 19 bp, Artificial
  Target Sequence
tcaatacggtaccaaagat

SEQ ID NO: 155 - DNA, 19 bp, Artificial
  Target Sequence
caatacggtaccaaagata

SEQ ID NO: 156 - DNA, 19 bp, Artificial
  Target Sequence
atacggtaccaaagatata

SEQ ID NO: 157 - DNA, 19 bp, Artificial
  Target Sequence
tacggtaccaaagatataa

SEQ ID NO: 158 - DNA, 19 bp, Artificial
  Target Sequence
acggtaccaaagatataat

SEQ ID NO: 159 - DNA, 19 bp, Artificial
  Target Sequence
ataatcacctaggtttaca

SEQ ID NO: 160 - DNA, 19 bp, Artificial
  Target Sequence
taatcacctaggtttacaa

SEQ ID NO: 161 - DNA, 19 bp, Artificial
  Target Sequence
cacctaggtttacaaatat

SEQ ID NO: 162 - DNA, 19 bp, Artificial
  Target Sequence
ggactcacgttaactcaca

SEQ ID NO: 163 - DNA, 19 bp, Artificial
  Target Sequence
tggtgcttctcactcacaa

SEQ ID NO: 164 - DNA, 19 bp, Artificial
  Target Sequence
ttgtaaaccgagacctaaa

SEQ ID NO: 165 - DNA, 19 bp, Artificial
  Target Sequence
cagtacggcaccactcaaa

SEQ ID NO: 166 - DNA, 19 bp, Artificial
  Target Sequence
atgtgaagcggtcaacaaa

SEQ ID NO: 167 - DNA, 19 bp, Artificial
  Target Sequence
gttctctaatagcacaaat

SEQ ID NO: 168 - DNA, 19 bp, Artificial
  Target Sequence
gagaatcagacgacaagta

SEQ ID NO: 169 - DNA, 19 bp, Artificial
  Target Sequence
ggctacttcttgtcatcat

SEQ ID NO: 170 - DNA, 19 bp, Artificial
  Target Sequence
tcatcctacggaccgttaa

SEQ ID NO: 171 - DNA, 19 bp, Artificial
  Target Sequence
acgtttggcagaacttgta

SEQ ID NO: 172 - DNA, 19 bp, Artificial
  Target Sequence
gccagatcatatagaagta

SEQ ID NO: 173 - DNA, 19 bp, Artificial
  Target Sequence
agaagtaaacagtggtaaa

SEQ ID NO: 174 - DNA, 19 bp, Artificial
  Target Sequence
gctggccgctacctactaa

SEQ ID NO: 175 - DNA, 19 bp, Artificial
  Target Sequence
attataccttgtcgcacaa

SEQ ID NO: 176 - DNA, 19 bp, Artificial
  Target Sequence
agactcctgtaaccttaca

SEQ ID NO: 177 - DNA, 19 bp, Artificial
  Target Sequence
gcatcacaatgctggaaga

SEQ ID NO: 178 - DNA, 19 bp, Artificial
  Target Sequence
tcacaatgctggaagaaat

SEQ ID NO: 179 - DNA, 19 bp, Artificial
  Target Sequence
caacctgcatgaagtcaaa

SEQ ID NO: 180 - DNA, 19 bp, Artificial
  Target Sequence
gatattgagtggatgatat

SEQ ID NO: 181 - DNA, 19 bp, Artificial
  Target Sequence
tgtctgaactgaagataat

SEQ ID NO: 182 - DNA, 19 bp, Artificial
  Target Sequence
gatcgtccagcctcatata

SEQ ID NO: 183 - DNA, 19 bp, Artificial
  Target Sequence
atcgtccagcctcatataa

SEQ ID NO: 184 - DNA, 19 bp, Artificial
  Target Sequence
tctacgagatcatggtgaa

SEQ ID NO: 185 - DNA, 19 bp, Artificial
  Target Sequence
ggaacagtgagccggagaa

SEQ ID NO: 186 - DNA, 19 bp, Artificial
  Target Sequence
atcattcctctgcctgaca

SEQ ID NO: 187 - DNA, 19 bp, Artificial
  Target Sequence
ttgaagacatcgacatgat

SEQ ID NO: 188 - DNA, 19 bp, Artificial
  Target Sequence
tggacgacatcggcataga

SEQ ID NO: 189 - DNA, 19 bp, Artificial
  Target Sequence
cttcagacctggtggaaga

SEQ ID NO: 190 - DNA, 19 bp, Artificial
  Target Sequence
tggagactaacgtgacgta

SEQ ID NO: 191 - DNA, 19 bp, Artificial
  Target Sequence
acggccatgagtacatcta

SEQ ID NO: 192 - DNA, 19 bp, Artificial
  Target Sequence
attctcaggccacgatgaa

SEQ ID NO: 193 - DNA, 19 bp, Artificial
  Target Sequence
ggccgtcaagatgcttaaa

SEQ ID NO: 194 - DNA, 19 bp, Artificial
  Target Sequence
gccgtcaagatgcttaaat

SEQ ID NO: 195 - DNA, 19 bp, Artificial
  Target Sequence
tggactacctgcaccgcaa

SEQ ID NO: 196 - DNA, 19 bp, Artificial
  Target Sequence
gaaaggagacgtcaaatat

SEQ ID NO: 197 - DNA, 19 bp, Artificial
  Target Sequence
gacgtcaaatatgcagaca

SEQ ID NO: 198 - DNA, 19 bp, Artificial
  Target Sequence
cagacatcgagtcctccaa

SEQ ID NO: 199 - DNA, 19 bp, Artificial
  Target Sequence
gccgagcaactttgatcaa

SEQ ID NO: 200 - DNA, 19 bp, Artificial
  Target Sequence
caagaactgcgtccacaga

SEQ ID NO: 201 - DNA, 19 bp, Artificial
  Target Sequence
actcgaattacatctccaa

SEQ ID NO: 202 - DNA, 19 bp, Artificial
  Target Sequence
ctcgaattacatctccaaa

SEQ ID NO: 203 - DNA, 19 bp, Artificial
  Target Sequence
ccatgaacgagcagttcta

SEQ ID NO: 204 - DNA, 19 bp, Artificial
  Target Sequence
atgcctccgacgagatcta

SEQ ID NO: 205 - DNA, 5777 bp, Homo sapiens
gcggacactcctctcggctcctccccggcagcggcggcggctcggagcgggctccggggctcgggtgcagcggccagcgggcctggcggcgaggattacccggggaagtggttgtctcctggctggagccgcgagacgggcgctcagggcgcggggccggcggcggcgaacgagaggacggactctggcggccgggtcgttggccgggggagcgcgggcaccgggcgagcaggccgcgtcgcgctcaccatggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctttatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagagatcaggaagcaccatacctcctgcgaaacctcagtgatcacacagtggccatcagcagttccaccactttagactgtcatgctaatggtgtccccgagcctcagatcacttggtttaaaaacaaccacaaaatacaacaagagcctggaattattttaggaccaggaagcagcacgctgtttattgaaagagtcacagaagaggatgaaggtgtctatcactgcaaagccaccaaccagaagggctctgtggaaagttcagcatacctcactgttcaaggaacctcggacaagtctaatctggagctgatcactctaacatgcacctgtgtggctgcgactctcttctggctcctattaaccctccttatccgaaaaatgaaaaggtcttcttctgaaataaagactgactacctatcaattataatggacccagatgaagttcctttggatgagcagtgtgagcggctcccttatgatgccagcaagtgggagtttgcccgggagagacttaaactgggcaaatcacttggaagaggggcttttggaaaagtggttcaagcatcagcatttggcattaagaaatcacctacgtgccggactgtggctgtgaaaatgctgaaagagggggccacggccagcgagtacaaagctctgatgactgagctaaaaatcttgacccacattggccaccatctgaacgtggttaacctgctgggagcctgcaccaagcaaggagggcctctgatggtgattgttgaatactgcaaatatggaaatctctccaactacctcaagagcaaacgtgacttattttttctcaacaaggatgcagcactacacatggagcctaagaaagaaaaaatggagccaggcctggaacaaggcaagaaaccaagactagatagcgtcaccagcagcgaaagctttgcgagctccggctttcaggaagataaaagtctgagtgatgttgaggaagaggaggattctgacggtttctacaaggagcccatcactatggaagatctgatttcttacagttttcaagtggccagaggcatggagttcctgtcttccagaaagtgcattcatcgggacctggcagcgagaaacattcttttatctgagaacaacgtggtgaagatttgtgattttggccttgcccgggatatttataagaaccccgattatgtgagaaaaggagatactcgacttcctctgaaatggatggctcccgaatctatctttgacaaaatctacagcaccaagagcgacgtgtggtcttacggagtattgctgtgggaaatcttctccttaggtgggtctccatacccaggagtacaaatggatgaggacttttgcagtcgcctgagggaaggcatgaggatgagagctcctgagtactctactcctgaaatctatcagatcatgctggactgctggcacagagacccaaaagaaaggccaagatttgcagaacttgtggaaaaactaggtgatttgcttcaagcaaatgtacaacaggatggtaaagactacatcccaatcaatgccatactgacaggaaatagtgggtttacatactcaactcctgccttctctgaggacttcttcaaggaaagtatttcagctccgaagtttaattcaggaagctctgatgatgtcagatatgtaaatgctttcaagttcatgagcctggaaagaatcaaaacctttgaagaacttttaccgaatgccacctccatgtttgatgactaccagggcgacagcagcactctgttggcctctcccatgctgaagcgcttcacctggactgacagcaaacccaaggcctcgctcaagattgacttgagagtaaccagtaaaagtaaggagtcggggctgtctgatgtcagcaggcccagtttctgccattccagctgtgggcacgtcagcgaaggcaagcgcaggttcacctacgaccacgctgagctggaaaggaaaatcgcgtgctgctccccgcccccagactacaactcggtggtcctgtactccaccccacccatctagagtttgacacgaagccttatttctagaagcacatgtgtatttatacccccaggaaactagcttttgccagtattatgcatatataagtttacacctttatctttccatgggagccagctgctttttgtgatttttttaatagtgctttttttttttgactaacaagaatgtaactccagatagagaaatagtgacaagtgaagaacactactgctaaatcctcatgttactcagtgttagagaaatccttcctaaacccaatgacttccctgctccaacccccgccacctcagggcacgcaggaccagtttgattgaggagctgcactgatcacccaatgcatcacgtaccccactgggccagccctgcagcccaaaacccagggcaacaagcccgttagccccaggggatcactggctggcctgagcaacatctcgggagtcctctagcaggcctaagacatgtgaggaggaaaaggaaaaaaagcaaaaagcaagggagaaaagagaaaccgggagaaggcatgagaaagaatttgagacgcaccatgtgggcacggagggggacggggctcagcaatgccatttcagtggcttcccagctctgacccttctacatttgagggcccagccaggagcagatggacagcgatgaggggacattttctggattctgggaggcaagaaaaggacaaatatcttttttggaactaaagcaaattttagacctttacctatggaagtggttctatgtccattctcattcgtggcatgttttgatttgtagcactgagggtggcactcaactctgagcccatacttttggctcctctagtaagatgcactgaaaacttagccagagttaggttgtctccaggccatgatggccttacactgaaaatgtcacattctattttgggtattaatatatagtccagacacttaactcaatttcttggtattattctgttttgcacagttagttgtgaaagaaagctgagaagaatgaaaatgcagtcctgaggagagttttctccatatcaaaacgagggctgatggaggaaaaaggtcaataaggtcaagggaagaccccgtctctataccaaccaaaccaattcaccaacacagttgggacccaaaacacaggaagtcagtcacgtttccttttcatttaatggggattccactatctcacactaatctgaaaggatgtggaagagcattagctggcgcatattaagcactttaagctccttgagtaaaaaggtggtatgtaatttatgcaaggtatttctccagttgggactcaggatattagttaatgagccatcactagaagaaaagcccattttcaactgctttgaaacttgcctggggtctgagcatgatgggaatagggagacagggtaggaaagggcgcctactcttcagggtctaaagatcaagtgggccttggatcgctaagctggctctgtttgatgctatttatgcaagttagggtctatgtattta

SEQ ID NO: 206 - DNA, 5084 bp, Homo sapiens
gatcccatcgcagctaccgcgatgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaaggtaacaacaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctgagcagaatcagtgtttgggtcacccctccaggaatgatctcttcttttggcttccatgatggttattttcttttctttcaacttgcatccaactccaggatagtgggcaccccactgcaatcctgtctttctgagcacactttagtggccgatgatttttgtcatcagccaccatcctattgcaaaggttccaactgtatatattcccaatagcaacgtagcttctaccatgaacagaaaacattctgatttggaaaaagagagggaggtatggactgggggccagagtcctttccaaggcttctccaattctgcccaaaaatatggttgatagtttacctgaataaatggtagtaatcacagttggccttcagaaccatccatagtagtatgatgatacaagattagaagctgaaaacctaagtcctttatgtggaaaacagaacatcattagaacaaaggacagagtatgaacacctgggcttaagaaatctagtatttcatgctgggaatgagacataggccatgaaaaaaatgatccccaagtgtgaacaaaagatgctcttctgtggaccactgcatgagcttttatactaccgacctggtttttaaatagagtttgctattagagcattgaattggagagaaggcctccctagccagcacttgtatatacgcatctataaattgtccgtgttcatacatttgaggggaaaacaccataaggtttcgtttctgtatacaaccctggcattatgtccactgtgtatagaagtagattaagagccatataagtttgaaggaaacagttaataccattttttaaggaaacaatataaccacaaagcacagtttgaacaaaatctcctcttttagctgatgaacttattctgtagattctgtggaacaagcctatcagcttcagaatggcattgtactcaatggatttgatgctgtttgacaaagttactgattcactgcatggctcccacaggagtgggaaaacactgccatcttagtttggattcttatgtagcaggaaataaagtataggtttagcctccttcgcaggcatgtcctggacaccgggccagtatctatatatgtgtatgtacgtttgtatgtgtgtagacaaatatttggaggggtatttttgccctgagtccaagagggtcctttagtacctgaaaagtaacttggctttcattattagtactgctcttgtttcttttcacatagctgtctagagtagcttaccagaagcttccatagtggtgcagaggaagtggaaggcatcagtccctatgtatttgcagttcacctgcacttaaggcactctgttatttagactcatcttactgtacctgttccttagaccttccataatgctactgtctcactgaaacatttaaattttaccctttagactgtagcctggatattattcttgtagtttacctctttaaaaacaaaacaaaacaaaacaaaaaactccccttcctcactgcccaatataaaaggcaaatgtgtacatggcagagtttgtgtgttgtcttgaaagattcaggtatgttgcctttatggtttcccccttctacatttcttagactacatttagagaactgtggccgttatctggaagtaaccatttgcactggagttctatgctctcgcacctttccaaagttaacagattttggggttgtgttgtcacccaagagattgttgtttgccatactttgtctgaaaaattcctttgtgtttctattgacttcaatgatagtaagaaaagtggttgttagttatagatgtctaggtacttcaggggcacttcattgagagttttgtcttgccatactttgtctgaaaaattcctttgtgtttctattgacttcaatgatagtaagaaaagtggttgttagttatagatgtctaggtacttcaggggcacttcattgagagttttgtcaatgtcttttgaatattcccaagcccatgagtccttgaaaatattttttatatatacagtaactttatgtgtaaatacataagcggcgtaagtttaaaggatgttggtgttccacgtgttttattcctgtatgttgtccaattgttgacagttctgaagaattc

SEQ ID NO: 207 - DNA, 3985 bp, Homo sapiens
gaagggcagacagagtgtccaaaagcgtgagagcacgaagtgaggagaaggtggagaagagagaagaggaagaggaagaggaagagaggaagcggagggaactgcggccaggctaaaaggggaagaagaggatcagcccaaggaggaggaagaggaaaacaagacaaacagccagtgcagaggagaggaacgtgtgtccagtgtcccgatccctgcggagctagtagctgagagctctgtgccctgggcaccttgcagccctgcacctgcctgccacttccccaccgaggccatgggcccaggagttctgctgctcctgctggtggccacagcttggcatggtcagggaatcccagtgatagagcccagtgtccctgagctggtcgtgaagccaggagcaacggtgaccttgcgatgtgtgggcaatggcagcgtggaatgggatggccccccatcacctcactggaccctgtactctgatggctccagcagcatcctcagcaccaacaacgctaccttccaaaacacggggacctatcgctgcactgagcctggagaccccctgggaggcagcgccgccatccacctctatgtcaaagaccctgcccggccctggaacgtgctagcacaggaggtggtcgtgttcgaggaccaggacgcactactgccctgtctgctcacagacccggtgctggaagcaggcgtctcgctggtgcgtgtgcgtggccggcccctcatgcgccacaccaactactccttctcgccctggcatggcttcaccatccacagggccaagttcattcagagccaggactatcaatgcagtgccctgatgggtggcaggaaggtgatgtccatcagcatccggctgaaagtgcagaaagtcatcccagggcccccagccttgacactggtgcctgcagagctggtgcggattcgaggggaggctgcccagatcgtgtgctcagccagcagcgttgatgttaactttgatgtcttcctccaacacaacaacaccaagctcgcaatccctcaacaatctgactttcataataaccgttaccaaaaagtcctgaccctcaacctcgatcaagtagatttccaacatgccggcaactactcctgcgtggccagcaacgtgcagggcaagcactccacctccatgttcttccgggtggtagagagtgcctacttgaacttgagctctgagcagaacctcatccaggaggtgaccgtgggggaggggctcaacctcaaagtcatggtggaggcctacccaggcctgcaaggttttaactggacctacctgggacccttttctgaccaccagcctgagcccaagcttgctaatgctaccaccaaggacacatacaggcacaccttcaccctctctctgccccgcctgaagccctctgaggctggccgctactccttcctggccagaaacccaggaggctggagagctctgacgtttgagctcacccttcgataccccccagaggtaagcgtcatatggacattcatcaacggctctggcacccttttgtgtgctgcctctgggtacccccagcccaacgtgacatggctgcagtgcagtggccacactgataggtgtgatgaggcccaagtgctgcaggtctgggatgacccataccctgaggtcctgagccaggagcccttccacaaggtgacggtgcagagcctgctgactgttgagaccttagagcacaaccaaacctacgagtgcagggcccacaacagcgtggggagtggctcctgggccttcatacccatctctgcaggagcccacacgcatcccccggatgagttcctcttcacaccagtggtggtcgcctgcatgtccatcatggccttgctgctgctgctgctcctgctgctattgtacaagtataagcagaagcccaagtaccaggtccgctggaagatcatcgagagctatgagggcaacagttatactttcatcgaccccacgcagctgccttacaacgagaagtgggagttcccccggaacaacctgcagtttggtaagaccctcggagctggagcctttgggaaggtggtggaggccacggcctttggtctgggcaaggaggatgctgtcctgaaggtggctgtgaagatgctgaagtccacggcccatgctgatgagaaggaggccctcatgtccgagctgaagatcatgagccacctgggccagcacgagaacatcgtcaaccttctgggagcctgtacccatggaggccctgtactggtcatcacggagtactgttgctatggcgacctgctcaactttctgcgaaggaaggctgaggccatgctgggacccagcctgagccccggccaggaccccgagggaggcgtcgactataagaacatccacctcgagaagaaatatgtccgcagggacagtggcttctccagccagggtgtggacacctatgtggagatgaggcctgtctccacttcttcaaatgactccttctctgagcaagacctggacaaggaggatggacggcccctggagctccgggacctgcttcacttctccagccaagtagcccagggcatggccttcctcgcttccaagaattgcatccaccgggacgtggcagcgcgtaacgtgctgttgaccaatggtcatgtggccaagattggggacttcgggctggctagggacatcatgaatgactccaactacattgtcaagggcaatgcccgcctgcctgtgaagtggatggccccagagagcatctttgactgtgtctacacggttcagagcgacgtctggtcctatggcatcctcctctgggagatcttctcacttgggctgaatccctaccctggcatcctggtgaacagcaagttctataaactggtgaaggatggataccaaatggcccagcctgcatttgccccaaagaatatatacagcatcatgcaggcctgctgggccttggagcccacccacagacccaccttccagcagatctgctccttccttcaggagcaggcccaagaggacaggagagagcgggactataccaatctgccgagcagcagcagaagcggtggcagcggcagcagcagcagtgagctggaggaggagagctctagtgagcacctgacctgctgcgagcaaggggatatcgcccagcccttgctgcagcccaacaactatcagttctgctgaggagttgacgacagggagtaccactctcccctcctccaaacttcaactcctccatggatggggcgacacggggagaacatacaaactctgccttcggtcatttcactcaacagctcggcccagctctgaaacttgggaaggtgagggattcaggggaggtcagaggatcccacttcctgagcatgggccatcactgccagtcaggggctgggggctgagccctcacccccccctcccctactgttctcatggtgttggcctcgtgtttgctatgccaactagtagaaccttctttcctaatccccttatcttcatggaaatggactgactttatgcctatgaagtccccaggagctacactgatactgagaaaaccaggctctttggggctagacagactggcagagagtgagatctccctctctgagaggagcagcagatgctcacagaccacactcagctcaggccccttggagcaggatggctcctctaagaatctcacaggacctcttagtctctgccctatacgccgccttcactccacagcctcacccctcccacccccatactggtactgctgtaatgagccaagtggcagctaaaagttgggggtgttctgcccagtcccgtcattctgggctagaaggcaggggaccttggcatgtggctggccacaccaagcaggaagcacaaactcccccaagctgactcatcctaactaacagtcacgccgtgggatgtctctgtccacattaaactaacagcattaatgca

SEQ ID NO: 208 - DNA, 3475 bp, Homo sapiens
cgaggcggcatccgagggctgggccggcgccctgggggaccccgggctccggaggccatgccggcgttggcgcgcgacgcgggcaccgtgccgctgctcgttgttttttctgcaatgatatttgggactattacaaatcaagatctgcctgtgatcaagtgtgttttaatcaatcataagaacaatgattcatcagtggggaagtcatcatcatatcccatggtatcagaatccccggaagacctcgggtgtgcgttgagaccccagagctcagggacagtgtacgaagctgccgctgtggaagtggatgtatctgcttccatcacactgcaagtgctggtcgatgccccagggaacatttcctgtctctgggtctttaagcacagctccctgaattgccagccacattttgatttacaaaacagaggagttgtttccatggtcattttgaaaatgacagaaacccaagctggagaatacctactttttattcagagtgaagctaccaattacacaatattgtttacagtgagtataagaaataccctgctttacacattaagaagaccttactttagaaaaatggaaaaccaggacgccctggtctgcatatctgagagcgttccagagccgatcgtggaatgggtgctttgcgattcacagggggaaagctgtaaagaagaaagtccagctgttgttaaaaaggaggaaaaagtgcttcatgaattatttgggacggacataaggtgctgtgccagaaatgaactgggcagggaatgcaccaggctgttcacaatagatctaaatcaaactcctcagaccacattgccacaattatttcttaaagtaggggaacccttatggataaggtgcaaagctgttcatgtgaaccatggattcgggctcacctgggaattagaaaacaaagcactcgaggagggcaactactttgagatgagtacctattcaacaaacagaactatgatacggattctgtttgcttttgtatcatcagtggcaagaaacgacaccggatactacacttgttcctcttcaaagcatcccagtcaatcagctttggttaccatcgtaggaaagggatttataaatgctaccaattcaagtgaagattatgaaattgaccaatatgaagagttttgtttttctgtcaggtttaaagcctacccacaaatcagatgtacgtggaccttctctcgaaaatcatttccttgtgagcaaaagggtcttgataacggatacagcatatccaagttttgcaatcataagcaccagccaggagaatatatattccatgcagaaaatgatgatgcccaatttaccaaaatgttcacgctgaatataagaaggaaacctcaagtgctcgcagaagcatcggcaagtcaggcgtcctgtttctcggatggatacccattaccatcttggacctggaagaagtgttcagacaagtctcccaactgcacagaagagatcacagaaggagtctggaatagaaaggctaacagaaaagtgtttggacagtgggtgtcgagcagtactctaaacatgagtgaagccataaaagggttcctggtcaagtgctgtgcatacaattcccttggcacatcttgtgagacgatccttttaaactctccaggccccttccctttcatccaagacaacatctcattctatgcaacaattggtgtttgtctcctcttcattgtcgttttaaccctgctaatttgtcacaagtacaaaaagcaatttaggtatgaaagccagctacagatggtacaggtgaccggctcctcagataatgagtacttctacgttgatttcagagaatatgaatatgatctcaaatgggagtttccaagagaaaatttagagtttgggaaggtactaggatcaggtgcttttggaaaagtgatgaacgcaacagcttatggaattagcaaaacaggagtctcaatccaggttgccgtcaaaatgctgaaagaaaaagcagacagctctgaaagagaggcactcatgtcagaactcaagatgatgacccagctgggaagccacgagaatattgtgaacctgctgggggcgtgcacactgtcaggaccaatttacttgatttttgaatactgttgctatggtgatcttctcaactatctaagaagtaaaagagaaaaatttcacaggacttggacagagattttcaaggaacacaatttcagtttttaccccactttccaatcacatccaaattccagcatgcctggttcaagagaagttcagatacacccggactcggatcaaatctcagggcttcatgggaattcatttcactctgaagatgaaattgaatatgaaaaccaaaaaaggctggaagaagaggaggacttgaatgtgcttacatttgaagatcttctttgctttgcatatcaagttgccaaaggaatggaatttctggaatttaagtcgtgtgttcacagagacctggccgccaggaacgtgcttgtcacccacgggaaagtggtgaagatatgtgactttggattggctcgagatatcatgagtgattccaactatgttgtcaggggcaatgcccgtctgcctgtaaaatggatggcccccgaaagcctgtttgaaggcatctacaccattaagagtgatgtctggtcatatggaatattactgtgggaaatcttctcacttggtgtgaatccttaccctggcattccggttgatgctaacttctacaaactgattcaaaatggatttaaaatggatcagccattttatgctacagaagaaatatacattataatgcaatcctgctgggcttttgactcaaggaaacggccatccttccctaatttgacttcgtttttaggatgtcagctggcagatgcagaagaagcgatgtatcagaatgtggatggccgtgtttcggaatgtcctcacacctaccaaaacaggcgacctttcagcagagagatggatttggggctactctctccgcaggctcaggtcgaagattcgtagaggaacaatttagttttaaggacttcatccctccacctatccctaacaggctgtagattaccaaaacaagattaatttcatcactaaaagaaaatctattatcaactgctgcttcaccagacttttctctagaagccgtctgcgtttactcttgttttcaaagggacttttgtaaaatcaaatcatcctgtcacaaggcaggaggagctgataatgaactttattggagcattgatctgcatccaaggccttctcaggccggcttgagtgaattgtgtacctgaagtacagtatattcttgtaaatacataaaacaaaagcattttgctaaggagaagctaatatgattttttaagtctatgttttaaaataatatgtaaatttttcagctatttagtgatatattttatgggtgggaataaaatttctactacag

SEQ ID NO: 209 - DNA, 4776 bp, Homo sapiens
cccacgcgcagcggccggagatgcagcggggcgccgcgctgtgcctgcgactgtggctctgcctgggactcctggacggcctggtgagtgactactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctctatgacatccagctgttgcccaggaagtcgctggagctgctggtaggggagaagctggtcctcaactgcaccgtgtgggctgagtttaactcaggtgtcacctttgactgggactacccagggaagcaggcagagcggggtaagtgggtgcccgagcgacgctcccaacagacccacacagaactctccagcatcctgaccatccacaacgtcagccagcacgacctgggctcgtatgtgtgcaaggccaacaacggcatccagcgatttcgggagagcaccgaggtcattgtgcatgaaaatcccttcatcagcgtcgagtggctcaaaggacccatcctggaggccacggcaggagacgagctggtgaagctgcccgtgaagctggcagcgtaccccccgcccgagttccagtggtacaaggatggaaaggcactgtccgggcgccacagtccacatgccctggtgctcaaggaggtgacagaggccagcacaggcacctacaccctcgccctgtggaactccgctgctggcctgaggcgcaacatcagcctggagctggtggtgaatgtgcccccccagatacatgagaaggaggcctcctcccccagcatctactcgcgtcacagccgccaggccctcacctgcacggcctacggggtgcccctgcctctcagcatccagtggcactggcggccctggacaccctgcaagatgtttgcccagcgtagtctccggcggcggcagcagcaagacctcatgccacagtgccgtgactggagggcggtgaccacgcaggatgccgtgaaccccatcgagagcctggacacctggaccgagtttgtggagggaaagaataagactgtgagcaagctggtgatccagaatgccaacgtgtctgccatgtacaagtgtgtggtctccaacaaggtgggccaggatgagcggctcatctacttctatgtgaccaccatccccgacggcttcaccatcgaatccaagccatccgaggagctactagagggccagccggtgctcctgagctgccaagccgacagctacaagtacgagcatctgcgctggtaccgcctcaacctgtccacgctgcacgatgcgcacgggaacccgcttctgctcgactgcaagaacgtgcatctgttcgccacccctctggccgccagcctggaggaggtggcacctggggcgcgccacgccacgctcagcctgagtatcccccgcgtcgcgcccgagcacgagggccactatgtgtgcgaagtgcaagaccggcgcagccatgacaagcactgccacaagaagtacctgtcggtgcaggccctggaagcccctcggctcacgcagaacttgaccgacctcctggtgaacgtgagcgactcgctggagatgcagtgcttggtggccggagcgcacgcgcccagcatcgtgtggtacaaagacgagaggctgctggaggaaaagtctggagtcgacttggcggactccaaccagaagctgagcatccagcgcgtgcgcgaggaggatgcgggaccgtatctgtgcagcgtgtgcagacccaagggctgcgtcaactcctccgccagcgtggccgtggaaggctccgaggataagggcagcatggagatcgtgatccttgtcggtaccggcgtcatcgctgtcttcttctgggtcctcctcctcctcatcttctgtaacatgaggaggccggcccacgcagacatcaagacgggctacctgtccatcatcatggaccccggggaggtgcctctggaggagcaatgcgaatacctgtcctacgatgccagccagtgggaattcccccgagagcggctgcacctggggagagtgctcggctacggcgccttcgggaaggtggtggaagcctccgctttcggcatccacaagggcagcagctgtgacaccgtggccgtgaaaatgctgaaagagggcgccacggccagcgagcagcgcgcgctgatgtcggagctcaagatcctcattcacatcggcaaccacctcaacgtggtcaacctcctcggggcgtgcaccaagccgcagggccccctcatggtgatcgtggagttctgcaagtacggcaacctctccaacttcctgcgcgccaagcgggacgccttcagcccctgcgcggagaagtctcccgagcagcgcggacgcttccgcgccatggtggagctcgccaggctggatcggaggcggccggggagcagcgacagggtcctcttcgcgcggttctcgaagaccgagggcggagcgaggcgggcttctccagaccaagaagctgaggacctgtggctgagcccgctgaccatggaagatcttgtctgctacagcttccaggtggccagagggatggagttcctggcttcccgaaagtgcatccacagagacctggctgctcggaacattctgctgtcggaaagcgacgtggtgaagatctgtgactttggccttgcccgggacatctacaaagaccccgactacgtccgcaagggcagtgcccggctgcccctgaagtggatggcccctgaaagcatcttcgacaaggtgtacaccacgcagagtgacgtgtggtcctttggggtgcttctctgggagatcttctctctgggggcctccccgtaccctggggtgcagatcaatgaggagttctgccagcgcgtgagagacggcacaaggatgagggccccggagctggccactcccgccatacgccacatcatgctgaactgctggtccggagaccccaaggcgagacctgcattctcggagctggtggagatcctgggggacctgctccagggcaggggcctgcaagaggaagaggaggtctgcatggccccgcgcagctctcagagctcagaagagggcagcttctcgcaggtgtccaccatggccctacacatcgcccaggctgacgctgaggacagcccgccaagcctgcagcgccacagcctggccgccaggtattacaactgggtgtcctttcccgggtgcctggccagaggggctgagacccgtggttcctccaggatgaagacatttgaggaattccccatgaccccaacgacctacaaaggctctgtggacaaccagacagacagtgggatggtgctggcctcggaggagtttgagcagatagagagcaggcatagacaagaaagcggcttcagctgtaaaggacctggccagaatgtggctgtgaccagggcacaccctgactcccaagggaggcggcggcggcctgagcggggggcccgaggaggccaggtgttttacaacagcgagtatggggagctgtcggagccaagcgaggaggaccactgctccccgtctgcccgcgtgactttcttcacagacaacagctactaagcagcatcggacaagacccccagcacttgggggttcaggcccggcagggcgggcagagggctggaggcccaggctgggaactcatctggttgaactctggtggcacaggagtgtcctcttccctctctgcagacttcccagctaggaagagcaggactccaggcccaaggctcccggaattccgtcaccacgactggccagggcacgctccagctgccccggcccctccccctgagattcagatgtcatttagttcagcatccgcaggtgctggtcccggggccagcacttccatgggaatgtctctttggcgacctcctttcatcacactgggtggtggcctggtccctgttttcccacgaggaatctgtgggtctgggagtcacacagtgttggaggttaaggcatacgagagcagaggtctcccaaacgccctttcctcctcaggcacacagctactctccccacgagggctggctggcctcacccacccctgcacagttgaagggaggggctgtgtttccatctcaaagaaggcatttgcagggtcctcttctgggcctgaccaaacagccaactagcccctggggtggccaccagtatgacagtattatacgctggcaacacagaggcagcccgcacacctgcgagtggcaaactgtcc

SEQ ID NO: 210 - DNA, 4450 bp, Homo sapiens
acccacgcgcagcggccggagatgcagcggggcgccgcgctgtgcctgcgactgtggctctgcctgggactcctggacggcctggtgagtgactactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctctatgacatccagctgttgcccaggaagtcgctggagctgctggtaggggagaagctggtcctcaactgcaccgtgtgggctgagtttaactcaggtgtcacctttgactgggactacccagggaagcaggcagagcggggtaagtgggtgcccgagcgacgctcccaacagacccacacagaactctccagcatcctgaccatccacaacgtcagccagcacgacctgggctcgtatgtgtgcaaggccaacaacggcatccagcgatttcgggagagcaccgaggtcattgtgcatgaaaatcccttcatcagcgtcgagtggctcaaaggacccatcctggaggccacggcaggagacgagctggtgaagctgcccgtgaagctggcagcgtaccccccgcccgagttccagtggtacaaggatggaaaggcactgtccgggcgccacagtccacatgccctggtgctcaaggaggtgacagaggccagcacaggcacctacaccctcgccctgtggaactccgctgctggcctgaggcgcaacatcagcctggagctggtggtgaatgtgcccccccagatacatgagaaggaggcctcctcccccagcatctactcgcgtcacagccgccaggccctcacctgcacggcctacggggtgcccctgcctctcagcatccagtggcactggcggccctggacaccctgcaagatgtttgcccagcgtagtctccggcggcggcagcagcaagacctcatgccacagtgccgtgactggagggcggtgaccacgcaggatgccgtgaaccccatcgagagcctggacacctggaccgagtttgtggagggaaagaataagactgtgagcaagctggtgatccagaatgccaacgtgtctgccatgtacaagtgtgtggtctccaacaaggtgggccaggatgagcggctcatctacttctatgtgaccaccatccccgacggcttcaccatcgaatccaagccatccgaggagctactagagggccagccggtgctcctgagctgccaagccgacagctacaagtacgagcatctgcgctggtaccgcctcaacctgtccacgctgcacgatgcgcacgggaacccgcttctgctcgactgcaagaacgtgcatctgttcgccacccctctggccgccagcctggaggaggtggcacctggggcgcgccacgccacgctcagcctgagtatcccccgcgtcgcgcccgagcacgagggccactatgtgtgcgaagtgcaagaccggcgcagccatgacaagcactgccacaagaagtacctgtcggtgcaggccctggaagcccctcggctcacgcagaacttgaccgacctcctggtgaacgtgagcgactcgctggagatgcagtgcttggtggccggagcgcacgcgcccagcatcgtgtggtacaaagacgagaggctgctggaggaaaagtctggagtcgacttggcggactccaaccagaagctgagcatccagcgcgtgcgcgaggaggatgcgggaccgtatctgtgcagcgtgtgcagacccaagggctgcgtcaactcctccgccagcgtggccgtggaaggctccgaggataagggcagcatggagatcgtgatccttgtcggtaccggcgtcatcgctgtcttcttctgggtcctcctcctcctcatcttctgtaacatgaggaggccggcccacgcagacatcaagacgggctacctgtccatcatcatggaccccggggaggtgcctctggaggagcaatgcgaatacctgtcctacgatgccagccagtgggaattcccccgagagcggctgcacctggggagagtgctcggctacggcgccttcgggaaggtggtggaagcctccgctttcggcatccacaagggcagcagctgtgacaccgtggccgtgaaaatgctgaaagagggcgccacggccagcgagcagcgcgcgctgatgtcggagctcaagatcctcattcacatcggcaaccacctcaacgtggtcaacctcctcggggcgtgcaccaagccgcagggccccctcatggtgatcgtggagttctgcaagtacggcaacctctccaacttcctgcgcgccaagcgggacgccttcagcccctgcgcggagaagtctcccgagcagcgcggacgcttccgcgccatggtggagctcgccaggctggatcggaggcggccggggagcagcgacagggtcctcttcgcgcggttctcgaagaccgagggcggagcgaggcgggcttctccagaccaagaagctgaggacctgtggctgagcccgctgaccatggaagatcttgtctgctacagcttccaggtggccagagggatggagttcctggcttcccgaaagtgcatccacagagacctggctgctcggaacattctgctgtcggaaagcgacgtggtgaagatctgtgactttggccttgcccgggacatctacaaagaccccgactacgtccgcaagggcagtgcccggctgcccctgaagtggatggcccctgaaagcatcttcgacaaggtgtacaccacgcagagtgacgtgtggtcctttggggtgcttctctgggagatcttctctctgggggcctccccgtaccctggggtgcagatcaatgaggagttctgccagcgcgtgagagacggcacaaggatgagggccccggagctggccactcccgccatacgccacatcatgctgaactgctggtccggagaccccaaggcgagacctgcattctcggacctggtggagatcctgggggacctgctccagggcaggggcctgcaagaggaagaggaggtctgcatggccccgcgcagctctcagagctcagaagagggcagcttctcgcaggtgtccaccatggccctacacatcgcccaggctgacgctgaggacagcccgccaagcctgcagcgccacagcctggccgccaggtattacaactgggtgtcctttcccgggtgcctggccagaggggctgagacccgtggttcctccaggatgaagacatttgaggaattccccatgaccccaacgacctacaaaggctctgtggacaaccagacagacagtgggatggtgctggcctcggaggagtttgagcagatagagagcaggcatagacaagaaagcggcttcaggtagctgaagcagagagagagaaggcagcatacgtcagcattttcttctctgcacttataagaaagatcaaagactttaagactttcgctatttcttctactgctatctactacaaacttcaaagaggaaccaggaggacaagaggagcatgaaagtggacaaggagtgtgaccactgaagcaccacagggaggggttaggcctccggatgactgcgggcaggcctggataatatccagcctcccacaagaagctggtggagcagagtgttccctgactcctccaaggaaagggagacgccctttcatggtctgctgagtaacaggtgccttcccagacactggcgttactgcttgaccaaagagccctcaagcggcccttatgccagcgtgacagagggctcacctcttgccttctaggtcacttctcacaatgtcccttcagcacctgaccctgtgcccgccgattattccttggtaatatgagtaatacatcaaagagtagtattaaaagctaattaatcatgtttataaaaa

SEQ ID NO: 211 - DNA, 19 bp, Artificial
  Target Sequence
tgcctgaaatggtgagtaa

SEQ ID NO: 212 - DNA, 19 bp, Artificial
  Target Sequence
aaaggctgagcataactaa

SEQ ID NO: 213 - DNA, 19 bp, Artificial
  Target Sequence
ctagctgtacctacttcaa

SEQ ID NO: 214 - DNA, 19 bp, Artificial
  Target Sequence
gacctttcgtagagatgta

SEQ ID NO: 215 - DNA, 19 bp, Artificial
  Target Sequence
ctttatacttgtcgtgtaa

SEQ ID NO: 216 - DNA, 19 bp, Artificial
  Target Sequence
ccatcattcaaatctgtta

SEQ ID NO: 217 - DNA, 19 bp, Artificial
  Target Sequence
taacacctcagtgcatata

SEQ ID NO: 218 - DNA, 19 bp, Artificial
  Target Sequence
cttaccggctctctatgaa

SEQ ID NO: 219 - DNA, 19 bp, Artificial
  Target Sequence
ttaccggctctctatgaaa

SEQ ID NO: 220 - DNA, 19 bp, Artificial
  Target Sequence
cggaagttgtatggttaaa

SEQ ID NO: 221 - DNA, 19 bp, Artificial
  Target Sequence
actcgtggctactcgttaa

SEQ ID NO: 222 - DNA, 19 bp, Artificial
  Target Sequence
caatcttgctgagcataaa

SEQ ID NO: 223 - DNA, 19 bp, Artificial
  Target Sequence
aaacctcactgccactcta

SEQ ID NO: 224 - DNA, 19 bp, Artificial
  Target Sequence
ctcagcgcatggcaataat

SEQ ID NO: 225 - DNA, 19 bp, Artificial
  Target Sequence
tcagcgcatggcaataata

SEQ ID NO: 226 - DNA, 19 bp, Artificial
  Target Sequence
acaatgcactacagtatta

SEQ ID NO: 227 - DNA, 19 bp, Artificial
  Target Sequence
agcatacctcactgttcaa

SEQ ID NO: 228 - DNA, 19 bp, Artificial
  Target Sequence
ctcttctggctcctattaa

SEQ ID NO: 229 - DNA, 19 bp, Artificial
  Target Sequence
actgactacctatcaatta

SEQ ID NO: 230 - DNA, 19 bp, Artificial
  Target Sequence
ctgactacctatcaattat

SEQ ID NO: 231 - DNA, 19 bp, Artificial
  Target Sequence
gcatcagcatttggcatta

SEQ ID NO: 232 - DNA, 19 bp, Artificial
  Target Sequence
catcagcatttggcattaa

SEQ ID NO: 233 - DNA, 19 bp, Artificial
  Target Sequence
tgatggtgattgttgaata

SEQ ID NO: 234 - DNA, 19 bp, Artificial
  Target Sequence
atggaaatctctccaacta

SEQ ID NO: 235 - DNA, 19 bp, Artificial
  Target Sequence
ccaactacctcaagagcaa

SEQ ID NO: 236 - DNA, 19 bp, Artificial
  Target Sequence
cgaatctatctttgacaaa

SEQ ID NO: 237 - DNA, 19 bp, Artificial
  Target Sequence
tgagtactctactcctgaa

SEQ ID NO: 238 - DNA, 19 bp, Artificial
  Target Sequence
gagtactctactcctgaaa

SEQ ID NO: 239 - DNA, 19 bp, Artificial
  Target Sequence
gccatactgacaggaaata

SEQ ID NO: 240 - DNA, 19 bp, Artificial
  Target Sequence
acttgagagtaaccagtaa

SEQ ID NO: 241 - DNA, 19 bp, Artificial
  Target Sequence
cttgagagtaaccagtaaa

SEQ ID NO: 242 - DNA, 19 bp, Artificial
  Target Sequence
acaactcggtggtcctgta

SEQ ID NO: 243 - DNA, 19 bp, Artificial
  Target Sequence
tgaagaacactactgctaa

SEQ ID NO: 244 - DNA, 19 bp, Artificial
  Target Sequence
ttactcagtgttagagaaa

SEQ ID NO: 245 - DNA, 19 bp, Artificial
  Target Sequence
gcaggaccagtttgattga

SEQ ID NO: 246 - DNA, 19 bp, Artificial
  Target Sequence
tcctctagcaggcctaaga

SEQ ID NO: 247 - DNA, 19 bp, Artificial
  Target Sequence
ctagtaagatgcactgaaa

SEQ ID NO: 248 - DNA, 19 bp, Artificial
  Target Sequence
tgatggccttacactgaaa

SEQ ID NO: 249 - DNA, 19 bp, Artificial
  Target Sequence
ccaaaccaattcaccaaca

SEQ ID NO: 250 - DNA, 19 bp, Artificial
  Target Sequence
agcattagctggcgcatat

SEQ ID NO: 251 - DNA, 19 bp, Artificial
  Target Sequence
cattagctggcgcatatta

SEQ ID NO: 252 - DNA, 19 bp, Artificial
  Target Sequence
attagctggcgcatattaa

SEQ ID NO: 253 - DNA, 19 bp, Artificial
  Target Sequence
gcgcatattaagcacttta

SEQ ID NO: 254 - DNA, 19 bp, Artificial
  Target Sequence
cgcatattaagcactttaa

SEQ ID NO: 255 - DNA, 19 bp, Artificial
  Target Sequence
ggactcaggatattagtta

SEQ ID NO: 256 - DNA, 19 bp, Artificial
  Target Sequence
gactcaggatattagttaa

SEQ ID NO: 257 - DNA, 19 bp, Artificial
  Target Sequence
ctaagctggctctgtttga

SEQ ID NO: 258 - DNA, 19 bp, Artificial
  Target Sequence
cgacgagattaggctgtta

SEQ ID NO: 259 - DNA, 19 bp, Artificial
  Target Sequence
aaacacggcttaagcaatt

SEQ ID NO: 260 - DNA, 19 bp, Artificial
  Target Sequence
cacagtgacgtgcacaata

SEQ ID NO: 261 - DNA, 19 bp, Artificial
  Target Sequence
acagtgacgtgcacaataa

SEQ ID NO: 262 - DNA, 19 bp, Artificial
  Target Sequence
cagtgacgtgcacaataaa

SEQ ID NO: 263 - DNA, 19 bp, Artificial
  Target Sequence
agactaaactacaggagaa

SEQ ID NO: 264 - DNA, 19 bp, Artificial
  Target Sequence
acggtgacttcaattatga

SEQ ID NO: 265 - DNA, 19 bp, Artificial
  Target Sequence
cagttcagcgagagttaat

SEQ ID NO: 266 - DNA, 19 bp, Artificial
  Target Sequence
agcagtggatctatatgaa

SEQ ID NO: 267 - DNA, 19 bp, Artificial
  Target Sequence
aacagaaccttcactgata

SEQ ID NO: 268 - DNA, 19 bp, Artificial
  Target Sequence
acagaaccttcactgataa

SEQ ID NO: 269 - DNA, 19 bp, Artificial
  Target Sequence
cagaaccttcactgataaa

SEQ ID NO: 270 - DNA, 19 bp, Artificial
  Target Sequence
cttcatctaacgagattaa

SEQ ID NO: 271 - DNA, 19 bp, Artificial
  Target Sequence
gtccaattctgacgtcaat

SEQ ID NO: 272 - DNA, 19 bp, Artificial
  Target Sequence
ctagtggttcagagttcta

SEQ ID NO: 273 - DNA, 19 bp, Artificial
  Target Sequence
atggcacggttgaatgtaa

SEQ ID NO: 274 - DNA, 19 bp, Artificial
  Target Sequence
ctggcatgatgtgcattat

SEQ ID NO: 275 - DNA, 19 bp, Artificial
  Target Sequence
ttgtgatgattctgaccta

SEQ ID NO: 276 - DNA, 19 bp, Artificial
  Target Sequence
tgatgattctgacctacaa

SEQ ID NO: 277 - DNA, 19 bp, Artificial
  Target Sequence
aggttgttgaggagataaa

SEQ ID NO: 278 - DNA, 19 bp, Artificial
  Target Sequence
acttccttatgatcacaaa

SEQ ID NO: 279 - DNA, 19 bp, Artificial
  Target Sequence
gcaactgcttatggcttaa

SEQ ID NO: 280 - DNA, 19 bp, Artificial
  Target Sequence
aactgcttatggcttaatt

SEQ ID NO: 281 - DNA, 19 bp, Artificial
  Target Sequence
ctcatggtcggatcacaaa

SEQ ID NO: 282 - DNA, 19 bp, Artificial
  Target Sequence
catggtcggatcacaaaga

SEQ ID NO: 283 - DNA, 19 bp, Artificial
  Target Sequence
taaaggaaacgctcgacta

SEQ ID NO: 284 - DNA, 19 bp, Artificial
  Target Sequence
tggaatgccggtcgattct

SEQ ID NO: 285 - DNA, 19 bp, Artificial
  Target Sequence
cggtcgattctaagttcta

SEQ ID NO: 286 - DNA, 19 bp, Artificial
  Target Sequence
tcgattctaagttctacaa

SEQ ID NO: 287 - DNA, 19 bp, Artificial
  Target Sequence
gaccattctgtgcggatca

SEQ ID NO: 288 - DNA, 19 bp, Artificial
  Target Sequence
aaaggttccaactgtatat

SEQ ID NO: 289 - DNA, 19 bp, Artificial
  Target Sequence
aaggttccaactgtatata

SEQ ID NO: 290 - DNA, 19 bp, Artificial
  Target Sequence
gttgatagtttacctgaat

SEQ ID NO: 291 - DNA, 19 bp, Artificial
  Target Sequence
ccatagtagtatgatgata

SEQ ID NO: 292 - DNA, 19 bp, Artificial
  Target Sequence
ctaagtcctttatgtggaa

SEQ ID NO: 293 - DNA, 19 bp, Artificial
  Target Sequence
tgagacataggccatgaaa

SEQ ID NO: 294 - DNA, 19 bp, Artificial
  Target Sequence
acttgtatatacgcatcta

SEQ ID NO: 295 - DNA, 19 bp, Artificial
  Target Sequence
accataaggtttcgtttct

SEQ ID NO: 296 - DNA, 19 bp, Artificial
  Target Sequence
gtagattaagagccatata

SEQ ID NO: 297 - DNA, 19 bp, Artificial
  Target Sequence
tgtagattctgtggaacaa

SEQ ID NO: 298 - DNA, 19 bp, Artificial
  Target Sequence
cttatgtagcaggaaataa

SEQ ID NO: 299 - DNA, 19 bp, Artificial
  Target Sequence
agtaacttggctttcatta

SEQ ID NO: 300 - DNA, 19 bp, Artificial
  Target Sequence
ccatagtggtgcagaggaa

SEQ ID NO: 301 - DNA, 19 bp, Artificial
  Target Sequence
ttccttagaccttccataa

SEQ ID NO: 302 - DNA, 19 bp, Artificial
  Target Sequence
tccttagaccttccataat

SEQ ID NO: 303 - DNA, 19 bp, Artificial
  Target Sequence
gactgtagcctggatatta

SEQ ID NO: 304 - DNA, 19 bp, Artificial
  Target Sequence
tcaggtatgttgcctttat

SEQ ID NO: 305 - DNA, 19 bp, Artificial
  Target Sequence
agagaactgtggccgttat

SEQ ID NO: 306 - DNA, 19 bp, Artificial
  Target Sequence
taagcggcgtaagtttaaa

SEQ ID NO: 307 - DNA, 19 bp, Artificial
  Target Sequence
ccagcagcgttgatgttaa

SEQ ID NO: 308 - DNA, 19 bp, Artificial
  Target Sequence
cctcaacctcgatcaagta

SEQ ID NO: 309 - DNA, 19 bp, Artificial
  Target Sequence
tcaacctcgatcaagtaga

SEQ ID NO: 310 - DNA, 19 bp, Artificial
  Target Sequence
caacctcgatcaagtagat

SEQ ID NO: 311 - DNA, 19 bp, Artificial
  Target Sequence
tccaacatgccggcaacta

SEQ ID NO: 312 - DNA, 19 bp, Artificial
  Target Sequence
tagagagtgcctacttgaa

SEQ ID NO: 313 - DNA, 19 bp, Artificial
  Target Sequence
ggagagctctgacgtttga

SEQ ID NO: 314 - DNA, 19 bp, Artificial
  Target Sequence
agcgtcatatggacattca

SEQ ID NO: 315 - DNA, 19 bp, Artificial
  Target Sequence
tcatatggacattcatcaa

SEQ ID NO: 316 - DNA, 19 bp, Artificial
  Target Sequence
gctgactgttgagacctta

SEQ ID NO: 317 - DNA, 19 bp, Artificial
  Target Sequence
tcctgctgctattgtacaa

SEQ ID NO: 318 - DNA, 19 bp, Artificial
  Target Sequence
tgctgctattgtacaagta

SEQ ID NO: 319 - DNA, 19 bp, Artificial
  Target Sequence
ctgctattgtacaagtata

SEQ ID NO: 320 - DNA, 19 bp, Artificial
  Target Sequence
agatcatcgagagctatga

SEQ ID NO: 321 - DNA, 19 bp, Artificial
  Target Sequence
gctatggcgacctgctcaa

SEQ ID NO: 322 - DNA, 19 bp, Artificial
  Target Sequence
ctgctcaactttctgcgaa

SEQ ID NO: 323 - DNA, 19 bp, Artificial
  Target Sequence
ggaggcgtcgactataaga

SEQ ID NO: 324 - DNA, 19 bp, Artificial
  Target Sequence
ataagaacatccacctcga

SEQ ID NO: 325 - DNA, 19 bp, Artificial
  Target Sequence
agaacatccacctcgagaa

SEQ ID NO: 326 - DNA, 19 bp, Artificial
  Target Sequence
gccttcctcgcttccaaga

SEQ ID NO: 327 - DNA, 19 bp, Artificial
  Target Sequence
gtaacgtgctgttgaccaa

SEQ ID NO: 328 - DNA, 19 bp, Artificial
  Target Sequence
gaacagcaagttctataaa

SEQ ID NO: 329 - DNA, 19 bp, Artificial
  Target Sequence
agttctataaactggtgaa

SEQ ID NO: 330 - DNA, 19 bp, Artificial
  Target Sequence
cttcggtcatttcactcaa

SEQ ID NO: 331 - DNA, 19 bp, Artificial
  Target Sequence
tcggtcatttcactcaaca

SEQ ID NO: 332 - DNA, 19 bp, Artificial
  Target Sequence
cctcgtgtttgctatgcca

SEQ ID NO: 333 - DNA, 19 bp, Artificial
  Target Sequence
tttgctatgccaactagta

SEQ ID NO: 334 - DNA, 19 bp, Artificial
  Target Sequence
caactagtagaaccttctt

SEQ ID NO: 335 - DNA, 19 bp, Artificial
  Target Sequence
gtagaaccttctttcctaa

SEQ ID NO: 336 - DNA, 19 bp, Artificial
  Target Sequence
agaaccttctttcctaatc

SEQ ID NO: 337 - DNA, 19 bp, Artificial
  Target Sequence
tggaaatggactgacttta

SEQ ID NO: 338 - DNA, 19 bp, Artificial
  Target Sequence
tggactgactttatgccta

SEQ ID NO: 339 - DNA, 19 bp, Artificial
  Target Sequence
ggactgactttatgcctat

SEQ ID NO: 340 - DNA, 19 bp, Artificial
  Target Sequence
actgactttatgcctatga

SEQ ID NO: 341 - DNA, 19 bp, Artificial
  Target Sequence
ctgactttatgcctatgaa

SEQ ID NO: 342 - DNA, 19 bp, Artificial
  Target Sequence
caggatggctcctctaaga

SEQ ID NO: 343 - DNA, 19 bp, Artificial
  Target Sequence
ggatggctcctctaagaat

SEQ ID NO: 344 - DNA, 19 bp, Artificial
  Target Sequence
catactggtactgctgtaa

SEQ ID NO: 345 - DNA, 19 bp, Artificial
  Target Sequence
gagccaagtggcagctaaa

SEQ ID NO: 346 - DNA, 19 bp, Artificial
  Target Sequence
agctgactcatcctaacta

SEQ ID NO: 347 - DNA, 19 bp, Artificial
  Target Sequence
gctgactcatcctaactaa

SEQ ID NO: 348 - DNA, 19 bp, Artificial
  Target Sequence
agagtgaagctaccaatta

SEQ ID NO: 349 - DNA, 19 bp, Artificial
  Target Sequence
aaagtccagctgttgttaa

SEQ ID NO: 350 - DNA, 19 bp, Artificial
  Target Sequence
aagtccagctgttgttaaa

SEQ ID NO: 351 - DNA, 19 bp, Artificial
  Target Sequence
cagaccacattgccacaat

SEQ ID NO: 352 - DNA, 19 bp, Artificial
  Target Sequence
gaccacattgccacaatta

SEQ ID NO: 353 - DNA, 19 bp, Artificial
  Target Sequence
accacattgccacaattat

SEQ ID NO: 354 - DNA, 19 bp, Artificial
  Target Sequence
tggttaccatcgtaggaaa

SEQ ID NO: 355 - DNA, 19 bp, Artificial
  Target Sequence
ccaattcaagtgaagatta

SEQ ID NO: 356 - DNA, 19 bp, Artificial
  Target Sequence
gataacggatacagcatat

SEQ ID NO: 357 - DNA, 19 bp, Artificial
  Target Sequence
gtcaagtgctgtgcataca

SEQ ID NO: 358 - DNA, 19 bp, Artificial
  Target Sequence
tgctaatttgtcacaagta

SEQ ID NO: 359 - DNA, 19 bp, Artificial
  Target Sequence
gtgaccggctcctcagata

SEQ ID NO: 360 - DNA, 19 bp, Artificial
  Target Sequence
tgaccggctcctcagataa

SEQ ID NO: 361 - DNA, 19 bp, Artificial
  Target Sequence
ctacgttgatttcagagaa

SEQ ID NO: 362 - DNA, 19 bp, Artificial
  Target Sequence
tacgttgatttcagagaat

SEQ ID NO: 363 - DNA, 19 bp, Artificial
  Target Sequence
acgttgatttcagagaata

SEQ ID NO: 364 - DNA, 19 bp, Artificial
  Target Sequence
caatccaggttgccgtcaa

SEQ ID NO: 365 - DNA, 19 bp, Artificial
  Target Sequence
aatccaggttgccgtcaaa

SEQ ID NO: 366 - DNA, 19 bp, Artificial
  Target Sequence
tgatcttctcaactatcta

SEQ ID NO: 367 - DNA, 19 bp, Artificial
  Target Sequence
caagagaagttcagataca

SEQ ID NO: 368 - DNA, 19 bp, Artificial
  Target Sequence
ggacttgaatgtgcttaca

SEQ ID NO: 369 - DNA, 19 bp, Artificial
  Target Sequence
tggattggctcgagatatc

SEQ ID NO: 370 - DNA, 19 bp, Artificial
  Target Sequence
catgagtgattccaactat

SEQ ID NO: 371 - DNA, 19 bp, Artificial
  Target Sequence
gaaggcatctacaccatta

SEQ ID NO: 372 - DNA, 19 bp, Artificial
  Target Sequence
cggttgatgctaacttcta

SEQ ID NO: 373 - DNA, 19 bp, Artificial
  Target Sequence
atgcagaagaagcgatgta

SEQ ID NO: 374 - DNA, 19 bp, Artificial
  Target Sequence
gtttcggaatgtcctcaca

SEQ ID NO: 375 - DNA, 19 bp, Artificial
  Target Sequence
gaagattcgtagaggaaca

SEQ ID NO: 376 - DNA, 19 bp, Artificial
  Target Sequence
cctaacaggctgtagatta

SEQ ID NO: 377 - DNA, 19 bp, Artificial
  Target Sequence
ctaacaggctgtagattac

SEQ ID NO: 378 - DNA, 19 bp, Artificial
  Target Sequence
acaggctgtagattaccaa

SEQ ID NO: 379 - DNA, 19 bp, Artificial
  Target Sequence
tagaagccgtctgcgttta

SEQ ID NO: 380 - DNA, 19 bp, Artificial
  Target Sequence
agaagccgtctgcgtttac

SEQ ID NO: 381 - DNA, 19 bp, Artificial
  Target Sequence
gaagccgtctgcgtttact

SEQ ID NO: 382 - DNA, 19 bp, Artificial
  Target Sequence
tctgcgtttactcttgttt

SEQ ID NO: 383 - DNA, 19 bp, Artificial
  Target Sequence
cggcttgagtgaattgtgt

SEQ ID NO: 384 - DNA, 19 bp, Artificial
  Target Sequence
gaattgtgtacctgaagta

SEQ ID NO: 385 - DNA, 19 bp, Artificial
  Target Sequence
gtacctgaagtacagtata

SEQ ID NO: 386 - DNA, 19 bp, Artificial
  Target Sequence
tacctgaagtacagtatat

SEQ ID NO: 387 - DNA, 19 bp, Artificial
  Target Sequence
tgctaaggagaagctaata

SEQ ID NO: 388 - DNA, 19 bp, Artificial
  Target Sequence
ggagaagctaatatgattt

SEQ ID NO: 389 - DNA, 19 bp, Artificial
  Target Sequence
gtgagtgactactccatga

SEQ ID NO: 390 - DNA, 19 bp, Artificial
  Target Sequence
gctacgtctgctactacaa

SEQ ID NO: 391 - DNA, 19 bp, Artificial
  Target Sequence
tgttcgtgagagactttga

SEQ ID NO: 392 - DNA, 19 bp, Artificial
  Target Sequence
gactttgagcagccattca

SEQ ID NO: 393 - DNA, 19 bp, Artificial
  Target Sequence
ttgagcagccattcatcaa

SEQ ID NO: 394 - DNA, 19 bp, Artificial
  Target Sequence
agcagccattcatcaacaa

SEQ ID NO: 395 - DNA, 19 bp, Artificial
  Target Sequence
tcacaggcaacgagctcta

SEQ ID NO: 396 - DNA, 19 bp, Artificial
  Target Sequence
caggcaacgagctctatga

SEQ ID NO: 397 - DNA, 19 bp, Artificial
  Target Sequence
atgtgtgcaaggccaacaa

SEQ ID NO: 398 - DNA, 19 bp, Artificial
  Target Sequence
caggagacgagctggtgaa

SEQ ID NO: 399 - DNA, 19 bp, Artificial
  Target Sequence
ccgagttccagtggtacaa

SEQ ID NO: 400 - DNA, 19 bp, Artificial
  Target Sequence
agcatctactcgcgtcaca

SEQ ID NO: 401 - DNA, 19 bp, Artificial
  Target Sequence
agcaagacctcatgccaca

SEQ ID NO: 402 - DNA, 19 bp, Artificial
  Target Sequence
gcttcaccatcgaatccaa

SEQ ID NO: 403 - DNA, 19 bp, Artificial
  Target Sequence
gccatccgaggagctacta

SEQ ID NO: 404 - DNA, 19 bp, Artificial
  Target Sequence
tgccaagccgacagctaca

SEQ ID NO: 405 - DNA, 19 bp, Artificial
  Target Sequence
cgcttctgctcgactgcaa

SEQ ID NO: 406 - DNA, 19 bp, Artificial
  Target Sequence
acaagcactgccacaagaa

SEQ ID NO: 407 - DNA, 19 bp, Artificial
  Target Sequence
tcgacttggcggactccaa

SEQ ID NO: 408 - DNA, 19 bp, Artificial
  Target Sequence
tggagatcgtgatccttgt

SEQ ID NO: 409 - DNA, 19 bp, Artificial
  Target Sequence
ccgctttcggcatccacaa

SEQ ID NO: 410 - DNA, 19 bp, Artificial
  Target Sequence
cgctgatgtcggagctcaa

SEQ ID NO: 411 - DNA, 19 bp, Artificial
  Target Sequence
ctgatgtcggagctcaaga

SEQ ID NO: 412 - DNA, 19 bp, Artificial
  Target Sequence
aaagcgacgtggtgaagat

SEQ ID NO: 413 - DNA, 19 bp, Artificial
  Target Sequence
ctgaaagcatcttcgacaa

SEQ ID NO: 414 - DNA, 19 bp, Artificial
  Target Sequence
tacgccacatcatgctgaa

SEQ ID NO: 415 - DNA, 19 bp, Artificial
  Target Sequence
gatagagagcaggcataga

SEQ ID NO: 416 - DNA, 19 bp, Artificial
  Target Sequence
gagcaggcatagacaagaa

SEQ ID NO: 417 - DNA, 19 bp, Artificial
  Target Sequence
tggttgaactctggtggca

SEQ ID NO: 418 - DNA, 19 bp, Artificial
  Target Sequence
gttgaactctggtggcaca

SEQ ID NO: 419 - DNA, 19 bp, Artificial
  Target Sequence
atgtcatttagttcagcat

SEQ ID NO: 420 - DNA, 19 bp, Artificial
  Target Sequence
ctttggcgacctcctttca

SEQ ID NO: 421 - DNA, 19 bp, Artificial
  Target Sequence
tttggcgacctcctttcat

SEQ ID NO: 422 - DNA, 19 bp, Artificial
  Target Sequence
ttggcgacctcctttcatc

SEQ ID NO: 423 - DNA, 19 bp, Artificial
  Target Sequence
tggcgacctcctttcatca

SEQ ID NO: 424 - DNA, 19 bp, Artificial
  Target Sequence
tgttggaggttaaggcata

SEQ ID NO: 425 - DNA, 19 bp, Artificial
  Target Sequence
tggaggttaaggcatacga

SEQ ID NO: 426 - DNA, 19 bp, Artificial
  Target Sequence
gttaaggcatacgagagca

SEQ ID NO: 427 - DNA, 19 bp, Artificial
  Target Sequence
ctgaccaaacagccaacta

SEQ ID NO: 428 - DNA, 19 bp, Artificial
  Target Sequence
tgaccaaacagccaactag

SEQ ID NO: 429 - DNA, 19 bp, Artificial
  Target Sequence
attatacgctggcaacaca

SEQ ID NO: 430 - DNA, 19 bp, Artificial
  Target Sequence
tatacgctggcaacacaga

SEQ ID NO: 431 - DNA, 19 bp, Artificial
  Target Sequence
gctatttcttctactgcta

SEQ ID NO: 432 - DNA, 19 bp, Artificial
  Target Sequence
cttctactgctatctacta

SEQ ID NO: 433 - DNA, 19 bp, Artificial
  Target Sequence
cttatgccagcgtgacaga

SEQ ID NO: 434 - DNA, 19 bp, Artificial
  Target Sequence
gctcacctcttgccttcta

SEQ ID NO: 435 - DNA, 19 bp, Artificial
  Target Sequence
ctaggtcacttctcacaat

SEQ ID NO: 436 - DNA, 19 bp, Artificial
  Target Sequence
cgccgattattccttggta

SEQ ID NO: 437 - DNA, 19 bp, Artificial
  Target Sequence
gccgattattccttggtaa

SEQ ID NO: 438 - DNA, 19 bp, Artificial
  Target Sequence
ccgattattccttggtaat

SEQ ID NO: 439 - DNA, 19 bp, Artificial
  Target Sequence
tccttggtaatatgagtaa

SEQ ID NO: 440 - RNA, 19 nt, Artificial
  Synthetic oligonucleotide
ggagacacguggaggauuu

SEQ ID NO: 441 - RNA, 19 nt, Artificial
  Synthetic oligonucleotide
gaugaaaccuaucagucua

SEQ ID NO: 442 - RNA, 19 nt, Artificial
  Synthetic oligonucleotide
gauaucaaaugguacagaa

SEQ ID NO: 443 - RNA, 19 nt, Artificial
  Synthetic oligonucleotide
cgacauagccuccacuguu

SEQ ID NO: 444 - DNA, 19 bp, Artificial
  Synthetic oligonucleotide
ccaaatgacttcaactata